SEQUENCE LISTING (104 sequences)

SEQ ID NO: 1 - protein, 303 aa, Ophiostoma novo-ulmi subsp. americana (mitochondrion)
MAYMSRRESINPWILTGFADAEGSFLLRIRNNNKSSVGYSTELGFQITLHNKDKSILENIQSTWKVGVIANSGDNAVSLKVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 2 - protein, 303 aa, Ophiostoma novo-ulmi subsp. americana (mitochondrion)
MAYMSRRESINPWILTGFADAEGSFLLRIRNNNKSSVGYSTELGFQITLHNKDKSILENIQSTWKVGVIANSGDNAVSLKVTRFEDLKVIIDHFEKYPLITQKLGDYKLFKQAFSVMENKEHLKENGIKELVRIKAKLNWGLTDELKKAFPENISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 3 - protein, 303 aa, Ophiostoma novo-ulmi subsp. americana (mitochondrion)
  MOD_RES (1)..(3): Any amino acid or absent
XXXMSRRESINPWILTGFADAEGSFLLRIRNNNKSSVGYSTELGFQITLHNKDKSILENIQSTWKVGVIANSGDNAVSLKVTRFEDLKVIIDHFEKYPLITQKLGDYKLFKQAFSVMENKEHLKENGIKELVRIKAKLNWGLTDELKKAFPENISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 4 - protein, 303 aa, Ophiostoma novo-ulmi subsp. americana (mitochondrion)
  MOD_RES (1)..(4): Any amino acid or absent
  MOD_RES (302)..(303): Any amino acid or absent
XXXXSRRESINPWILTGFADAEGSFLLRIRNNNKSSVGYSTELGFQITLHNKDKSILENIQSTWKVGVIANSGDNAVSLKVTRFEDLKVIIDHFEKYPLITQKLGDYKLFKQAFSVMENKEHLKENGIKELVRIKAKLNWGLTDELKKAFPENISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRXX

SEQ ID NO: 5 - protein, 303 aa, Ophiostoma novo-ulmi subsp. americana (mitochondrion)
  MOD_RES (1)..(8): Any amino acid or absent
  MOD_RES (302)..(303): Any amino acid or absent
XXXXXXXXSINPWILTGFADAEGSFLLRIRNNNKSSVGYSTELGFQITLHNKDKSILENIQSTWKVGVIANSGDNAVSLKVTRFEDLKVIIDHFEKYPLITQKLGDYKLFKQAFSVMENKEHLKENGIKELVRIKAKLNWGLTDELKKAFPENISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRXX

SEQ ID NO: 6 - protein, 303 aa, Artificial Sequence
  Synthesized I-OnuI LHE variant
  MOD_RES (1)..(4): Any amino acid or absent
  MOD_RES (302)..(303): Any amino acid or absent
XXXXSRRESINPWILTGFADAEGSFVLSIQNRNDYATGYRIHLTFQITLHNKDKSILENIQSTWKVGKINNAGDNLVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGEGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGRXX

SEQ ID NO: 7 - protein, 303 aa, Artificial Sequence
  Synthesized I-OnuI LHE variant
  MOD_RES (1)..(4): Any amino acid or absent
  MOD_RES (302)..(303): Any amino acid or absent
XXXXSRRESINPWILTGFADAEGSFVLSIQNRNDYATGYRIHLTFQITLHNKDKSILENIQSTWKVGKINNTGDNLVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGRXX

SEQ ID NO: 8 - protein, 306 aa, Artificial Sequence
  Synthesized I-OnuI LHE variant
  MOD_RES (1)..(4): Any amino acid or absent
XXXXSRRESINPWILTGFADAEGSFVLRIQNSNDYATGYRIHLTFQITLHNKDKSILENIQSTWKVGKINNTGDNLVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGRVFSGR

SEQ ID NO: 9 - protein, 308 aa, Artificial Sequence
  Synthesized I-OnuI LHE variant
  MOD_RES (1)..(4): Any amino acid or absent
  MOD_RES (307)..(308): Any amino acid or absent
XXXXSRRESINPWILTGFADAEGSFVLSIQNKNNYATGYRIHLTFQIILHNKDKSILENIQSTWKVGKINNTGDNLVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGRVFSGRXX

SEQ ID NO: 10 - protein, 308 aa, Artificial Sequence
  Synthesized I-OnuI LHE variant
  MOD_RES (1)..(4): Any amino acid or absent
  MOD_RES (307)..(308): Any amino acid or absent
XXXXSRRESINPWILTGFADAEGSFVLSIQNRNDYATGYRIRLTFQIILHNKDKSILENIQSTWKVGKINNTGDNLVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGRVFSGRXX

SEQ ID NO: 11 - protein, 308 aa, Artificial Sequence
  Synthesized I-OnuI LHE variant
  MOD_RES (1)..(4): Any amino acid or absent
  MOD_RES (307)..(308): Any amino acid or absent
XXXXSRRESINPWILTGFADAEGSFVLGIQNRNDYATGYRIRLTFQITLRNKDKSILENIQSTWKVGKINNTGDNLVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGRVFSGRXX

SEQ ID NO: 12 - protein, 308 aa, Artificial Sequence
  Synthesized I-OnuI LHE variant
  MOD_RES (1)..(4): Any amino acid or absent
  MOD_RES (307)..(308): Any amino acid or absent
XXXXSRRESINPWILTGFADAEGSFVLSIHNRNDYATGYRIHLTFQITLHNKDKSILENIQSTWKVGKINNTGDNHVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGRVFSGRXX

SEQ ID NO: 13 - protein, 308 aa, Artificial Sequence
  Synthesized I-OnuI LHE variant
  MOD_RES (1)..(4): Any amino acid or absent
  MOD_RES (307)..(308): Any amino acid or absent
XXXXSRRESINPWILTGFADAEGSFRLSIQNRNDYATGYRIHLRFQITLHNKDKSILENIQSTWKVGKINNTGDNLVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGRVFSGRXX

SEQ ID NO: 14 - protein, 308 aa, Artificial Sequence
  Synthesized I-OnuI LHE variant
  MOD_RES (1)..(4): Any amino acid or absent
  MOD_RES (307)..(308): Any amino acid or absent
XXXXSRRESINPWILTGFADAEGSFYLSIQNRNDYATGYRIHLRFQITLHNKDKSILENIQSTWKVGRIENTGDNLVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGRVFSGRXX

SEQ ID NO: 15 - protein, 308 aa, Artificial Sequence
  Synthesized I-OnuI LHE variant
  MOD_RES (1)..(4): Any amino acid or absent
  MOD_RES (307)..(308): Any amino acid or absent
XXXXSRRESINPWILTGFADAEGSFVLSIQNRSDYATGYRIHLRFQITLHNKEKSILENIQSTWKVGKINNTGDNLVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGRVFSGRXX

SEQ ID NO: 16 - protein, 308 aa, Artificial Sequence
  Synthesized I-OnuI LHE variant
  MOD_RES (1)..(4): Any amino acid or absent
  MOD_RES (307)..(308): Any amino acid or absent
XXXXSRRESINPWILTGFADAEGSFVLSIQNRNDYATGYRIHLRFQIGLHNKDKSILENIQSTWKVGKIANVGDNRVQLVVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGRVFSGRXX

SEQ ID NO: 17 - protein, 308 aa, Artificial Sequence
  Synthesized I-OnuI LHE variant
  MOD_RES (1)..(4): Any amino acid or absent
  MOD_RES (307)..(308): Any amino acid or absent
XXXXSRRESINPWILTGFADAEGSFVLSIQNRNDYATGYRIHLRFQIGLHNKDKSILENIQSTWKVGKIQNMGDNRVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGRVFSGRXX

SEQ ID NO: 18 - protein, 308 aa, Artificial Sequence
  Synthesized I-OnuI LHE variant
  MOD_RES (1)..(4): Any amino acid or absent
  MOD_RES (307)..(308): Any amino acid or absent
XXXXSRRESINPWILTGFADAEGSFVLSIQNRNDYATGYRIHLRFQIGLHNKDKSILENIQSTWKVGKILNVGDNHVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGRVFSGRXX

SEQ ID NO: 19 - protein, 308 aa, Artificial Sequence
  Synthesized I-OnuI LHE variant
  MOD_RES (1)..(4): Any amino acid or absent
  MOD_RES (307)..(308): Any amino acid or absent
XXXXSRRESINPWILTGFADAEGSFVLSIQNRNDYATGYRIHLRFQIVLHNKDKSILENIQSTWKVGKISNVGDNHVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGRVFSGRXX

SEQ ID NO: 20 - protein, 875 aa, Artificial Sequence
  Synthesized megaTAL amino acid sequence
MGSAPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGSFVLSIQNRNDYATGYRIHLTFQITLHNKDKSILENIQSTWKVGKINNAGDNLVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGEGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGR

SEQ ID NO: 21 - protein, 875 aa, Artificial Sequence
  Synthesized megaTAL amino acid sequence
MGSAPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGSFVLSIQNRNDYATGYRIHLTFQITLHNKDKSILENIQSTWKVGKINNTGDNLVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGR

SEQ ID NO: 22 - protein, 1116 aa, Artificial Sequence
  Synthesized megaTAL amino acid sequence
MGSAPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGSFVLSIQNRNDYATGYRIHLTFQITLHNKDKSILENIQSTWKVGKINNAGDNLVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGEGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGRVFASTGSEPPRAETFVFLDLEATGLPNMDPEIAEISLFAVHRSSLENPERDDSGSLVLPRVLDKLTLCMCPERPFTAKASEITGLSSESLMHCGKAGFNGAVVRTLQGFLSRQEGPICLVAHNGFDYDFPLLCTGLQRLGAHLPQDTVCLDTLPALRGLDRAHSHGTRAQGRKSYSLASLFHRYFQAEPSAAHSAEGDVHTLLLIFLHRAPELLAWADEQARSWAHIEPMYVPPDGPSLEA

SEQ ID NO: 23 - protein, 1116 aa, Artificial Sequence
  Synthesized megaTAL amino acid sequence
MGSAPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGSFVLSIQNRNDYATGYRIHLTFQITLHNKDKSILENIQSTWKVGKINNTGDNLVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGRVFASTGSEPPRAETFVFLDLEATGLPNMDPEIAEISLFAVHRSSLENPERDDSGSLVLPRVLDKLTLCMCPERPFTAKASEITGLSSESLMHCGKAGFNGAVVRTLQGFLSRQEGPICLVAHNGFDYDFPLLCTGLQRLGAHLPQDTVCLDTLPALRGLDRAHSHGTRAQGRKSYSLASLFHRYFQAEPSAAHSAEGDVHTLLLIFLHRAPELLAWADEQARSWAHIEPMYVPPDGPSLEA

SEQ ID NO: 24 - DNA, 29 bp, Homo sapiens
cctggagcctgtgataaaagcaactgtta

SEQ ID NO: 25 - DNA, 22 bp, Homo sapiens
cagttgcttttatcacaggctc

SEQ ID NO: 26 - DNA, 11 bp, Homo sapiens
agtctagtgca

SEQ ID NO: 27 - DNA, 39 bp, Homo sapiens
agtctagtgcaagcttacagttgcttttatcacaggctc

SEQ ID NO: 28 - DNA, 22 bp, Artificial Sequence
  Synthesized I-OnuI LHE variant target site
cagttgcttttataacctttta

SEQ ID NO: 29 - DNA, 22 bp, Artificial Sequence
  Synthesized I-OnuI LHE variant CTD target site
tttccacttttatcacaggctc

SEQ ID NO: 30 - DNA, 22 bp, Artificial Sequence
  Synthesized I-SmaMI target site
tatcctccattatcaggtgtac

SEQ ID NO: 31 - DNA, 22 bp, Homo sapiens
cttccaggaattctttggcctg

SEQ ID NO: 32 - DNA, 7078 bp, Artificial Sequence
  Synthesized I-OnuI LHE variant surface display plasmid
gacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtacccgacaggttatcagcaacaacacagtcatatccattctcaattagctctaccacagtgtgtgaaccaatgtatccagcaccacctgtaaccaaaacaattttagaagtactttcactttgtaactgagctgtcatttatattgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagatcgaatcctacttcatacattttcaattaagatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggctagtggtggaggaggctctggtggaggcggtagcggaggcggagggtcggctagctccatcaacccatggattctgactggtttcactgatgccgaaggatcattcatgctaagaatccgtaacacgaacaaccggtcagtagggtactacacttcactggtattcgaaatcactctgcacaacaaggacaaatcgattcttgagaatatccagtcgacttggaaggtcggcacaatcaacaaccgaggcgacggcaccgccagactgagcgtcactcgtttcgaagatttgaaagtgattatcgaccacttcgagaaatatccgctgattacccagaaattgggcgattacaagttgtttaaacaggcattcagcgtcatggagaacaaagaacatcttaaggagaatgggattaaggagctcgtacgaatcaaagctaagatgaattggggtctcaatgacgaattgaaaaaagcatttccagagaacatcagcaaagagcgcccccttatcaataagaacattccgaatctcaaatggctggctggattcacatctggtgaaggcacattctacgtgcacctagcaaagtctgaagctagcggcaaggtatacgtgcgactgaggttcataatcggccagcacatcagagacaagaacctgatgaattcattgataacatacctaggctgtggtacgatccaggagaagaacaggtctaagggcagtatgctccacttcatagtaactaaattcagcgatatcaacgacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgttctagagaacaaaagttaatttctgaagaggacttgtaagatctgataacaacagtgtagatgtaacaaaatcgactttgttcccactgtacttttagctcgtacaaaatacaatatacttttcatttctccgtaaacaacatgttttcccatgtaatatccttttctatttttcgttccgttaccaactttacacatactttatatagctattcacttctatacactaaaaaactaagacaattttaattttgctgcctgccatatttcaatttgttataaattcctataatttatcctattagtagctaaaaaaagatgaatgtgaatcgaatcctaagagaattgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcctttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcaggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatcaagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgtctattaaatgcttcctatattatatatatagtaatgtcgtttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 33 - DNA, 22 bp, Artificial Sequence
  Synthesized polynucleotide sequence for a central 4 array for an I-OnuI LHE variant
  misc_feature (10)..(13): n is a, c, g, or t
cttccaggannnntttggcctg

SEQ ID NO: 34 - DNA, 7243 bp, Artificial Sequence
  Synthesized polynucleotide sequence of an I-OnuI LHE variant surface display plasmid
gacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtacccgacaggttatcagcaacaacacagtcatatccattctcaattagctctaccacagtgtgtgaaccaatgtatccagcaccacctgtaaccaaaacaattttagaagtactttcactttgtaactgagctgtcatttatattgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagatcgaatcctacttcatacattttcaattaagatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggctagtggtggaggaggctctggtggaggcggtagcggaggcggagggtcggctagctccatcaacccatggattctgactggtttcgctgatgccgaaggaagcttcgtgctaagtatccaaaacagaaacgattatgctactggttacagaattcacctgacattccaaatcaccctgcacaacaaggacaaatcgattctggagaatatccagtcgacttggaaggtcggcaaaatcaataacacgggcgacaacctcgtccaactgagagtctaccgtttcgaagatttgaaagtgattatcgaccacttcgagaaatatccgctgataacacagaaattgggcgattacaagttgtttaaacaggcattcagcgtcatggagaacaaagaacatcttaaggagaatgggattaaggagctcgtacgaatcaaagctaagatgaattggggtctcaatgacgaattgaaaaaagcatttccagagaacattagcaaagagcgcccccttatcaataagaacattccgaatttcaaatggctggctggattcacatctggtgatggctccttcttcgtgcgcctaagaaagtctaatgttaatgctagagtacgtgtgcaactggtattcgagatctcacagcacatcagagacaagaacctgatgaattcattgataacatacctaggctgtggtcacatctacgagggaaacaaatctgagcgcagttggctccaattcagagtagaaaaattcagcgatatcaacgacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgtgtcttctctagaggcggttccagaagcggatctggtactggcgaacagaaactcataagcgaagaagaccttagcgggactggagagcaaaagttgatttctgaggaggatttgtcgggaaccggggagcagaagttaatcagtgaagaggatctcagtggaacgggcgaacaaaagttgatctcggaggaagacttataatgagatctgataacaacagtgtagatgtaacaaaatcgactttgttcccactgtacttttagctcgtacaaaatacaatatacttttcatttctccgtaaacaacatgttttcccatgtaatatccttttctatttttcgttccgttaccaactttacacatactttatatagctattcacttctatacactaaaaaactaagacaattttaattttgctgcctgccatatttcaatttgttataaattcctataatttatcctattagtagctaaaaaaagatgaatgtgaatcgaatcctaagagaattgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcctttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcaggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgacatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatcaagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgtctattaaatgcttcctatattatatatatagtaatgtcgtttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 35 - DNA, 22 bp, Artificial Sequence
  Synthesized polynucleotide sequence for a central 4 array for an I-OnuI LHE variant
  misc_feature (10)..(13): n is a, c, g, or t
cagttgcttnnnncacaggctc

SEQ ID NO: 36 - RNA, 2634 nt, Artificial Sequence
  Synthesized mRNA encoding a megaTAL
augggauccgcgccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaacuuaacacccgaucaagugguagcgauagcgucaaauaucggggguaaacaggcuuuggagacgguacagcgguuauugccgguacucugccaggaccacggauugacaccggaccaagugguggcgauugcguccaauaacggaggcaagcaggcacuagagaccguccaacggcuucuucccguucuuugucaggaucaugggcuaaccccugaucagguagucgcuauagcuucaaauggcgggggcaagcaagcacuggagaccguucaacgacuccugccagugcucugccaagaccacggacuuacgccagaucaggugguugcuauugccucccacgauggcgggaaacaagcguuggaaacugugcagagacuguuaccugucuugugucaagaccacggccucacgccagaucaggugguagccauagcgucgaauggaggugguaagcaagcccuugaaacgguccagcgucuucugccgguguugugccaggaccacggacuaacgccggaucaggucguagccauugcuucaaauaucggcggcaaacaggcgcuagagacaguccagcgccucuugccuguguuaugccaggaucacggcuuaaccccagaccaaguuguggcuauugcaucuaacaaugguggcaaacaagccuuggagacagugcaacgauuacugccugucuuaugucaggaucauggccugacgcccgaucagguaguggcaaucgcaucuaacggcggagguaagcaagcacuggagacuguccagagauuguuacccguacuaugucaagaucaugguuugacgccugaucagguuguugcgauagccagcaacaacggagggaaacaggcucuugaaaccguacagcgacuucucccagucuugugccaagaucacgggcuuacuccugaucaagucguagcuaucgccagccacgacggugggaaacaggcccuggaaaccguacaacgucuccucccaguacuuugucaagaccacggguugacuccggaucaagucgucgcgaucgcgagcaauauaggggggaagcaggcgcuggaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucucgcagagaguccaucaacccauggauucugacugguuucgcugaugccgaaggaagcuucgugcuaaguauccaaaacagaaacgauuaugcuacugguuacagaauucaccugacauuccaaaucacccugcacaacaaggacaaaucgauucuggagaauauccagucgacuuggaaggucggcaaaaucaauaacacgggcgacaaccucguccaacugagagucuaccguuucgaagauuugaaagugauuaucgaccacuucgagaaauauccgcugauaacacagaaauugggcgauuacaaguuguuuaaacaggcauucagcgucauggagaacaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauuccgaauuucaaauggcuggcuggauucacaucuggugauggcuccuucuucgugcgccuaagaaagucuaauguuaaugcuagaguacgugugcaacugguauucgagaucucacagcacaucagagacaagaaccugaugaauucauugauaacauaccuaggcuguggucacaucuacgagggaaacaaaucugagcgcaguuggcuccaauucagaguagaaaaauucagcgauaucaacgacaagaucauuccgguauuccaggaaaauacucugauuggcgucaaacucgaggacuuugaagauuggugcaagguugccaaauugaucgaagagaagaaacaccugaccgaauccgguuuggaugagauuaagaaaaucaagcugaacaugaacaaaggucgugucuucuaa

SEQ ID NO: 37 - RNA, 3351 nt, Artificial Sequence
  Synthesized mRNA encoding a megaTAL
augggauccgcgccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaacuuaacacccgaucaagugguagcgauagcgucaaauaucggggguaaacaggcuuuggagacgguacagcgguuauugccgguacucugccaggaccacggauugacaccggaccaagugguggcgauugcguccaauaacggaggcaagcaggcacuagagaccguccaacggcuucuucccguucuuugucaggaucaugggcuaaccccugaucagguagucgcuauagcuucaaauggcgggggcaagcaagcacuggagaccguucaacgacuccugccagugcucugccaagaccacggacuuacgccagaucaggugguugcuauugccucccacgauggcgggaaacaagcguuggaaacugugcagagacuguuaccugucuugugucaagaccacggccucacgccagaucaggugguagccauagcgucgaauggaggugguaagcaagcccuugaaacgguccagcgucuucugccgguguugugccaggaccacggacuaacgccggaucaggucguagccauugcuucaaauaucggcggcaaacaggcgcuagagacaguccagcgccucuugccuguguuaugccaggaucacggcuuaaccccagaccaaguuguggcuauugcaucuaacaaugguggcaaacaagccuuggagacagugcaacgauuacugccugucuuaugucaggaucauggccugacgcccgaucagguaguggcaaucgcaucuaacggcggagguaagcaagcacuggagacuguccagagauuguuacccguacuaugucaagaucaugguuugacgccugaucagguuguugcgauagccagcaacaacggagggaaacaggcucuugaaaccguacagcgacuucucccagucuugugccaagaucacgggcuuacuccugaucaagucguagcuaucgccagccacgacggugggaaacaggcccuggaaaccguacaacgucuccucccaguacuuugucaagaccacggguugacuccggaucaagucgucgcgaucgcgagcaauauaggggggaagcaggcgcuggaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucucgcagagaguccaucaacccauggauucugacugguuucgcugaugccgaaggaagcuucgugcuaaguauccaaaacagaaacgauuaugcuacugguuacagaauucaccugacauuccaaaucacccugcacaacaaggacaaaucgauucuggagaauauccagucgacuuggaaggucggcaaaaucaauaacacgggcgacaaccucguccaacugagagucuaccguuucgaagauuugaaagugauuaucgaccacuucgagaaauauccgcugauaacacagaaauugggcgauuacaaguuguuuaaacaggcauucagcgucauggagaacaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauuccgaauuucaaauggcuggcuggauucacaucuggugauggcuccuucuucgugcgccuaagaaagucuaauguuaaugcuagaguacgugugcaacugguauucgagaucucacagcacaucagagacaagaaccugaugaauucauugauaacauaccuaggcuguggucacaucuacgagggaaacaaaucugagcgcaguuggcuccaauucagaguagaaaaauucagcgauaucaacgacaagaucauuccgguauuccaggaaaauacucugauuggcgucaaacucgaggacuuugaagauuggugcaagguugccaaauugaucgaagagaagaaacaccugaccgaauccgguuuggaugagauuaagaaaaucaagcugaacaugaacaaaggucgugucuucgcuagcaccgguucugagccaccucgggcugagaccuuuguauuccuggaccuagaagccacugggcucccaaacauggacccugagauugcagagauaucccuuuuugcuguucaccgcucuucccuggagaacccagaacgggaugauucugguuccuuggugcugccccguguucuggacaagcucacacugugcaugugcccggagcgccccuuuacugccaaggccagugagauuacugguuugagcagcgaaagccugaugcacugcgggaaggcugguuucaauggcgcugugguaaggacacugcagggcuuccuaagccgccaggagggccccaucugccuuguggcccacaauggcuucgauuaugacuucccacugcugugcacggggcuacaacgucugggugcccaucugccccaagacacugucugccuggacacacugccugcauugcggggccuggaccgugcucacagccacggcaccagggcucaaggccgcaaaagcuacagccuggccagucucuuccaccgcuacuuccaggcugaacccagugcugcccauucagcagaaggugaugugcacacccugcuucugaucuuccugcaucgugcuccugagcugcucgccugggcagaugagcaggcccgcagcugggcucauauugagcccauguacgugccaccugaugguccaagccucgaagccuga

SEQ ID NO: 38 - RNA, 711 nt, Mus musculus
augucugagccaccucgggcugagaccuuuguauuccuggaccuagaagccacugggcucccaaacauggacccugagauugcagagauaucccuuuuugcuguucaccgcucuucccuggagaacccagaacgggaugauucugguuccuuggugcugccccguguucuggacaagcucacacugugcaugugcccggagcgccccuuuacugccaaggccagugagauuacugguuugagcagcgaaagccugaugcacugcgggaaggcugguuucaauggcgcugugguaaggacacugcagggcuuccuaagccgccaggagggccccaucugccuuguggcccacaauggcuucgauuaugacuucccacugcugugcacggggcuacaacgucugggugcccaucugccccaagacacugucugccuggacacacugccugcauugcggggccuggaccgugcucacagccacggcaccagggcucaaggccgcaaaagcuacagccuggccagucucuuccaccgcuacuuccaggcugaacccagugcugcccauucagcagaaggugaugugcacacccugcuucugaucuuccugcaucgugcuccugagcugcucgccugggcagaugagcaggcccgcagcugggcucauauugagcccauguacgugccaccugaugguccaagccucgaagccuga

SEQ ID NO: 39 - protein, 236 aa, Mus musculus
MSEPPRAETFVFLDLEATGLPNMDPEIAEISLFAVHRSSLENPERDDSGSLVLPRVLDKLTLCMCPERPFTAKASEITGLSSESLMHCGKAGFNGAVVRTLQGFLSRQEGPICLVAHNGFDYDFPLLCTGLQRLGAHLPQDTVCLDTLPALRGLDRAHSHGTRAQGRKSYSLASLFHRYFQAEPSAAHSAEGDVHTLLLIFLHRAPELLAWADEQARSWAHIEPMYVPPDGPSLEA

SEQ ID NO: 40 - protein, 3 aa, Artificial Sequence
  Exemplary linker sequence
GGG

SEQ ID NO: 41 - protein, 5 aa, Artificial Sequence
  Exemplary linker sequence
DGGGS

SEQ ID NO: 42 - protein, 5 aa, Artificial Sequence
  Exemplary linker sequence
TGEKP

SEQ ID NO: 43 - protein, 4 aa, Artificial Sequence
  Exemplary linker sequence
GGRR

SEQ ID NO: 44 - protein, 5 aa, Artificial Sequence
  Exemplary linker sequence
GGGGS

SEQ ID NO: 45 - protein, 14 aa, Artificial Sequence
  Exemplary linker sequence
EGKSSGSGSESKVD

SEQ ID NO: 46 - protein, 18 aa, Artificial Sequence
  Exemplary linker sequence
KESGSVSSEQLAQFRSLD

SEQ ID NO: 47 - protein, 8 aa, Artificial Sequence
  Exemplary linker sequence
GGRRGGGS

SEQ ID NO: 48 - protein, 9 aa, Artificial Sequence
  Exemplary linker sequence
LRQRDGERP

SEQ ID NO: 49 - protein, 12 aa, Artificial Sequence
  Exemplary linker sequence
LRQKDGGGSERP

SEQ ID NO: 50 - protein, 16 aa, Artificial Sequence
  Exemplary linker sequence
LRQKDGGGSGGGSERP

SEQ ID NO: 51 - protein, 7 aa, Artificial Sequence
  Cleavage sequence by TEV protease
  misc_feature (2)..(3): Xaa is any amino acid
  misc_feature (5)..(5): Xaa is any amino acid
  MISC_FEATURE (7)..(7): Xaa = Gly or Ser
EXXYXQX

SEQ ID NO: 52 - protein, 7 aa, Artificial Sequence
  Cleavage sequence by TEV protease
ENLYFQG

SEQ ID NO: 53 - protein, 7 aa, Artificial Sequence
  Cleavage sequence by TEV protease
ENLYFQS

SEQ ID NO: 54 - protein, 22 aa, Artificial Sequence
  Self-cleaving polypeptide comprising 2A site
GSGATNFSLLKQAGDVEENPGP

SEQ ID NO: 55 - protein, 19 aa, Artificial Sequence
  Self-cleaving polypeptide comprising 2A site
ATNFSLLKQAGDVEENPGP

SEQ ID NO: 56 - protein, 14 aa, Artificial Sequence
  Self-cleaving polypeptide comprising 2A site
LLKQAGDVEENPGP

SEQ ID NO: 57 - protein, 21 aa, Artificial Sequence
  Self-cleaving polypeptide comprising 2A site
GSGEGRGSLLTCGDVEENPGP

SEQ ID NO: 58 - protein, 18 aa, Artificial Sequence
  Self-cleaving polypeptide comprising 2A site
EGRGSLLTCGDVEENPGP

SEQ ID NO: 59 - protein, 13 aa, Artificial Sequence
  Self-cleaving polypeptide comprising 2A site
LLTCGDVEENPGP

SEQ ID NO: 60 - protein, 23 aa, Artificial Sequence
  Self-cleaving polypeptide comprising 2A site
GSGQCTNYALLKLAGDVESNPGP

SEQ ID NO: 61 - protein, 20 aa, Artificial Sequence
  Self-cleaving polypeptide comprising 2A site
QCTNYALLKLAGDVESNPGP

SEQ ID NO: 62 - protein, 14 aa, Artificial Sequence
  Self-cleaving polypeptide comprising 2A site
LLKLAGDVESNPGP

SEQ ID NO: 63 - protein, 25 aa, Artificial Sequence
  Self-cleaving polypeptide comprising 2A site
GSGVKQTLNFDLLKLAGDVESNPGP

SEQ ID NO: 64 - protein, 22 aa, Artificial Sequence
  Self-cleaving polypeptide comprising 2A site
VKQTLNFDLLKLAGDVESNPGP

SEQ ID NO: 65 - protein, 14 aa, Artificial Sequence
  Self-cleaving polypeptide comprising 2A site
LLKLAGDVESNPGP

SEQ ID NO: 66 - protein, 19 aa, Artificial Sequence
  Self-cleaving polypeptide comprising 2A site
LLNFDLLKLAGDVESNPGP

SEQ ID NO: 67 - protein, 19 aa, Artificial Sequence
  Self-cleaving polypeptide comprising 2A site
TLNFDLLKLAGDVESNPGP

SEQ ID NO: 68 - protein, 14 aa, Artificial Sequence
  Self-cleaving polypeptide comprising 2A site
LLKLAGDVESNPGP

SEQ ID NO: 69 - protein, 17 aa, Artificial Sequence
  Self-cleaving polypeptide comprising 2A site
NFDLLKLAGDVESNPGP

SEQ ID NO: 70 - protein, 20 aa, Artificial Sequence
  Self-cleaving polypeptide comprising 2A site
QLLNFDLLKLAGDVESNPGP

SEQ ID NO: 71 - protein, 24 aa, Artificial Sequence
  Self-cleaving polypeptide comprising 2A site
APVKQTLNFDLLKLAGDVESNPGP

SEQ ID NO: 72 - protein, 40 aa, Artificial Sequence
  Self-cleaving polypeptide comprising 2A site
VTELLYRMKRAETYCPRPLLAIHPTEARHKQKIVAPVKQT

SEQ ID NO: 73 - protein, 18 aa, Artificial Sequence
  Self-cleaving polypeptide comprising 2A site
LNFDLLKLAGDVESNPGP

SEQ ID NO: 74 - protein, 40 aa, Artificial Sequence
  Self-cleaving polypeptide comprising 2A site
LLAIHPTEARHKQKIVAPVKQTLNFDLLKLAGDVESNPGP

SEQ ID NO: 75 - protein, 33 aa, Artificial Sequence
  Self-cleaving polypeptide comprising 2A site
EARHKQKIVAPVKQTLNFDLLKLAGDVESNPGP

SEQ ID NO: 76 - DNA, 10 bp, Artificial Sequence
  Consensus Kozak sequence
gccrccatgg

SEQ ID NO: 77 - DNA, 29 bp, Homo sapiens
taacagttgcttttatcacaggctccagg

SEQ ID NO: 78 - DNA, 29 bp, Homo sapiens
attctcaacgaaaatagtgtccgaggtcc

SEQ ID NO: 79 - protein, 293 aa, Ophiostoma novo-ulmi subsp. americana (mitochondrion)
SINPWILTGFADAEGSFLLRIRNNNKSSVGYSTELGFQITLHNKDKSILENIQSTWKVGVIANSGDNAVSLKVTRFEDLKVIIDHFEKYPLITQKLGDYKLFKQAFSVMENKEHLKENGIKELVRIKAKLNWGLTDELKKAFPENISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGR

SEQ ID NO: 80 - protein, 293 aa, Artificial Sequence
  Synthesized I-OnuI LHE variant
SINPWILTGFADAEGSFVLSIQNRNDYATGYRIHLTFQITLHNKDKSILENIQSTWKVGKINNAGDNLVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGEGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGR

SEQ ID NO: 81 - protein, 293 aa, Artificial Sequence
  Synthesized I-OnuI LHE variant
SINPWILTGFADAEGSFVLSIQNRNDYATGYRIHLTFQITLHNKDKSILENIQSTWKVGKINNTGDNLVQLRVYRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFFVRLRKSNVNARVRVQLVFEISQHIRDKNLMNSLITYLGCGHIYEGNKSERSWLQFRVEKFSDINDKIIPVFQENTLIGVKLEDFEDWCKVAKLIEEKKHLTESGLDEIKKIKLNMNKGR

SEQ ID NO: 82 - DNA, 47 bp, Homo sapiens
agctagtctagtgcaagctaacagttgcttttatcacaggctccagg

SEQ ID NO: 83 - DNA, 47 bp, Homo sapiens
tcgatcagatcacgttcgattctcaacgaaaatagtgtccgaggtcc

SEQ ID NO: 84 - DNA, 21 bp, Homo sapiens
caaaccctcctggagcctgtg

SEQ ID NO: 85 - DNA, 28 bp, Homo sapiens
caaaccctcctggagcctgtggggataa

SEQ ID NO: 86 - DNA, 21 bp, Homo sapiens
caaaccctcctggggcctgtg

SEQ ID NO: 87 - DNA, 21 bp, Homo sapiens
caaaccctcctggagcccgtg

SEQ ID NO: 88 - DNA, 18 bp, Homo sapiens
caaaccctcctggagccc

SEQ ID NO: 89 - DNA, 12 bp, Homo sapiens
caaaccctcctg

SEQ ID NO: 90 - DNA, 20 bp, Homo sapiens
caaaccctcctggagccggt

SEQ ID NO: 91 - DNA, 20 bp, Homo sapiens
caaaccctcctggagcctgg

SEQ ID NO: 92 - DNA, 19 bp, Homo sapiens
caaaccctcctggagcctg

SEQ ID NO: 93 - DNA, 22 bp, Homo sapiens
caaaccctcctggagcctgtgg

SEQ ID NO: 94 - DNA, 25 bp, Homo sapiens
aagcaactgttagcttgcactagac

SEQ ID NO: 95 - DNA, 25 bp, Homo sapiens
aagcgactgttagcttgcactagac

SEQ ID NO: 96 - DNA, 31 bp, Homo sapiens
ataaggaagcaactgttagcttgcactagac

SEQ ID NO: 97 - DNA, 26 bp, Homo sapiens
aaagcaactgttagcttgcactagac

SEQ ID NO: 98 - DNA, 16 bp, Homo sapiens
ttagcttgcactagac

SEQ ID NO: 99 - DNA, 15 bp, Homo sapiens
tagcttgcactagac

SEQ ID NO: 100 - DNA, 18 bp, Homo sapiens
tgttagcttgcactagac

SEQ ID NO: 101 - DNA, 22 bp, Homo sapiens
caactgttagcttgcactagac

SEQ ID NO: 102 - DNA, 14 bp, Homo sapiens
agcttgcactagac

SEQ ID NO: 103 - DNA, 20 bp, Homo sapiens
actgttagcttgcactagac

SEQ ID NO: 104 - DNA, 25 bp, Homo sapiens
aagcaactgttagcttgcactggac

(19) United States
(12) Patent Application Publication
(10) Pub. No.: US 2019/0184035 A1
(43) Pub. Date: Jun. 20, 2019

(54) BCL11A HOMING ENDONUCLEASE VARIANTS, COMPOSITIONS, AND METHODS OF USE

(71) Applicant: bluebird bio, Inc., Cambridge, MA (US)

(72) Inventors: Jordan JARJOUR, Seattle, WA (US); Jasdeep MANN, Seattle, WA (US)

(73) Assignee: bluebird bio, Inc., Cambridge, MA (US)

(21) Appl. No.: 16/320,280

(22) PCT Filed: Jul. 25, 2017
(86) PCT No.: PCT/US2017/043726

§ 371 Date: Jan. 24, 2019

Related U.S. Application Data
(60) Provisional application No. 62/414,273, filed on Oct. 28, 2016.
(60) Provisional application No. 62/375,829, filed on Aug. 16, 2016.
(60) Provisional application No. 62/367,465, filed on Jul. 27, 2016.
(60) Provisional application No. 62/366,530, filed on Jul. 25, 2016.

(51) Int. Cl. A61K 48/00; A61P 7/00; C12N 9/16

(52) U.S. Cl. CPC A61K 48/0066; A61P 7/00; C12Y 301/21; A61K 48/0058; C12N 9/16; A61K 48/0091

(57) ABSTRACT

The present disclosure provides improved genome editing compositions and methods for editing a BCL11A gene. The disclosure further provides genome edited cells for the prevention, treatment, or amelioration of at least one symptom of a hemoglobinopathy.

### CROSS REFERENCE TO RELATED APPLICATIONS

This application claims the benefit under 35 U.S.C. § 119(e) of U.S. Provisional Application No. 62/414,273, filed Oct. 28, 2016, U.S. Provisional Application No. 62/375,829, filed Aug. 16, 2016, U.S. Provisional Application No. 62/367,465, filed Jul. 27, 2016, U.S. Provisional Application No. 62/366,530, filed Jul. 25, 2016, each of which is incorporated by reference herein in its entirety.

### STATEMENT REGARDING SEQUENCE LISTING

The Sequence Listing associated with this application is provided in text format in lieu of a paper copy, and is hereby incorporated by reference into the specification.

The name of the text file containing the Sequence Listing is BLBD_071_04WO_ST25.txt. The text file is 141 KB, was created on Jul. 25, 2017, and is being submitted electronically via EFS-Web, concurrent with the filing of the specification.

### BACKGROUND

### Technical Field

The present disclosure relates to improved genome editing compositions. More particularly, the disclosure relates to reprogrammed nucleases, compositions, and methods of using the same for editing the B Cell CLL/Lymphoma 11A (BCL11A) gene.

### Description of the Related Art

Hemoglobinopathies are a diverse group of inherited monogenetic blood disorders that result from variations in the structure and/or synthesis of hemoglobin. The most common hemoglobinopathies are sickle cell disease (SCD), α-thalassemia, and β-thalassemia. Approximately 5% of the world's population carries a globin gene mutation. The World Health Organization estimates that more than 300,000 infants are born each year with major hemoglobin disorders. Hemoglobinopathies manifest highly variable clinical manifestations that range from mild hypochromic anemia to moderate hematological disease to severe, lifelong, transfusion-dependent anemia with multiorgan involvement.

The only potentially curative treatment available for hemoglobinopathies is allogeneic hematopoietic stem cell transplantation. However, it is estimated that HLA-compatible HSC transplants are available to less than 20% of affected individuals and long term toxicities are substantial. In addition, HSC transplants are also associated with significant mortality and morbidity in subjects that have SCD or severe thalassemias. The significant mortality and morbidity is due in part to pre-HSC transplantation transfusion-related iron overload, graft-versus-host disease (GVHD), and high doses of chemotherapy/radiation required for pre-transplant conditioning of the subject, among others.

Supportive treatments for hemoglobinopathies include periodic blood transfusions for life, combined with iron chelation, and in some cases splenectomy. Additional treatments for SCD include analgesics, antibiotics, ACE inhibitors, and hydroxyurea. However, the side effects associated with hydroxyurea treatment include cytopenia, hyperpigmentation, weight gain, opportunistic infections, azoospermia, hypomagnesemia, and cancer.

At best, patients treated with existing methods have a projected lifespan of 50 to 60 years.

### BRIEF SUMMARY

The present disclosure generally relates, in part, to compositions comprising homing endonuclease variants and megaTALs that cleave a target site in the human BCL11A gene and methods of using the same.

In various embodiments, the present disclosure contemplates, in part, a polypeptide comprising a homing endonuclease (HE) variant that cleaves a target site in the human B-cell lymphoma/leukemia 11A (BCL11A) gene.

In particular embodiments, the HE variant is an LAGLIDADG homing endonuclease (LHE) variant.

In some embodiments, the polypeptide comprises a biologically active fragment of the HE variant.

In certain embodiments, the biologically active fragment lacks the 1, 2, 3, 4, 5, 6, 7, or 8 N-terminal amino acids compared to a corresponding wild type HE.

In further embodiments, the biologically active fragment lacks the 4 N-terminal amino acids compared to a corresponding wild type HE.

In certain embodiments, the biologically active fragment lacks the 8 N-terminal amino acids compared to a corresponding wild type HE.

In additional embodiments, the biologically active fragment lacks the 1, 2, 3, 4, or 5 C-terminal amino acids compared to a corresponding wild type HE.

In certain embodiments, the biologically active fragment lacks the C-terminal amino acid compared to a corresponding wild type HE.

In particular embodiments, the biologically active fragment lacks the 2 C-terminal amino acids compared to a corresponding wild type HE.

In some embodiments, the HE variant is a variant of an LHE selected from the group consisting of: I-CreI and I-SceI.

In some embodiments, the HE variant is a variant of an LHE selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, and I-Vdil41I.

In further embodiments, the HE variant is a variant of an LHE selected from the group consisting of: I-CpaMI, I-HjeMI, I-OnuI, I-PanMI, and SmaMI.

In particular embodiments, the HE variant is an I-OnuI LHE variant.

In certain embodiments, the HE variant comprises one or more amino acid substitutions in the DNA recognition interface at amino acid positions selected from the group consisting of: 19, 24, 26, 28, 30, 32, 34, 35, 36, 37, 38, 40, 42, 44, 46, 48, 68, 70, 72, 75, 76 77, 78, 80, 82, 168, 180, 182, 184, 186, 188, 189, 190, 191, 192, 193, 195, 197, 199, 201, 203, 223, 225, 227, 229, 231, 232, 234, 236, 238, and 240 of an I-OnuI LHE amino acid sequence as set forth in SEQ ID NOs: 1-5, or a biologically active fragment thereof.

In some embodiments, the HE variant comprises at least 5, at least 15, preferably at least 25, more preferably at least 35, or even more preferably at least 40 or more amino acid substitutions in the DNA recognition interface at amino acid positions selected from the group consisting of: 19, 24, 26, 28, 30, 32, 34, 35, 36, 37, 38, 40, 42, 44, 46, 48, 68, 70, 72, 75, 76 77, 78, 80, 82, 168, 180, 182, 184, 186, 188, 189, 190, 191, 192, 193, 195, 197, 199, 201, 203, 223, 225, 227, 229, 231, 232, 234, 236, 238, and 240 of an I-OnuI LHE amino acid sequence as set forth in SEQ ID NOs: 1-5, or a biologically active fragment thereof.

In particular embodiments, the HE variant comprises at least 5, at least 15, preferably at least 25, more preferably at least 35, or even more preferably at least 40 or more amino acid substitutions at amino acid positions selected from the group consisting of: 26, 28, 30, 32, 34, 35, 36, 37, 40, 41, 42, 44, 48, 50, 53, 68, 70, 72, 76, 78, 80, 82, 138, 143, 159, 178, 180, 184, 186, 189, 190, 191, 192, 193, 195, 201, 203, 207, 223, 225, 227, 232, 236, 238, and 240 of an I-OnuI LHE amino acid sequence as set forth in SEQ ID NOs: 1-19, or a biologically active fragment thereof.

In further embodiments, the HE variant comprises at least 5, at least 15, preferably at least 25, more preferably at least 35, or even more preferably at least 40 or more of the following amino acid substitutions: L26V, L26R, L26Y, R28S, R28G, R30Q, R30H, N32R, N32S, N32K, N33S, K34D, K34N, S35Y, S36A, V37T, S40R, T41I, E42H, E42R, G44T, G44R, T48I, T48G, T48V, H50R, D53E, V68K, V68R, A70N, A70E, A70N, A70Q, A70L, A70S, S72A, S72T, S72V, S72M, A76L, A76H, A76R, S78Q, K80R, K80V, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

In certain embodiments, the HE variant comprises the following amino acid substitutions: L26V, R28S, R30Q, N32R, K34D, S35Y, S36A, V37T, S40R, T41I, E42H, G44T, V68K, A70N, S72A, A76L, S78Q, K80R, T82Y, L138M, T143N, S159P, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

In particular embodiments, the HE variant comprises the following amino acid substitutions: L26V, R28S, R30Q, N32R, K34D, S35Y, S36A, V37T, S40R, T41I, E42H, G44T, V68K, A70N, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

In some embodiments, the HE variant comprises the following amino acid substitutions: L26V, R30Q, N32S, K34D, S35Y, S36A, V37T, S40R, T41I, E42H, G44T, V68K, A70N, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

In certain embodiments, the HE variant comprises the following amino acid substitutions: L26V, R28S, R30Q, N32K, K34N, S35Y, S36A, V37T, S40R, T41I, E42H, G44T, T48I, V68K, A70N, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

In particular embodiments, the HE variant comprises the following amino acid substitutions: L26V, R28S, R30Q, N32R, K34D, S35Y, S36A, V37T, S40R, T41I, E42R, G44T, T48I, V68K, A70N, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

In additional embodiments, the HE variant comprises the following amino acid substitutions: L26V, R28G, R30Q, N32R, K34D, S35Y, S36A, V37T, S40R, T41I, E42R, G44T, H50R, V68K, A70N, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

In particular embodiments, the HE variant comprises the following amino acid substitutions: L26V, R28S, R30H, N32R, K34D, S35Y, S36A, V37T, S40R, T41I, E42H, G44R, V68K, A70N, S72T, A76H, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

In certain embodiments, the HE variant comprises the following amino acid substitutions: L26R, R28S, R30Q, N32R, K34D, S35Y, S36A, V37T, S40R, T41I, E42H, G44R, V68K, A70N, S72TA76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

In particular embodiments, the HE variant comprises the following amino acid substitutions: L26Y, R28S, R30Q, N32R, K34D, S35Y, S36A, V37T, S40R, T41I, E42H, G44R, D53E, V68R, A70E, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

In some embodiments, the HE variant comprises the following amino acid substitutions: L26V, R28S, R30Q, N32R, N33S, K34D, S35Y, S36A, V37T, S40R, T41I, E42H, G44R, D53E, V68K, A70N, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

In certain embodiments, the HE variant comprises the following amino acid substitutions: L26V, R28S, R30Q, N32R, N33S, K34D, S35Y, S36A, V37T, S40R, T41I, E42H, G44R, T48G, V68K, S72V, A76R, S78Q, K80V, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

In certain embodiments, the HE variant comprises the following amino acid substitutions: L26V, R28S, R30Q, N32R, N33S, K34D, S35Y, S36A, V37T, S40R, T41I, E42H, G44R, T48G, V68K, A70Q, S72M, A76R, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

In particular embodiments, the HE variant comprises the following amino acid substitutions: L26V, R28S, R30Q, N32R, N33S, K34D, S35Y, S36A, V37T, S40R, T41I, E42H, G44R, T48G, V68K, A70L, S72V, A76H, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

In particular embodiments, the HE variant comprises the following amino acid substitutions: L26V, R28S, R30Q, N32R, N33S, K34D, S35Y, S36A, V37T, S40R, T41I, E42H, G44R, T48V, V68K, A70S, S72V, A76H, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

In certain embodiments, the HE variant comprises an amino acid sequence that is at least 80%, preferably at least 85%, more preferably at least 90%, or even more preferably at least 95% identical to the amino acid sequence set forth in any one of SEQ ID NOs: 6-19, or a biologically active fragment thereof.

In particular embodiments, the HE variant comprises the amino acid sequence set forth in SEQ ID NO: 6, or a biologically active fragment thereof.

In some embodiments, the HE variant comprises the amino acid sequence set forth in SEQ ID NO: 7, or a biologically active fragment thereof.

In some embodiments, the HE variant comprises the amino acid sequence set forth in SEQ ID NO: 8, or a biologically active fragment thereof.

In some embodiments, the HE variant comprises the amino acid sequence set forth in SEQ ID NO: 9, or a biologically active fragment thereof.

In some embodiments, the HE variant comprises the amino acid sequence set forth in SEQ ID NO: 10, or a biologically active fragment thereof.

In some embodiments, the HE variant comprises the amino acid sequence set forth in SEQ ID NO: 11, or a biologically active fragment thereof.

In some embodiments, the HE variant comprises the amino acid sequence set forth in SEQ ID NO: 12, or a biologically active fragment thereof.

In some embodiments, the HE variant comprises the amino acid sequence set forth in SEQ ID NO: 13, or a biologically active fragment thereof.

In some embodiments, the HE variant comprises the amino acid sequence set forth in SEQ ID NO: 14, or a biologically active fragment thereof.

In some embodiments, the HE variant comprises the amino acid sequence set forth in SEQ ID NO: 15, or a biologically active fragment thereof.

In some embodiments, the HE variant comprises the amino acid sequence set forth in SEQ ID NO: 16, or a biologically active fragment thereof.

In some embodiments, the HE variant comprises the amino acid sequence set forth in SEQ ID NO: 17, or a biologically active fragment thereof.

In some embodiments, the HE variant comprises the amino acid sequence set forth in SEQ ID NO: 18, or a biologically active fragment thereof.

In some embodiments, the HE variant comprises the amino acid sequence set forth in SEQ ID NO: 19, or a biologically active fragment thereof.

In some embodiments, the polypeptide further comprises a DNA binding domain.

In further embodiments, the DNA binding domain is selected from the group consisting of: a TALE DNA binding domain and a zinc finger DNA binding domain.

In additional embodiments, the TALE DNA binding domain comprises about 9.5 TALE repeat units to about 11.5 TALE repeat units.

In additional embodiments, the TALE DNA binding domain comprises about 9.5 TALE repeat units to about 12.5 TALE repeat units.

In additional embodiments, the TALE DNA binding domain comprises about 9.5 TALE repeat units to about 13.5 TALE repeat units.

In additional embodiments, the TALE DNA binding domain comprises about 9.5 TALE repeat units to about 14.5 TALE repeat units.

In particular embodiments, the TALE DNA binding domain binds a polynucleotide sequence in the BCL11A gene.

In particular embodiments, the TALE DNA binding domain binds the polynucleotide sequence set forth in SEQ ID NO: 26.

In certain embodiments, the polypeptide binds and cleaves the polynucleotide sequence set forth in SEQ ID NO: 27.

In certain embodiments, the zinc finger DNA binding domain comprises 2, 3, 4, 5, 6, 7, or 8 zinc finger motifs.

In further embodiments, the polypeptide further comprises a peptide linker and an end-processing enzyme or biologically active fragment thereof.

In some embodiments, the polypeptide further comprises a viral self-cleaving 2A peptide and an end-processing enzyme or biologically active fragment thereof.

In particular embodiments, the end-processing enzyme or biologically active fragment thereof has 5′-3′ exonuclease, 5′-3′ alkaline exonuclease, 3′-5′ exonuclease, 5′ flap endonuclease, helicase, template-dependent DNA polymerase or template-independent DNA polymerase activity.

In certain embodiments, the polypeptide comprises the amino acid sequence set forth in any one of SEQ ID NOs: 20-21, or a biologically active fragment thereof.

In further embodiments, the polypeptide comprises the amino acid sequence set forth in SEQ ID NO: 20, or a biologically active fragment thereof.

In particular embodiments, the polypeptide comprises the amino acid sequence set forth in SEQ ID NO: 21, or a biologically active fragment thereof.

In certain embodiments, the end-processing enzyme comprises Trex2 or a biologically active fragment thereof.

In certain embodiments, the polypeptide comprises the amino acid sequence set forth in any one of SEQ ID NOs: 22-23, or a biologically active fragment thereof.

In further embodiments, the polypeptide comprises the amino acid sequence set forth in SEQ ID NO: 22, or a biologically active fragment thereof.

In particular embodiments, the polypeptide comprises the amino acid sequence set forth in SEQ ID NO: 23, or a biologically active fragment thereof.

In further embodiments, the polypeptide cleaves the human BCL11A gene at the polynucleotide sequence set forth in SEQ ID NO: 25 or SEQ ID NO: 27.

In various embodiments, the present disclosure contemplates, in part, a polynucleotide encoding a polypeptide contemplated herein.

In particular embodiments, the present disclosure contemplates, in part, an mRNA encoding a polypeptide contemplated herein.

In particular embodiments, the mRNA comprises the sequence set forth in any one of SEQ ID NOs: 36-37.

In certain embodiments, the present disclosure contemplates, in part, a cDNA encoding a polypeptide contemplated herein.

In additional embodiments, the present disclosure contemplates, in part, a vector comprising a polynucleotide encoding a polypeptide contemplated herein.

In further embodiments, the present disclosure contemplates, in part, a cell comprising a polypeptide contemplated herein.

In various embodiments, the present disclosure contemplates, in part, a cell comprising a polynucleotide encoding a polypeptide contemplated herein.

In particular embodiments, the present disclosure contemplates, in part, a cell comprising a vector contemplated herein.

In various embodiments, the present disclosure contemplates, in part, a cell comprising one or more genome modifications introduced by a polypeptide contemplated herein.

In certain embodiments, the cell is a hematopoietic cell.

In particular embodiments, the cell is a hematopoietic stem or progenitor cell.

In some embodiments, the cell is a CD34⁺ cell.

In particular embodiments, the cell is a CD133⁺ cell.

In various embodiments, the present disclosure contemplates, in part, a composition comprising a genome edited cell contemplated herein.

In various embodiments, the present disclosure contemplates, in part, a composition comprising a genome edited cell contemplated herein and a physiologically acceptable carrier.

In particular embodiments, the present disclosure contemplates, in part, a method of editing a BCL11A gene in a population of cells comprising: introducing a polynucleotide encoding a polypeptide contemplated herein into the cell, wherein expression of the polypeptide creates a double strand break at a target site in a BCL11A gene.

In various embodiments, the present disclosure contemplates, in part, a method of editing a BCL11A gene in a population of cells comprising: introducing a polynucleotide encoding a polypeptide contemplated herein into the cell, wherein expression of the polypeptide creates a double strand break at a target site in a BCL11A gene, wherein the break is repaired by non-homologous end joining (NHEJ).

In particular embodiments, the present disclosure contemplates, in part, a method of editing a BCL11A gene in a population of cells comprising: introducing a polynucleotide encoding a polypeptide contemplated herein and a donor repair template into the cell, wherein expression of the polypeptide creates a double strand break at a target site in a BCL11A gene and the donor repair template is incorporated into the BCL11A gene by homology directed repair (HDR) at the site of the double-strand break (DSB).

In certain embodiments, the cell is a hematopoietic cell.

In further embodiments, the cell is a hematopoietic stem or progenitor cell.

In some embodiments, the cell is a CD34⁺ cell.

In particular embodiments, the cell is a CD133⁺ cell.

In further embodiments, the polynucleotide encoding the polypeptide is an mRNA.

In particular embodiments, a polynucleotide encoding a 5′-3′ exonuclease is introduced into the cell.

In certain embodiments, a polynucleotide encoding Trex2 or a biologically active fragment thereof is introduced into the cell.

In additional embodiments, the donor repair template comprises a 5′ homology arm homologous to a BCL11A gene sequence 5′ of the DSB and a 3′ homology arm homologous to a BCL11A gene sequence 3′ of the DSB.

In some embodiments, the lengths of the 5′ and 3′ homology arms are independently selected from about 100 bp to about 2500 bp.

In additional embodiments, the lengths of the 5′ and 3′ homology arms are independently selected from about 600 bp to about 1500 bp.

In some embodiments, the 5′-homology arm is about 1500 bp and the 3′ homology arm is about 1000 bp.

In further embodiments, the 5′-homology arm is about 600 bp and the 3′ homology arm is about 600 bp.

In some embodiments, a viral vector is used to introduce the donor repair template into the cell.

In additional embodiments, the viral vector is a recombinant adeno-associated viral vector (rAAV) or a retrovirus.

In particular embodiments, the rAAV has one or more ITRs from AAV2.

In further embodiments, the rAAV has a serotype selected from the group consisting of: AAV1, AAV2, AAV3, AAV4, AAV5, AAV6, AAV7, AAV8, AAV9, and AAV10.

In certain embodiments, the rAAV has an AAV2 or AAV6 serotype.

In further embodiments, the retrovirus is a lentivirus.

In some embodiments, the lentivirus is an integrase deficient lentivirus (IDLV).

In various embodiments, the present disclosure contemplates, in part, a method of treating, preventing, or ameliorating at least one symptom of a hemoglobinopathy, or condition associated therewith, comprising administering to the subject an effective amount of a composition contemplated herein.

In particular embodiments, the subject has a β-globin genotype selected from the group consisting of: β^{E}/β⁰, β^{C}/β⁰, β⁰/β⁰, β^{E}/β^{E}, β^{C}/β⁺, β^{E}/β⁺, β⁰/β⁺, β⁺/β⁺, β^{C}/β^{C}, β^{E}/β^{S}, β⁰/β^{S}, β^{C}/β^{S}, β⁺/β^{S} or β^{S}/β^{S}.

In certain embodiments, the amount of the composition is effective to decrease blood transfusions in the subject.

In various embodiments, the present disclosure contemplates, in part, a method of treating, preventing, or ameliorating at least one symptom of a thalassemia, or condition associated therewith, comprising administering to the subject an effective amount of a composition contemplated herein.

In some embodiments, the subject has an α-thalassemia or condition associated therewith.

In particular embodiments, the subject has a β-thalassemia or condition associated therewith.

In certain embodiments, the subject has a β-globin genotype selected from the group consisting of: β^{E}/β⁰, β^{C}/β⁰, β⁰/β⁰, β^{C}/β^{C}, β^{E}/β^{E}, β^{E}/β⁺, β^{C}/β^{E}, β^{C}/β⁺, β⁰/β⁺, or β⁺/β⁺.

In various embodiments, the present disclosure contemplates, in part, a method of treating, preventing, or ameliorating at least one symptom of a sickle cell disease, or condition associated therewith, comprising administering to the subject an effective amount of a composition contemplated herein.

In particular embodiments, the subject has a β-globin genotype selected from the group consisting of: β^{E}/β^{S}, β⁰/β^{S}, β^{C}/β^{S}, β⁺/β^{S} or β^{S}/β^{S}.

In various embodiments, the present disclosure contemplates, in part, a method of increasing the amount of γ-globin in a subject comprising administering to the subject an effective amount of a composition contemplated herein.

In various embodiments, the present disclosure contemplates, in part, a method of increasing the amount of fetal hemoglobin (HbF) in a subject comprising administering to the subject an effective amount of a composition contemplated herein.

In particular embodiments, the subject has a hemoglobinopathy.

In some embodiments, the subject has an α-thalassemia or condition associated therewith.

In further embodiments, the subject has a β-thalassemia or condition associated therewith.

In particular embodiments, the subject has a β-globin genotype selected from the group consisting of: β^{E}/β⁰, β^{C}/β⁰, β^{C}/β^{C}, β^{E}/β^{E}, β^{E}/β⁺, β^{C}/β^{E}, β^{C}/β⁺, β⁰/β⁺, or β⁺/β⁺.

In certain embodiments, the subject has a sickle cell disease, or condition associated therewith.

In particular embodiments, the subject has a β-globin genotype selected from the group consisting of: β^{E}/β^{S}, β⁰/β^{S}, β^{C}/β^{S}, β⁺/β^{S} or β^{S}/β^{S}.

### BRIEF DESCRIPTION OF SEVERAL VIEWS OF THE DRAWINGS

FIG. 1 shows the human BCL11A gene, with alternative splicing isoforms depicted, and the location of the GATA-1 binding motif (SEQ ID NOS: 77 and 78) and a reprogrammed homing endonuclease target site within a DNase hypersensitive site (DHS) located ˜58 kb downstream of the transcription start site.

FIG. 2A shows that the native homing endonuclease I-SmaMI cleaves a DNA target comprising TTAT as the central-4 sequence (SEQ ID NO:30).

FIG. 2B shows that an I-OnuI homing endonuclease reprogrammed target the CCR5 gene is capable of cleaving a TTAT central-4, while retaining its natural central-4 cleavage specificity.

FIG. 3 shows reprogramming of the I-OnuI N-terminal domain (NTD) and C-terminal domain (CTD) against chimeric “half-sites” through three rounds of sorting, followed by fusion of the reprogrammed domains to isolate a fully reprogrammed I-OnuI homing endonuclease that cleaves the target site.

FIG. 4A shows the initial screening of I-OnuI derived homing endonuclease variants for activity against a BCL11A target site in a chromosomal reporter assay.

FIG. 4B shows the refinement of the initially derived I-OnuI derived homing endonuclease BCL11A.A4 to achieve a more active variant, BCL11A-B4A3.

FIG. 4C shows a comparison of the catalytic activity of BCL11A.A4 and BCL11A-B4A3 for the BCL11A target sequence.

FIG. 5 shows an alignment of BCL11A.A4 (SEQ ID NO:80) and BCL11A-B4A3 (SEQ ID NO:81) homing endonucleases compared to the wild type I-OnuI homing endonucleases (SEQ ID NO:79), highlighting non-identical positions.

FIG. 6A shows that the BCL11A-B4A3 homing endonuclease has sub-nanomolar affinity properties as measured using a yeast surface display based substrate titration assay.

FIG. 6B shows the how varying the bases of the target sequence at each position affects target cleavage specificity.

FIG. 7 shows the comprehensive central-4 specificity profile of the BCL11A-B4A3 homing endonuclease, demonstrating retention of a high degree of overall selectivity amongst a slightly shifted spectrum of tolerated central-4 sequences that includes TTAT.

FIG. 8A shows a schematic of a BCL11A megaTAL that targets the BCL11A gene (SEQ ID NOS: 82 and 83).

FIG. 8B shows a TIDE analysis of BCL11A megaTAL editing of the target sequence in the BCL11A gene in primary human CD34+ hematopoietic stem cells.

FIG. 8C shows a PCR-based analysis of BCL11A megaTAL editing of the target sequence in the BCL11A gene in editing primary human CD34+ hematopoietic stem cells.

FIG. 8D shows a single colony sequencing analysis of BCL11A megaTAL editing of the target sequence (SEQ ID NOS: 84-104) in the BCL11A gene in primary human CD34+ hematopoietic stem cells.

FIG. 8E shows results from additional experiments for BCL11A megaTAL editing of the target sequence in the BCL11A gene in primary human CD34+ hematopoietic stem cells.

FIG. 9A shows a schematic of a donor repair template comprising homology arms flanking the BCL11A target sequence and a fluorescent reporter gene embedded between two homology arms.

FIG. 9B shows that introduction of a BCL11A megaTAL into CD34+ cells and transduction of the cells with an AAV6 genome comprising a donor repair template carrying a transgene cassette embedded between two homology arms, results in a high rate of targeted insertion of the cassette at the target site in the BCL11A gene.

FIG. 10A shows that introduction of a BCL11A megaTAL into CD34+ cells and transduction of the cells with an AAV6 genome comprising a donor repair template does not substantially alter the erythroid differentiation capacity of human CD34+ cells.

FIG. 10B shows a tabular representation of the data shown in FIG. 10A.

FIG. 11A is a representative flow cytometry analysis showing that primary human CD34+ hematopoietic stem cell populations treated with a BCL11A megaTAL upregulate fetal hemoglobin when differentiated to erythroid lineage cells.

FIG. 11B is a representative HPLC analysis showing that primary human CD34+ hematopoietic stem cell populations treated with a BCL11A megaTAL upregulate fetal hemoglobin when differentiated to erythroid lineage cells.

FIG. 12 shows colony formation is unaffected in primary human CD34+ hematopoietic stem cell populations treated with a BCL11A megaTAL.

FIG. 13 shows the editing rates of human CD34+ cells electroporated without mRNA or with mRNA encoding a CCR5 megaTAL, a CCR5 megaTAL-Trex2 fusion protein, a BCL11A megaTAL, or a BCL11A megaTAL-Trex2 fusion protein.

FIG. 14 shows the level of HbF production from human CD34+ cells electroporated without mRNA or with mRNA encoding a CCR5 megaTAL, a CCR5 megaTAL-Trex2 fusion protein, a BCL11A megaTAL, or a BCL11A megaTAL-Trex2 fusion protein.

FIG. 15 shows that primary human CD34+ hematopoietic stem cell populations treated with a BCL11A megaTAL stably engraft in immunodeficient mice with minimal diminution of edited cells.

FIG. 16 shows the level of HbF production from a human CD34⁺ cell grafts and from 4 month bone marrow from transplanted NSG mice with the grafts. Human CD34+ cells electroporated without mRNA or with mRNA encoding a CCR5 megaTAL, a CCR5 megaTAL-Trex2 fusion protein, a BCL11A megaTAL, or a BCL11A megaTAL-Trex2 fusion protein.

### BRIEF DESCRIPTION OF THE SEQUENCE IDENTIFIERS

SEQ ID NO: 1 is an amino acid sequence of a wild type I-OnuI LAGLIDADG homing endonuclease (LHE).

SEQ ID NO: 2 is an amino acid sequence of a wild type I-OnuI LHE.

SEQ ID NO: 3 is an amino acid sequence of a biologically active fragment of a wild-type I-OnuI LHE.

SEQ ID NO: 4 is an amino acid sequence of a biologically active fragment of a wild-type I-OnuI LHE.

SEQ ID NO: 5 is an amino acid sequence of a biologically active fragment of a wild-type I-OnuI LHE.

SEQ ID NOs: 6-19 is an amino acid sequence of an I-OnuI LHE variant reprogrammed to bind and cleave a target site in the human BCL11A gene.

SEQ ID NO: 20 is an amino acid sequence of a megaTAL that binds and cleaves a target site in the human BCL11A gene.

SEQ ID NO: 21 is an amino acid sequence of a megaTAL that binds and cleaves a target site in the human BCL11A gene.

SEQ ID NO: 22 is an amino acid sequence of a megaTAL-Trex2 fusion protein that binds and cleaves a target site in the human BCL11A gene.

SEQ ID NO: 23 is an amino acid sequence of a megaTAL-Trex2 fusion protein that binds and cleaves a target site in the human BCL11A gene.

SEQ ID NO: 24 is a polynucleotide comprising a GATA-1 motif in DNA hypersensitive site 58 of the human BCL11A gene.

SEQ ID NO: 25 is an I-OnuI LHE variant target site in the human BCL11A gene.

SEQ ID NO: 26 is a TALE DNA binding domain target site in the human BCL11A gene.

SEQ ID NO: 27 is a megaTAL target site in the human BCL11A gene.

SEQ ID NO: 28 is an I-OnuI LHE variant N-terminal domain target site.

SEQ ID NO: 29 is an I-OnuI LHE variant C-terminal domain target site.

SEQ ID NO: 30 is an I-SmaMI LHE target site.

SEQ ID NO: 31 is an I-OnuI LHE variant target site in the human CCR5 gene.

SEQ ID NO: 32 is a polynucleotide sequence of an I-OnuI LHE variant surface display plasmid for an I-OnuI LHE variant that binds and cleaves a target site in the human CCR5 gene.

SEQ ID NO: 33 is a polynucleotide sequence for a central 4 array for an I-OnuI LHE variant that binds and cleaves a target site in the human CCR5 gene.

SEQ ID NO: 34 is a polynucleotide sequence of an I-OnuI LHE variant surface display plasmid for an I-OnuI LHE variant that binds and cleaves a target site in the human BCL11A gene.

SEQ ID NO: 35 is a polynucleotide sequence for a central 4 array for an I-OnuI LHE variant that binds and cleaves a target site in the human BCL11A gene.

SEQ ID NO: 36 is an mRNA sequence encoding a megaTAL that cleaves the human BCL11A gene.

SEQ ID NO: 37 is an mRNA sequence encoding a megaTAL-Trex2 fusion that cleaves the human BCL11A gene.

SEQ ID NO: 38 is an mRNA sequence encoding murine Trex2.

SEQ ID NO: 39 is an amino acid sequence encoding murine Trex2.

SEQ ID NOs: 40-50 set forth the amino acid sequences of various linkers.

SEQ ID NOs: 51-75 set forth the amino acid sequences of protease cleavage sites and self-cleaving polypeptide cleavage sites.

In the foregoing sequences, X, if present, refers to any amino acid or the absence of an amino acid.

### DETAILED DESCRIPTION

### A. Overview

The present disclosure generally relates to, in part, improved genome editing compositions and methods of use thereof. Without wishing to be bound by any particular theory, the genome editing compositions contemplated herein are used to increase the amount of fetal hemoglobin in a cell to treat, prevent, or ameliorates symptoms associated with various hemoglobinopathies. Thus, the compositions contemplated herein offer a potentially curative solution to subjects that have a hemoglobinopathy.

Normal adult hemoglobin comprises a tetrameric complex of two alpha-(α) globin proteins and two beta- (β-) globin proteins. In development, the fetus produces fetal hemoglobin (HbF), which comprises two gamma- (γ) globin proteins instead of the two β-globin proteins. At some point during perinatal development, a “globin switch” occurs; erythrocytes down-regulate γ-globin expression and switch to predominantly producing β-globin. This switch results primarily from decreased transcription of the γ-globin genes and increased transcription of β-globin genes. GATA binding protein-1 (GATA-1) is a transcription factor that influences globin switch. GATA-1 directly transactivates β-globin gene expression and indirectly represses or suppresses γ-globin gene expression through transactivation of BCL11A expression. Pharmacologic or genetic manipulation of the switch represents an attractive therapeutic strategy for patients who suffer from 3-thalassemia or sickle-cell disease due to mutations in the 3-globin gene.

In various embodiments, nuclease variants that disrupt BCL11A gene function and/or expression in erythroid cells, genome editing compositions, genetically modified cells, and methods of use thereof are contemplated. BCL11A expression in the erythroid compartment is heavily dependent on an erythroid enhancer comprising a consensus GATA-1 binding motif WGATAA (SEQ ID NO: 24) in the second intron of the BCL11A gene. Without wishing to be bound by any particular theory, it is contemplated that reducing or eliminating BCL11A expression in erythroid cells through genome editing of the GATA-1 binding site would result in the reactivation or derepression of γ-globin gene expression and a decrease in β-globin gene expression, and thereby increase HbF expression to effectively treat and/or ameliorate one or more symptoms associated with subjects that have a hemoglobinopathy.

Genome editing methods contemplated in various embodiments comprise nuclease variants, designed to bind and cleave a transcription factor binding site in the B Cell CLL/Lymphoma 11A gene (BCL11A). The nuclease variants contemplated in particular embodiments, can be used to introduce a double-strand break in a target polynucleotide sequence, which may be repaired by non-homologous end joining (NHEJ) in the absence of a polynucleotide template, e.g., a donor repair template, or by homology directed repair (HDR), i.e., homologous recombination, in the presence of a donor repair template. Nuclease variants contemplated in certain embodiments, can also be designed as nickases, which generate single-stranded DNA breaks that can be repaired using the cell's base-excision-repair (BER) machinery or homologous recombination in the presence of a donor repair template. NHEJ is an error-prone process that frequently results in the formation of small insertions and deletions that disrupt gene function. Homologous recombination requires homologous DNA as a template for repair and can be leveraged to create a limitless variety of modifications specified by the introduction of donor DNA containing the desired sequence at the target site, flanked on either side by sequences bearing homology to regions flanking the target site.

In one preferred embodiment, the genome editing compositions contemplated herein comprise homing endonuclease variants or megaTALs that target the human BCL11A gene.

In various embodiments, wherein a DNA break is generated in an erythroid specific enhancer in the BCL11A gene, NHEJ of the ends of the cleaved genomic sequence may result in a cell with decreased BCL11A expression, and preferably an erythroid cell that lacks or substantially lacks functional BCL11A expression, e.g., lacks the ability to repress or suppress γ-globin gene transcription and lacks the ability to transactivate β-globin gene transcription.

In various other embodiments, wherein a donor template for repair of the cleaved BCL11A genomic sequence is provided, the DSB is repaired with the sequence of the template by homologous recombination at the DNA break-site. In preferred embodiments, the repair template comprises a polynucleotide sequence that is different from a targeted genomic sequence.

In one preferred embodiment, the genome editing compositions contemplated herein comprise nuclease variants and one or more end-processing enzymes to increase NHEJ or HDR efficiency.

In one preferred embodiment, the genome editing compositions contemplated herein comprise a homing endonuclease variant or megaTAL that targets a human BCL11A gene and an end-processing enzyme, e.g., Trex2.

In various embodiments, genome edited cells are contemplated. The genome edited cells comprise decreased endogenous BCL11A expression in erythroid cell lineages. The genome edited erythroid cells comprise increased γ-globin expression and decreased β-globin expression.

Accordingly, the methods and compositions contemplated herein represent a quantum improvement compared to existing gene editing strategies for the treatment of hemoglobinopathies.

The practice of the particular embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, 9%, 8%, ±7%, ±6%, ±5%, ±4%, ±3%, ±2%, or ±1% about a reference quantity, level, value, number, frequency, percentage, dimension, size, amount, weight or length.

In one embodiment, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

The term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured or modulated in a laboratory apparatus, usually under sterile conditions, and typically for a few hours or up to about 24 hours, but including up to 48 or 72 hours, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo.

The term “in vivo” refers generally to activities that take place inside an organism. In one embodiment, cellular genomes are engineered, edited, or modified in vivo.

By “enhance” or “promote” or “increase” or “expand” or “potentiate” refers generally to the ability of a nuclease variant, genome editing composition, or genome edited cell contemplated herein to produce, elicit, or cause a greater response (i.e., physiological response) compared to the response caused by either vehicle or control. A measurable response may include an increase in γ-globin expression, HbF expression, and/or an increase in transfusion independence, among others apparent from the understanding in the art and the description herein. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or control.

By “decrease” or “lower” or “lessen” or “reduce” or “abate” or “ablate” or “inhibit” or “dampen” refers generally to the ability of nuclease variant, genome editing composition, or genome edited cell contemplated herein to produce, elicit, or cause a lesser response (i.e., physiological response) compared to the response caused by either vehicle or control. A measurable response may include a decrease in endogenous β-globin, transfusion dependence, RBC sickling, and the like. A “decrease” or “reduced” amount is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, or control.

By “maintain,” or “preserve,” or “maintenance,” or “no change,” or “no substantial change,” or “no substantial decrease” refers generally to the ability of a nuclease variant, genome editing composition, or genome edited cell contemplated herein to produce, elicit, or cause a substantially similar or comparable physiological response (i.e., downstream effects) in as compared to the response caused by either vehicle or control. A comparable response is one that is not significantly different or measurable different from the reference response.

The terms “specific binding affinity” or “specifically binds” or “specifically bound” or “specific binding” or “specifically targets” as used herein, describe binding of one molecule to another, e.g., DNA binding domain of a polypeptide binding to DNA, at greater binding affinity than background binding. A binding domain “specifically binds” to a target site if it binds to or associates with a target site with an affinity or Kₐ (i.e., an equilibrium association constant of a particular binding interaction with units of 1/M) of, for example, greater than or equal to about 10⁵ M⁻¹. In certain embodiments, a binding domain binds to a target site with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding domains refers to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater.

Alternatively, affinity may be defined as an equilibrium dissociation constant (K_{d}) of a particular binding interaction with units of M (e.g., 10⁻⁵ M to 10⁻¹³ M, or less). Affinities of nuclease variants comprising one or more DNA binding domains for DNA target sites contemplated in particular embodiments can be readily determined using conventional techniques, e.g., yeast cell surface display, or by binding association, or displacement assays using labeled ligands.

In one embodiment, the affinity of specific binding is about 2 times greater than background binding, about 5 times greater than background binding, about 10 times greater than background binding, about 20 times greater than background binding, about 50 times greater than background binding, about 100 times greater than background binding, or about 1000 times greater than background binding or more.

The terms “selectively binds” or “selectively bound” or “selectively binding” or “selectively targets” and describe preferential binding of one molecule to a target molecule (on-target binding) in the presence of a plurality of off-target molecules. In particular embodiments, an HE or megaTAL selectively binds an on-target DNA binding site about 5, 10, 15, 20, 25, 50, 100, or 1000 times more frequently than the HE or megaTAL binds an off-target DNA target binding site.

“On-target” refers to a target site sequence.

“Off-target” refers to a sequence similar to but not identical to a target site sequence.

A “target site” or “target sequence” is a chromosomal or extrachromosomal nucleic acid sequence that defines a portion of a nucleic acid to which a binding molecule will bind and/or cleave, provided sufficient conditions for binding and/or cleavage exist. When referring to a polynucleotide sequence or SEQ ID NO. that references only one strand of a target site or target sequence, it would be understood that the target site or target sequence bound and/or cleaved by a nuclease variant is double-stranded and comprises the reference sequence and its complement. In a preferred embodiment, the target site is a sequence in the human BCL11A gene.

“Recombination” refers to a process of exchange of genetic information between two polynucleotides, including but not limited to, donor capture by non-homologous end joining (NHEJ) and homologous recombination. For the purposes of this disclosure, “homologous recombination (HR)” refers to the specialized form of such exchange that takes place, for example, during repair of double-strand breaks in cells via homology-directed repair (HDR) mechanisms. This process requires nucleotide sequence homology, uses a “donor” molecule as a template to repair a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

“NHEJ” or “non-homologous end joining” refers to the resolution of a double-strand break in the absence of a donor repair template or homologous sequence. NHEJ can result in insertions and deletions at the site of the break. NHEJ is mediated by several sub-pathways, each of which has distinct mutational consequences. The classical NHEJ pathway (cNHEJ) requires the KU/DNA-PKcs/Lig4/XRCC4 complex, ligates ends back together with minimal processing and often leads to precise repair of the break. Alternative NHEJ pathways (altNHEJ) also are active in resolving dsDNA breaks, but these pathways are considerably more mutagenic and often result in imprecise repair of the break marked by insertions and deletions. While not wishing to be bound to any particular theory, it is contemplated that modification of dsDNA breaks by end-processing enzymes, such as, for example, exonucleases, e.g., Trex2, may bias repair towards an altNHEJ pathway.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, polypeptides and nuclease variants, e.g., homing endonuclease variants, megaTALs, etc. contemplated herein are used for targeted double-stranded DNA cleavage. Endonuclease cleavage recognition sites may be on either DNA strand.

An “exogenous” molecule is a molecule that is not normally present in a cell, but that is introduced into a cell by one or more genetic, biochemical or other methods. Exemplary exogenous molecules include, but are not limited to small organic molecules, protein, nucleic acid, carbohydrate, lipid, glycoprotein, lipoprotein, polysaccharide, any modified derivative of the above molecules, or any complex comprising one or more of the above molecules. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, biopolymer nanoparticle, calcium phosphate co-precipitation, DEAE-dextran-mediated transfer and viral vector-mediated transfer.

An “endogenous” molecule is one that is normally present in a particular cell at a particular developmental stage under particular environmental conditions. Additional endogenous molecules can include proteins, for example, endogenous globins.

A “gene,” refers to a DNA region encoding a gene product, as well as all DNA regions which regulate the production of the gene product, whether or not such regulatory sequences are adjacent to coding and/or transcribed sequences. A gene includes, but is not limited to, promoter sequences, enhancers, silencers, insulators, boundary elements, terminators, polyadenylation sequences, post-transcription response elements, translational regulatory sequences such as ribosome binding sites and internal ribosome entry sites, replication origins, matrix attachment sites, and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

As used herein, the term “genetically engineered” or “genetically modified” refers to the chromosomal or extrachromosomal addition of extra genetic material in the form of DNA or RNA to the total genetic material in a cell. Genetic modifications may be targeted or non-targeted to a particular site in a cell's genome. In one embodiment, genetic modification is site specific. In one embodiment, genetic modification is not site specific.

As used herein, the term “genome editing” refers to the substitution, deletion, and/or introduction of genetic material at a target site in the cell's genome, which restores, corrects, disrupts, and/or modifies expression of a gene or gene product. Genome editing contemplated in particular embodiments comprises introducing one or more nuclease variants into a cell to generate DNA lesions at or proximal to a target site in the cell's genome, optionally in the presence of a donor repair template.

As used herein, the term “gene therapy” refers to the introduction of extra genetic material into the total genetic material in a cell that restores, corrects, or modifies expression of a gene or gene product, or for the purpose of expressing a therapeutic polypeptide. In particular embodiments, introduction of genetic material into the cell's genome by genome editing that restores, corrects, disrupts, or modifies expression of a gene or gene product, or for the purpose of expressing a therapeutic polypeptide is considered gene therapy.

### C. Nuclease Variants

Nuclease variants contemplated in particular embodiments herein that are suitable for genome editing a target site in the BCL11A gene and comprise one or more DNA binding domains and one or more DNA cleavage domains (e.g., one or more endonuclease and/or exonuclease domains), and optionally, one or more linkers contemplated herein. The terms “reprogrammed nuclease,” “engineered nuclease,” or “nuclease variant” are used interchangeably and refer to a nuclease comprising one or more DNA binding domains and one or more DNA cleavage domains, wherein the nuclease has been designed and/or modified from a parental or naturally occurring nuclease, to bind and cleave a double-stranded DNA target sequence in a BCL11A gene, preferably in a GATA-1 binding site in the BCL11A gene, more preferably in a consensus GATA-1 binding site in the second intron of the BCL11A gene, and even more preferably in a target site set forth in SEQ ID NO: 25 (the complement of which includes the Consensus GATA-1 motif WGATAR). The nuclease variant may be designed and/or modified from a naturally occurring nuclease or from a previous nuclease variant. Nuclease variants contemplated in particular embodiments may further comprise one or more additional functional domains, e.g., an end-processing enzymatic domain of an end-processing enzyme that exhibits 5′-3′ exonuclease, 5′-3′ alkaline exonuclease, 3′-5′-exonuclease (e.g., Trex2), 5′ flap endonuclease, helicase, template-dependent DNA polymerase or template-independent DNA polymerase activity.

Illustrative examples of nuclease variants that bind and cleave a target sequence in the BCL11A gene include, but are not limited to homing endonuclease variants (meganuclease variants) and megaTALs.

1. Homing Endonuclease (Meganuclease) Variants

In various embodiments, a homing endonuclease or meganuclease is reprogrammed to introduce double-strand breaks (DSBs) in an erythroid specific enhancer in the BCL11A gene, preferably in a GATA-1 binding site in the BCL11A gene, more preferably in a consensus GATA-1 binding site in the second intron of the BCL11A gene, and even more preferably in a target site set forth in SEQ ID NO: 25 (the complement of which includes the Consensus GATA-1 motif WGATAR). “Homing endonuclease” and “meganuclease” are used interchangeably and refer to naturally-occurring nucleases that recognize 12-45 base-pair cleavage sites and are commonly grouped into five families based on sequence and structure motifs: LAGLIDADG, GIY-YIG, HNH, His-Cys box, and PD-(D/E)XK.

A “reference homing endonuclease” or “reference meganuclease” refers to a wild type homing endonuclease or a homing endonuclease found in nature. In one embodiment, a “reference homing endonuclease” refers to a wild type homing endonuclease that has been modified to increase basal activity.

An “engineered homing endonuclease,” “reprogrammed homing endonuclease,” “homing endonuclease variant,” “engineered meganuclease,” “reprogrammed meganuclease,” or “meganuclease variant” refers to a homing endonuclease comprising one or more DNA binding domains and one or more DNA cleavage domains, wherein the homing endonuclease has been designed and/or modified from a parental or naturally occurring homing endonuclease, to bind and cleave a DNA target sequence in a BCL11A gene. The homing endonuclease variant may be designed and/or modified from a naturally occurring homing endonuclease or from another homing endonuclease variant. Homing endonuclease variants contemplated in particular embodiments may further comprise one or more additional functional domains, e.g., an end-processing enzymatic domain of an end-processing enzyme that exhibits 5′-3′ exonuclease, 5′-3′ alkaline exonuclease, 3′-5′ exonuclease (e.g., Trex2), 5′ flap endonuclease, helicase, template dependent DNA polymerase or template-independent DNA polymerases activity.

Homing endonuclease (HE) variants do not exist in nature and can be obtained by recombinant DNA technology or by random mutagenesis. HE variants may be obtained by making one or more amino acid alterations, e.g., mutating, substituting, adding, or deleting one or more amino acids, in a naturally occurring HE or HE variant. In particular embodiments, a HE variant comprises one or more amino acid alterations to the DNA recognition interface.

HE variants contemplated in particular embodiments may further comprise one or more linkers and/or additional functional domains, e.g., an end-processing enzymatic domain of an end-processing enzyme that exhibits 5′-3′ exonuclease, 5′-3′ alkaline exonuclease, 3′-5′ exonuclease (e.g., Trex2), 5′ flap endonuclease, helicase, template-dependent DNA polymerase or template-independent DNA polymerases activity. In particular embodiments, HE variants are introduced into a T cell with an end-processing enzyme that exhibits 5′-3′ exonuclease, 5′-3′ alkaline exonuclease, 3′-5′ exonuclease (e.g., Trex2), 5′ flap endonuclease, helicase, template-dependent DNA polymerase or template-independent DNA polymerases activity. The HE variant and 3′ processing enzyme may be introduced separately, e.g., in different vectors or separate mRNAs, or together, e.g., as a fusion protein, or in a polycistronic construct separated by a viral self-cleaving peptide or an IRES element.

A “DNA recognition interface” refers to the HE amino acid residues that interact with nucleic acid target bases as well as those residues that are adjacent. For each HE, the DNA recognition interface comprises an extensive network of side chain-to-side chain and side chain-to-DNA contacts, most of which is necessarily unique to recognize a particular nucleic acid target sequence. Thus, the amino acid sequence of the DNA recognition interface corresponding to a particular nucleic acid sequence varies significantly and is a feature of any natural or HE variant. By way of non-limiting example, a HE variant contemplated in particular embodiments may be derived by constructing libraries of HE variants in which one or more amino acid residues localized in the DNA recognition interface of the natural HE (or a previously generated HE variant) are varied. The libraries may be screened for target cleavage activity against each predicted BCL11A target site using cleavage assays (see e.g., Jarjour et al., 2009. Nuc. Acids Res. 37(20): 6871-6880).

LAGLIDADG homing endonucleases (LHE) are the most well studied family of homing endonucleases, are primarily encoded in archaea and in organellar DNA in green algae and fungi, and display the highest overall DNA recognition specificity. LHEs comprise one or two LAGLIDADG catalytic motifs per protein chain and function as homodimers or single chain monomers, respectively. Structural studies of LAGLIDADG proteins identified a highly conserved core structure (Stoddard 2005), characterized by an αββαββα fold, with the LAGLIDADG motif belonging to the first helix of this fold. The highly efficient and specific cleavage of LHEs represents a protein scaffold to derive novel, highly specific endonucleases. However, engineering LHEs to bind and cleave a non-natural or non-canonical target site requires selection of the appropriate LHE scaffold, examination of the target locus, selection of putative target sites, and extensive alteration of the LHE to alter its DNA contact points and cleavage specificity, at up to two-thirds of the base-pair positions in a target site.

In one embodiment, LHEs from which reprogrammed LHEs or LHE variants may be designed include, but are not limited to I-CreI and I-SceI.

Illustrative examples of LHEs from which reprogrammed LHEs or LHE variants may be designed include, but are not limited to I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, and I-Vdil41I.

In one embodiment, the reprogrammed LHE or LHE variant is selected from the group consisting of: an I-CpaMI variant, an I-HjeMI variant, an I-OnuI variant, an I-PanMI variant, and an I-SmaMI variant.

In one embodiment, the reprogrammed LHE or LHE variant is an I-OnuI variant. See e.g., SEQ ID NOs: 6-19.

In one embodiment, reprogrammed I-OnuI LHEs or I-OnuI variants targeting the BCL11A gene were generated from a natural I-OnuI or biologically active fragment thereof (SEQ ID NOs: 1-5). In a preferred embodiment, reprogrammed I-OnuI LHEs or I-OnuI variants targeting the human BCL11A gene were generated from an existing I-OnuI variant. In one embodiment, reprogrammed I-OnuI LHEs were generated against a human BCL11A gene target site set forth in SEQ ID NO: 25.

In a particular embodiment, the reprogrammed I-OnuI LHE or I-OnuI variant that binds and cleaves the human BCL11A gene comprises one or more amino acid substitutions in the DNA recognition interface. In particular embodiments, the I-OnuI LHE that binds and cleaves the human BCL11A gene comprises at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the DNA recognition interface of I-OnuI (Taekuchi et al. 2011. Proc Natl Acad Sci U.S.A. 2011 Aug. 9; 108(32): 13077-13082) or an I-OnuI LHE variant as set forth in SEQ ID NOs: 6-19, or further variants thereof.

In one embodiment, the I-OnuI LHE that binds and cleaves the human BCL11A gene comprises at least 70%, more preferably at least 80%, more preferably at least 85%, more preferably at least 90%, more preferably at least 95%, more preferably at least 97%, more preferably at least 99% sequence identity with the DNA recognition interface of I-OnuI (Taekuchi et al. 2011. Proc Natl Acad Sci U.S.A. 2011 Aug. 9; 108(32): 13077-13082) or an I-OnuI LHE variant as set forth in SEQ ID NOs: 6-19, or further variants thereof.

In a particular embodiment, an I-OnuI LHE variant that binds and cleaves the human BCL11A gene comprises one or more amino acid substitutions or modifications in the DNA recognition interface of an I-OnuI as set forth in any one of SEQ ID NOs: 1-19.

In a particular embodiment, an I-OnuI LHE variant that binds and cleaves the human BCL11A gene comprises one or more amino acid substitutions or modifications in the DNA recognition interface, particularly in the subdomains situated from positions 24-50, 68 to 82, 180 to 203 and 223 to 240 of I-OnuI (SEQ ID NOs: 1-5) an I-OnuI variant as set forth in SEQ ID NOs: 6-19, or further variants thereof.

In a particular embodiment, an I-OnuI LHE that binds and cleaves the human BCL11A gene comprises one or more amino acid substitutions or modifications in the DNA recognition interface at amino acid positions selected from the group consisting of: 19, 24, 26, 28, 30, 32, 34, 35, 36, 37, 38, 40, 42, 44, 46, 48, 68, 70, 72, 75, 76 77, 78, 80, 82, 168, 180, 182, 184, 186, 188, 189, 190, 191, 192, 193, 195, 197, 199, 201, 203, 223, 225, 227, 229, 231, 232, 234, 236, 238, and 240 of I-OnuI (SEQ ID NOs: 1-5) or an I-OnuI variant as set forth in SEQ ID NOs: 6-19, or further variants thereof.

In a particular embodiment, an I-OnuI LHE that binds and cleaves the human BCL11A gene comprises 5, 10, 15, 20, 25, 30, 35, or 40 or more amino acid substitutions or modifications in the DNA recognition interface, particularly in the subdomains situated from positions 24-50, 68 to 82, 180 to 203 and 223 to 240 of I-OnuI (SEQ ID NOs: 1-5) or an I-OnuI variant as set forth in SEQ ID NOs: 6-19, or further variants thereof.

In a particular embodiment, an I-OnuI LHE variant that binds and cleaves the human BCL11A gene comprises 5, 10, 15, 20, 25, 30, 35, or 40 or more amino acid substitutions or modifications in the DNA recognition interface at amino acid positions selected from the group consisting of: 19, 24, 26, 28, 30, 32, 34, 35, 36, 37, 38, 40, 42, 44, 46, 48, 68, 70, 72, 75, 76 77, 78, 80, 82, 168, 180, 182, 184, 186, 188, 189, 190, 191, 192, 193, 195, 197, 199, 201, 203, 223, 225, 227, 229, 231, 232, 234, 236, 238, and 240 of I-OnuI SEQ ID NOs: 1-5) or an I-OnuI variant as set forth in SEQ ID NOs: 6-19, or further variants thereof.

In one embodiment, an I-OnuI LHE variant that binds and cleaves the human BCL11A gene comprises one or more amino acid substitutions or modifications at additional positions situated anywhere within the entire I-OnuI sequence. The residues which may be substituted and/or modified include but are not limited to amino acids that contact the nucleic acid target or that interact with the nucleic acid backbone or with the nucleotide bases, directly or via a water molecule. In one non-limiting example a I-OnuI LHE variant contemplated herein that binds and cleaves the human BCL11A gene comprises one or more substitutions and/or modifications, preferably at least 5, preferably at least 10, preferably at least 15, preferably at least 20, more preferably at least 25, more preferably at least 30, even more preferably at least 35, or even more preferably at least 40 in at least one position selected from the position group consisting of positions: 26, 28, 30, 32, 34, 35, 36, 37, 40, 41, 42, 44, 68, 70, 72, 76, 78, 80, 82, 138, 143, 159, 178, 180, 184, 186, 189, 190, 191, 192, 193, 195, 201, 203, 207, 223, 225, 227, 232, 236, 238, and 240, in reference to any one of SEQ ID NOs: 1-19.

In particular embodiments, an I-OnuI LHE variant that binds and cleaves the human BCL11A gene comprises at least 5, at least 15, preferably at least 25, more preferably at least 35, or even more preferably at least 40 or more amino acid substitutions at amino acid positions selected from the group consisting of: 26, 28, 30, 32, 34, 35, 36, 37, 40, 41, 42, 44, 48, 50, 53, 68, 70, 72, 76, 78, 80, 82, 138, 143, 159, 178, 180, 184, 186, 189, 190, 191, 192, 193, 195, 201, 203, 207, 223, 225, 227, 232, 236, 238, and 240 of an I-OnuI LHE amino acid sequence as set forth in SEQ ID NOs: 1-19, or a biologically active fragment thereof.

In further embodiments, an I-OnuI LHE variant that binds and cleaves the human BCL11A gene comprises at least 5, at least 15, preferably at least 25, more preferably at least 35, or even more preferably at least 40 or more of the following amino acid substitutions: L26V, L26R, L26Y, R28S, R28G, R30Q, R30H, N32R, N32S, N32K, N33S, K34D, K34N, S35Y, S36A, V37T, S40R, T41I, E42H, E42R, G44T, G44R, T48I, T48G, T48V, H50R, D53E, V68K, V68R, A70N, A70E, A70N, A70Q, A70L, A70S, S72A, S72T, S72V, S72M, A76L, A76H, A76R, S78Q, K80R, K80V, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E of I-OnuI (SEQ ID NOs: 1-5) or an I-OnuI variant as set forth in any one of SEQ ID NOs: 6-19, biologically active fragments thereof, and/or further variants thereof.

In certain embodiments, an I-OnuI LHE variant that binds and cleaves the human BCL11A gene comprises the following amino acid substitutions: L26V, R28S, R30Q, N32R, K34D, S35Y, S36A, V37T, S40R, T41I, E42H, G44T, V68K, A70N, S72A, A76L, S78Q, K80R, T82Y, L138M, T143N, S159P, C180S, N184R, I186R, K189N, S190V, K191N, L192A, G193R, Q195R, S201E, T203S, K207R, Y223H, K225Y, K227G, F232R, D236Q, V238R, and T240E of I-OnuI (SEQ ID NOs: 1-5) or an I-OnuI variant as set forth in any one of SEQ ID NOs: 6-19, biologically active fragments thereof, and/or further variants thereof.

In particular embodiments, an I-OnuI LHE variant that binds and cleaves the human BCL11A gene comprises the following amino acid substitutions: L26V, R28S, R30Q, N32R, K34D, S35Y, S36A, V37T, S40R, T41I, E42H, G44T, V68K, A70N, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E of I-OnuI (SEQ ID NOs: 1-5) or an I-OnuI variant as set forth in any one of SEQ ID NOs: 6-19, biologically active fragments thereof, and/or further variants thereof.

In some embodiments, an I-OnuI LHE variant that binds and cleaves the human BCL11A gene comprises the following amino acid substitutions: L26V, R30Q, N32S, K34D, S35Y, S36A, V37T, S40R, T41I, E42H, G44T, V68K, A70N, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E of I-OnuI (SEQ ID NOs: 1-5) or an I-OnuI variant as set forth in any one of SEQ ID NOs: 6-19, biologically active fragments thereof, and/or further variants thereof.

In certain embodiments, an I-OnuI LHE variant that binds and cleaves the human BCL11A gene comprises the following amino acid substitutions: L26V, R28S, R30Q, N32K, K34N, S35Y, S36A, V37T, S40R, T41I, E42H, G44T, T48I, V68K, A70N, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E of I-OnuI (SEQ ID NOs: 1-5) or an I-OnuI variant as set forth in any one of SEQ ID NOs: 6-19, biologically active fragments thereof, and/or further variants thereof.

In particular embodiments, an I-OnuI LHE variant that binds and cleaves the human BCL11A gene comprises the following amino acid substitutions: L26V, R28S, R30Q, N32R, K34D, S35Y, S36A, V37T, S40R, T41I, E42R, G44T, T48I, V68K, A70N, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E of I-OnuI (SEQ ID NOs: 1-5) or an I-OnuI variant as set forth in any one of SEQ ID NOs: 6-19, biologically active fragments thereof, and/or further variants thereof.

In additional embodiments, an I-OnuI LHE variant that binds and cleaves the human BCL11A gene comprises the following amino acid substitutions: L26V, R28G, R30Q, N32R, K34D, S35Y, S36A, V37T, S40R, T41I, E42R, G44T, H50R, V68K, A70N, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E of I-OnuI (SEQ ID NOs: 1-5) or an I-OnuI variant as set forth in any one of SEQ ID NOs: 6-19, biologically active fragments thereof, and/or further variants thereof.

In particular embodiments, an I-OnuI LHE variant that binds and cleaves the human BCL11A gene comprises the following amino acid substitutions: L26V, R28S, R30H, N32R, K34D, S35Y, S36A, V37T, S40R, T41I, E42H, G44R, V68K, A70N, S72T, A76H, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E of I-OnuI (SEQ ID NOs: 1-5) or an I-OnuI variant as set forth in any one of SEQ ID NOs: 6-19, biologically active fragments thereof, and/or further variants thereof.

In certain embodiments, an I-OnuI LHE variant that binds and cleaves the human BCL11A gene comprises the following amino acid substitutions: L26R, R28S, R30Q, N32R, K34D, S35Y, S36A, V37T, S40R, T41I, E42H, G44R, V68K, A70N, S72TA76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E of I-OnuI (SEQ ID NOs: 1-5) or an I-OnuI variant as set forth in any one of SEQ ID NOs: 6-19, biologically active fragments thereof, and/or further variants thereof.

In particular embodiments, an I-OnuI LHE variant that binds and cleaves the human BCL11A gene comprises the following amino acid substitutions: L26Y, R28S, R30Q, N32R, K34D, S35Y, S36A, V37T, S40R, T41I, E42H, G44R, D53E, V68R, A70E, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E of I-OnuI (SEQ ID NOs: 1-5) or an I-OnuI variant as set forth in any one of SEQ ID NOs: 6-19, biologically active fragments thereof, and/or further variants thereof.

In some embodiments, an I-OnuI LHE variant that binds and cleaves the human BCL11A gene comprises the following amino acid substitutions: L26V, R28S, R30Q, N32R, N33S, K34D, S35Y, S36A, V37T, S40R, T41I, E42H, G44R, D53E, V68K, A70N, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E of I-OnuI (SEQ ID NOs: 1-5) or an I-OnuI variant as set forth in any one of SEQ ID NOs: 6-19, biologically active fragments thereof, and/or further variants thereof.

In certain embodiments, an I-OnuI LHE variant that binds and cleaves the human BCL11A gene comprises the following amino acid substitutions: L26V, R28S, R30Q, N32R, N33S, K34D, S35Y, S36A, V37T, S40R, T41I, E42H, G44R, T48G, V68K, S72V, A76R, S78Q, K80V, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E of I-OnuI (SEQ ID NOs: 1-5) or an I-OnuI variant as set forth in any one of SEQ ID NOs: 6-19, biologically active fragments thereof, and/or further variants thereof.

In certain embodiments, an I-OnuI LHE variant that binds and cleaves the human BCL11A gene comprises the following amino acid substitutions: L26V, R28S, R30Q, N32R, N33S, K34D, S35Y, S36A, V37T, S40R, T41I, E42H, G44R, T48G, V68K, A70Q, S72M, A76R, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E of I-OnuI (SEQ ID NOs: 1-5) or an I-OnuI variant as set forth in any one of SEQ ID NOs: 6-19, biologically active fragments thereof, and/or further variants thereof.

In particular embodiments, an I-OnuI LHE variant that binds and cleaves the human BCL11A gene comprises the following amino acid substitutions: L26V, R28S, R30Q, N32R, N33S, K34D, S35Y, S36A, V37T, S40R, T41I, E42H, G44R, T48G, V68K, A70L, S72V, A76H, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E of I-OnuI (SEQ ID NOs: 1-5) or an I-OnuI variant as set forth in any one of SEQ ID NOs: 6-19, biologically active fragments thereof, and/or further variants thereof.

In particular embodiments, an I-OnuI LHE variant that binds and cleaves the human BCL11A gene comprises the following amino acid substitutions: L26V, R28S, R30Q, N32R, N33S, K34D, S35Y, S36A, V37T, S40R, T41I, E42H, G44R, T48V, V68K, A70S, S72V, A76H, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E of I-OnuI (SEQ ID NOs: 1-5) or an I-OnuI variant as set forth in any one of SEQ ID NOs: 6-19, biologically active fragments thereof, and/or further variants thereof.

In particular embodiments, an I-OnuI LHE variant that binds and cleaves the human BCL11A gene comprises an amino acid sequence that is at least 80%, preferably at least 85%, more preferably at least 90%, or even more preferably at least 95% identical to the amino acid sequence set forth in any one of SEQ ID NOs: 6-19, or a biologically active fragment thereof.

In particular embodiments, an I-OnuI LHE variant comprises an amino acid sequence set forth in any one of SEQ ID NOs: 6-19, or a biologically active fragment thereof.

In particular embodiments, an I-OnuI LHE variant comprises an amino acid sequence set forth in SEQ ID NO: 6, or a biologically active fragment thereof.

In particular embodiments, an I-OnuI LHE variant comprises an amino acid sequence set forth in SEQ ID NO: 7, or a biologically active fragment thereof.

In particular embodiments, an I-OnuI LHE variant comprises an amino acid sequence set forth in SEQ ID NO: 8, or a biologically active fragment thereof.

In particular embodiments, an I-OnuI LHE variant comprises an amino acid sequence set forth in SEQ ID NO: 9, or a biologically active fragment thereof.

In particular embodiments, an I-OnuI LHE variant comprises an amino acid sequence set forth in SEQ ID NO: 10, or a biologically active fragment thereof.

In particular embodiments, an I-OnuI LHE variant comprises an amino acid sequence set forth in SEQ ID NO: 11, or a biologically active fragment thereof.

In particular embodiments, an I-OnuI LHE variant comprises an amino acid sequence set forth in SEQ ID NO: 12, or a biologically active fragment thereof.

In particular embodiments, an I-OnuI LHE variant comprises an amino acid sequence set forth in SEQ ID NO: 13, or a biologically active fragment thereof.

In particular embodiments, an I-OnuI LHE variant comprises an amino acid sequence set forth in SEQ ID NO: 14, or a biologically active fragment thereof.

In particular embodiments, an I-OnuI LHE variant comprises an amino acid sequence set forth in SEQ ID NO: 15, or a biologically active fragment thereof.

In particular embodiments, an I-OnuI LHE variant comprises an amino acid sequence set forth in SEQ ID NO: 16, or a biologically active fragment thereof.

In particular embodiments, an I-OnuI LHE variant comprises an amino acid sequence set forth in SEQ ID NO: 17, or a biologically active fragment thereof.

In particular embodiments, an I-OnuI LHE variant comprises an amino acid sequence set forth in SEQ ID NO: 18, or a biologically active fragment thereof.

In particular embodiments, an I-OnuI LHE variant comprises an amino acid sequence set forth in SEQ ID NO: 19, or a biologically active fragment thereof.

2. MegaTALs

In various embodiments, a megaTAL comprising a homing endonuclease variant is reprogrammed to introduce double-strand breaks (DSBs) in an erythroid specific enhancer in the BCL11A gene, preferably in a GATA-1 binding site in the BCL11A gene, more preferably in a consensus GATA-1 binding site in the second intron of the BCL11A gene, and even more preferably in a target site set forth in SEQ ID NO: 25 (the complement of which includes the Consensus GATA-1 motif WGATAR). A “megaTAL” refers to a polypeptide comprising a TALE DNA binding domain and a homing endonuclease variant that binds and cleaves a DNA target sequence in a BCL11A gene, and optionally comprises one or more linkers and/or additional functional domains, e.g., an end-processing enzymatic domain of an end-processing enzyme that exhibits 5′-3′ exonuclease, 5′-3′ alkaline exonuclease, 3′-5′ exonuclease (e.g., Trex2), 5′ flap endonuclease, helicase or template-independent DNA polymerases activity.

In particular embodiments, a megaTAL can be introduced into a cell along with an end-processing enzyme that exhibits 5′-3′ exonuclease, 5′-3′ alkaline exonuclease, 3′-5′ exonuclease (e.g., Trex2), 5′ flap endonuclease, helicase, template-dependent DNA polymerase or template-independent DNA polymerase activity. The megaTAL and 3′ processing enzyme may be introduced separately, e.g., in different vectors or separate mRNAs, or together, e.g., as a fusion protein, or in a polycistronic construct separated by a viral self-cleaving peptide or an IRES element.

A “TALE DNA binding domain” is the DNA binding portion of transcription activator-like effectors (TALE or TAL-effectors), which mimics plant transcriptional activators to manipulate the plant transcriptome (see e.g., Kay et al., 2007. Science 318:648-651). TALE DNA binding domains contemplated in particular embodiments are engineered de novo or from naturally occurring TALEs, e.g., AvrBs3 from Xanthomonas campestris pv. vesicatoria, Xanthomonas gardneri, Xanthomonas translucens, Xanthomonas axonopodis, Xanthomonas perforans, Xanthomonas alfalfa, Xanthomonas citri, Xanthomonas euvesicatoria, and Xanthomonas oryzae and brg11 and hpx17 from Ralstonia solanacearum. Illustrative examples of TALE proteins for deriving and designing DNA binding domains are disclosed in U.S. Pat. No. 9,017,967, and references cited therein, all of which are incorporated herein by reference in their entireties.

In particular embodiments, a megaTAL comprises a TALE DNA binding domain comprising one or more repeat units that are involved in binding of the TALE DNA binding domain to its corresponding target DNA sequence. A single “repeat unit” (also referred to as a “repeat”) is typically 33-35 amino acids in length. Each TALE DNA binding domain repeat unit includes 1 or 2 DNA-binding residues making up the Repeat Variable Di-Residue (RVD), typically at positions 12 and/or 13 of the repeat. The natural (canonical) code for DNA recognition of these TALE DNA binding domains has been determined such that an HD sequence at positions 12 and 13 leads to a binding to cytosine (C), NG binds to T, NI to A, NN binds to G or A, and NG binds to T. In certain embodiments, non-canonical (atypical) RVDs are contemplated.

Illustrative examples of non-canonical RVDs suitable for use in particular megaTALs contemplated in particular embodiments include, but are not limited to HH, KH, NH, NK, NQ, RH, RN, SS, NN, SN, KN for recognition of guanine (G); NI, KI, RI, HI, SI for recognition of adenine (A); NG, HG, KG, RG for recognition of thymine (T); RD, SD, HD, ND, KD, YG for recognition of cytosine (C); NV, HN for recognition of A or G; and H*, HA, KA, N*, NA, NC, NS, RA, S*for recognition of A or T or G or C, wherein (*) means that the amino acid at position 13 is absent. Additional illustrative examples of RVDs suitable for use in particular megaTALs contemplated in particular embodiments further include those disclosed in U.S. Pat. No. 8,614,092, which is incorporated herein by reference in its entirety.

In particular embodiments, a megaTAL contemplated herein comprises a TALE DNA binding domain comprising 3 to 30 repeat units. In certain embodiments, a megaTAL comprises 3, 4, 5, 6, 7, 8, 9, 10, 11, 12, 13, 14, 15, 16, 17, 18, 19, 20, 21, 22, 23, 24, 25, 26, 27, 28, 29, or 30 TALE DNA binding domain repeat units. In a preferred embodiment, a megaTAL contemplated herein comprises a TALE DNA binding domain comprising 5-15 repeat units, more preferably 7-15 repeat units, more preferably 9-15 repeat units, and more preferably 9, 10, 11, 12, 13, 14, or 15 repeat units.

In particular embodiments, a megaTAL contemplated herein comprises a TALE DNA binding domain comprising 3 to 30 repeat units and an additional single truncated TALE repeat unit comprising 20 amino acids located at the C-terminus of a set of TALE repeat units, i.e., an additional C-terminal half-TALE DNA binding domain repeat unit (amino acids −20 to −1 of the C-cap disclosed elsewhere herein, infra). Thus, in particular embodiments, a megaTAL contemplated herein comprises a TALE DNA binding domain comprising 3.5 to 30.5 repeat units. In certain embodiments, a megaTAL comprises 3.5, 4.5, 5.5, 6.5, 7.5, 8.5, 9.5, 10.5, 11.5, 12.5, 13.5, 14.5, 15.5, 16.5, 17.5, 18.5, 19.5, 20.5, 21.5, 22.5, 23.5, 24.5, 25.5, 26.5, 27.5, 28.5, 29.5, or 30.5 TALE DNA binding domain repeat units. In a preferred embodiment, a megaTAL contemplated herein comprises a TALE DNA binding domain comprising 5.5-15.5 repeat units, more preferably 7.5-15.5 repeat units, more preferably 9.5-15.5 repeat units, and more preferably 9.5, 10.5, 11.5, 12.5, 13.5, 14.5, or 15.5 repeat units.

In particular embodiments, a megaTAL comprises a TAL effector architecture comprising an “N-terminal domain (NTD)” polypeptide, one or more TALE repeat domains/units, a “C-terminal domain (CTD)” polypeptide, and a homing endonuclease variant. In some embodiments, the NTD, TALE repeats, and/or CTD domains are from the same species. In other embodiments, one or more of the NTD, TALE repeats, and/or CTD domains are from different species.

As used herein, the term “N-terminal domain (NTD)” polypeptide refers to the sequence that flanks the N-terminal portion or fragment of a naturally occurring TALE DNA binding domain. The NTD sequence, if present, may be of any length as long as the TALE DNA binding domain repeat units retain the ability to bind DNA. In particular embodiments, the NTD polypeptide comprises at least 120 to at least 140 or more amino acids N-terminal to the TALE DNA binding domain (0 is amino acid 1 of the most N-terminal repeat unit). In particular embodiments, the NTD polypeptide comprises at least about 120, 121, 122, 123, 124, 125, 126, 127, 128, 129, 130, 131, 132, 133, 134, 135, 136, 137, 138, 139, or at least 140 amino acids N-terminal to the TALE DNA binding domain.

In one embodiment, a megaTAL contemplated herein comprises an NTD polypeptide of at least about amino acids +1 to +122 to at least about +1 to +137 of a Xanthomonas TALE protein (0 is amino acid 1 of the most N-terminal repeat unit). In particular embodiments, the NTD polypeptide comprises at least about 122, 123, 124, 125, 126, 127, 128, 129, 130, 131, 132, 133, 134, 135, 136, or 137 amino acids N-terminal to the TALE DNA binding domain of a Xanthomonas TALE protein. In one embodiment, a megaTAL contemplated herein comprises an NTD polypeptide of at least amino acids +1 to +121 of a Ralstonia TALE protein (0 is amino acid 1 of the most N-terminal repeat unit). In particular embodiments, the NTD polypeptide comprises at least about 121, 122, 123, 124, 125, 126, 127, 128, 129, 130, 131, 132, 133, 134, 135, 136, or 137 amino acids N-terminal to the TALE DNA binding domain of a Ralstonia TALE protein.

As used herein, the term “C-terminal domain (CTD)” polypeptide refers to the sequence that flanks the C-terminal portion or fragment of a naturally occurring TALE DNA binding domain. The CTD sequence, if present, may be of any length as long as the TALE DNA binding domain repeat units retain the ability to bind DNA. In particular embodiments, the CTD polypeptide comprises at least 20 to at least 85 or more amino acids C-terminal to the last full repeat of the TALE DNA binding domain (the first 20 amino acids are the half-repeat unit C-terminal to the last C-terminal full repeat unit). In particular embodiments, the CTD polypeptide comprises at least about 20, 21, 22, 23, 24, 25, 26, 27, 28, 29, 30, 31, 32, 33, 34, 35, 36, 37, 38, 39, 40, 41, 42, 443, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or at least 85 amino acids C-terminal to the last full repeat of the TALE DNA binding domain. In one embodiment, a megaTAL contemplated herein comprises a CTD polypeptide of at least about amino acids −20 to −1 of a Xanthomonas TALE protein (−20 is amino acid 1 of a half-repeat unit C-terminal to the last C-terminal full repeat unit). In particular embodiments, the CTD polypeptide comprises at least about 20, 19, 18, 17, 16, 15, 14, 13, 12, 11, 10, 9, 8, 7, 6, 5, 4, 3, 2, or 1 amino acids C-terminal to the last full repeat of the TALE DNA binding domain of a Xanthomonas TALE protein. In one embodiment, a megaTAL contemplated herein comprises a CTD polypeptide of at least about amino acids −20 to −1 of a Ralstonia TALE protein (−20 is amino acid 1 of a half-repeat unit C-terminal to the last C-terminal full repeat unit). In particular embodiments, the CTD polypeptide comprises at least about 20, 19, 18, 17, 16, 15, 14, 13, 12, 11, 10, 9, 8, 7, 6, 5, 4, 3, 2, or 1 amino acids C-terminal to the last full repeat of the TALE DNA binding domain of a Ralstonia TALE protein.

In particular embodiments, a megaTAL contemplated herein, comprises a fusion polypeptide comprising a TALE DNA binding domain engineered to bind a target sequence, a homing endonuclease reprogrammed to bind and cleave a target sequence, and optionally an NTD and/or CTD polypeptide, optionally joined to each other with one or more linker polypeptides contemplated elsewhere herein. Without wishing to be bound by any particular theory, it is contemplated that a megaTAL comprising TALE DNA binding domain, and optionally an NTD and/or CTD polypeptide is fused to a linker polypeptide which is further fused to a homing endonuclease variant. Thus, the TALE DNA binding domain binds a DNA target sequence that is within about 1, 2, 3, 4, 5, 6, 7, 8, 9, 10, 11, 12, 13, 14, or 15 nucleotides away from the target sequence bound by the DNA binding domain of the homing endonuclease variant. In this way, the megaTALs contemplated herein, increase the specificity and efficiency of genome editing.

In one embodiment, a megaTAL comprises a homing endonuclease variant and a TALE DNA binding domain that binds a nucleotide sequence that is within about 4, 5, or 6 nucleotides, preferably, 6 nucleotides upstream of the binding site of the reprogrammed homing endonuclease.

In one embodiment, a megaTAL comprises a homing endonuclease variant and a TALE DNA binding domain that binds the nucleotide sequence set forth in SEQ ID NO: 26, which is 6 nucleotides upstream of the nucleotide sequence bound and cleaved by the homing endonuclease variant (SEQ ID NO: 25). In preferred embodiments, the megaTAL target sequence is SEQ ID NO: 27.

In particular embodiments, a megaTAL contemplated herein, comprises one or more TALE DNA binding repeat units and an LHE variant designed or reprogrammed from an LHE selected from the group consisting of I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, I-Vdil41I and variants thereof, or preferably I-CpaMI, I-HjeMI, I-OnuI, I-PanMI, SmaMI and variants thereof, or more preferably I-OnuI and variants thereof.

In particular embodiments, a megaTAL contemplated herein, comprises an NTD, one or more TALE DNA binding repeat units, a CTD, and an LHE variant selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, I-Vdil41I and variants thereof, or preferably I-CpaMI, I-HjeMI, I-OnuI, I-PanMI, SmaMI and variants thereof, or more preferably I-OnuI and variants thereof.

In particular embodiments, a megaTAL contemplated herein, comprises an NTD, about 9.5 to about 15.5 TALE DNA binding repeat units, and an LHE variant selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, I-Vdil41I and variants thereof, or preferably I-CpaMI, I-HjeMI, I-OnuI, I-PanMI, SmaMI and variants thereof, or more preferably I-OnuI and variants thereof.

In particular embodiments, a megaTAL contemplated herein, comprises an NTD of about 122 amino acids to 137 amino acids, about 9.5, about 10.5, about 11.5, about 12.5, about 13.5, about 14.5, or about 15.5 binding repeat units, a CTD of about 20 amino acids to about 85 amino acids, and an I-OnuI LHE variant. In particular embodiments, any one of, two of, or all of the NTD, DNA binding domain, and CTD can be designed from the same species or different species, in any suitable combination.

In particular embodiments, a megaTAL contemplated herein, comprises the amino acid sequence set forth in any one of SEQ ID NOs: 20 or 21.

In particular embodiments, a megaTAL-Trex2 fusion protein contemplated herein, comprises the amino acid sequence set forth in SEQ ID NO: 22 or 23.

In certain embodiments, a megaTAL comprises a TALE DNA binding domain and an I-OnuI LHE variant binds and cleaves the nucleotide sequence set forth in SEQ ID NO: 27.

3. End-Processing Enzymes

Genome editing compositions and methods contemplated in particular embodiments comprise editing cellular genomes using a nuclease variant and an end-processing enzyme. In particular embodiments, a single polynucleotide encodes a homing endonuclease variant and an end-processing enzyme, separated by a linker, a self-cleaving peptide sequence, e.g., 2A sequence, or by an IRES sequence. In particular embodiments, genome editing compositions comprise a polynucleotide encoding a nuclease variant and a separate polynucleotide encoding an end-processing enzyme.

The term “end-processing enzyme” refers to an enzyme that modifies the exposed ends of a polynucleotide chain. The polynucleotide may be double-stranded DNA (dsDNA), single-stranded DNA (ssDNA), RNA, double-stranded hybrids of DNA and RNA, and synthetic DNA (for example, containing bases other than A, C, G, and T). An end-processing enzyme may modify exposed polynucleotide chain ends by adding one or more nucleotides, removing one or more nucleotides, removing or modifying a phosphate group and/or removing or modifying a hydroxyl group. An end-processing enzyme may modify ends at endonuclease cut sites or at ends generated by other chemical or mechanical means, such as shearing (for example by passing through fine-gauge needle, heating, sonicating, mini bead tumbling, and nebulizing), ionizing radiation, ultraviolet radiation, oxygen radicals, chemical hydrolysis and chemotherapy agents.

In particular embodiments, genome editing compositions and methods contemplated in particular embodiments comprise editing cellular genomes using a homing endonuclease variant or megaTAL and a DNA end-processing enzyme.

The term “DNA end-processing enzyme” refers to an enzyme that modifies the exposed ends of DNA. A DNA end-processing enzyme may modify blunt ends or staggered ends (ends with 5′ or 3′ overhangs). A DNA end-processing enzyme may modify single stranded or double stranded DNA. A DNA end-processing enzyme may modify ends at endonuclease cut sites or at ends generated by other chemical or mechanical means, such as shearing (for example by passing through fine-gauge needle, heating, sonicating, mini bead tumbling, and nebulizing), ionizing radiation, ultraviolet radiation, oxygen radicals, chemical hydrolysis and chemotherapy agents. DNA end-processing enzyme may modify exposed DNA ends by adding one or more nucleotides, removing one or more nucleotides, removing or modifying a phosphate group and/or removing or modifying a hydroxyl group.

Illustrative examples of DNA end-processing enzymes suitable for use in particular embodiments contemplated herein include, but are not limited to: 5′-3′ exonucleases, 5′-3′ alkaline exonucleases, 3′-5′ exonucleases, 5′ flap endonucleases, helicases, phosphatases, hydrolases and template-independent DNA polymerases.

Additional illustrative examples of DNA end-processing enzymes suitable for use in particular embodiments contemplated herein include, but are not limited to, Trex2, Trex1, Trex1 without transmembrane domain, Apollo, Artemis, DNA2, Exol, ExoT, ExoIII, Fen1, Fan1, MreII, Rad2, Rad9, TdT (terminal deoxynucleotidyl transferase), PNKP, RecE, RecJ, RecQ, Lambda exonuclease, Sox, Vaccinia DNA polymerase, exonuclease I, exonuclease III, exonuclease VII, NDK1, NDK5, NDK7, NDK8, WRN, T7-exonuclease Gene 6, avian myeloblastosis virus integration protein (IN), Bloom, Antartic Phophatase, Alkaline Phosphatase, Poly nucleotide Kinase (PNK), ApeI, Mung Bean nuclease, Hex1, TTRAP (TDP2), Sgs1, Sae2, CUP, Pol mu, Pol lambda, MUS81, EME1, EME2, SLX1, SLX4 and UL-12.

In particular embodiments, genome editing compositions and methods for editing cellular genomes contemplated herein comprise polypeptides comprising a homing endonuclease variant or megaTAL and an exonuclease. The term “exonuclease” refers to enzymes that cleave phosphodiester bonds at the end of a polynucleotide chain via a hydrolyzing reaction that breaks phosphodiester bonds at either the 3′ or 5′ end.

Illustrative examples of exonucleases suitable for use in particular embodiments contemplated herein include, but are not limited to: hExoI, Yeast Exol, E. coli Exol, hTREX2, mouse TREX2, rat TREX2, hTREX1, mouse TREX1, rat TREX1, and Rat TREX1.

In particular embodiments, the DNA end-processing enzyme is a 3′ or 5′ exonuclease, preferably Trex 1 or Trex2, more preferably Trex2, and even more preferably human or mouse Trex2.

### D. Target Sites

Nuclease variants contemplated in particular embodiments can be designed to bind to any suitable target sequence and can have a novel binding specificity, compared to a naturally-occurring nuclease. In particular embodiments, the target site is a regulatory region of a gene including, but not limited to promoters, enhancers, repressor elements, and the like. In particular embodiments, the target site is a coding region of a gene or a splice site. In certain embodiments, nuclease variants are designed to down-regulate or decrease expression of a gene. In particular embodiments, a nuclease variant and donor repair template can be designed to delete a desired target sequence.

In various embodiments, nuclease variants bind to and cleave a target sequence in the B Cell CLL/Lymphoma 11A (BCL11A) gene. The BCL11A gene encodes a C2H2 type zinc-finger transcription factor similar to the mouse Bcl11a/Evi9 protein. BCL11A is a transcriptional repressor that plays a role in the regulation of globin gene expression. In fetal development, full-length forms of BCL11A are not expressed and erythroid cells produce γ-globin which complexes with α-globin to form fetal hemoglobin (HbF). Around birth, BCL11A expression increases in erythroid cells, binds to transcriptional elements in the γ-globin promoter and suppresses or represses γ-globin expression, which is associated with increased β-globin expression. The increase in β-globin expression at the expense of γ-globin leads to a “globin switch” from HbF to HbA (two β-globins/two α-globins). However, in subjects having one or more mutations in the β-globin gene that result in a hemoglobinopathy, switching γ-globin gene expression back on and at the expense of mutated β-globin gene expression would potentially treat the hemoglobinopathy. One solution is to decrease BCL11A expression to derepress γ-globin gene expression and decrease mutated β-globin gene expression.

In particular embodiments, a homing endonuclease variant or megaTAL introduces a double-strand break (DSB) in an erythroid specific enhancer in the BCL11A gene, preferably in a GATA-1 binding site in the BCL11A gene, more preferably in a consensus GATA-1 binding site in the second intron of the BCL11A gene, and even more preferably in a target site set forth in SEQ ID NO: 25 (the complement of which includes the Consensus GATA-1 motif WGATAR). In particular embodiments, the reprogrammed nuclease or megaTAL comprises an I-OnuI LHE variant that introduces a double strand break at the GATA-1 site in the second intron of the BCL11A gene by cleaving the sequence “TTAT” on the strand complementary to the consensus GATA-1 binding motif (WGATAA).

In a preferred embodiment, a homing endonuclease variant or megaTAL is cleaves double-stranded DNA and introduces a DSB into the polynucleotide sequence set forth in SEQ ID NO: 25 or 27.

In a preferred embodiment, the BCL11A gene is a human BCL11A gene.

### E. Donor Repair Templates

Nuclease variants may be used to introduce a DSB in a target sequence; the DSB may be repaired through homology directed repair (HDR) mechanisms in the presence of one or more donor repair templates. In particular embodiments, the donor repair template is used to insert a sequence into the genome. In particular preferred embodiments, the donor repair template is used to delete or repair a genomic sequence in the genome.

In various embodiments, a donor repair template is introduced into a hematopoietic cell, e.g., a hematopoietic stem or progenitor cell, or CD34⁺ cell, by transducing the cell with an adeno-associated virus (AAV), retrovirus, e.g., lentivirus, IDLV, etc., herpes simplex virus, adenovirus, or vaccinia virus vector comprising the donor repair template.

In particular embodiments, the donor repair template comprises one or more homology arms that flank the DSB site.

As used herein, the term “homology arms” refers to a nucleic acid sequence in a donor repair template that is identical, or nearly identical, to DNA sequence flanking the DNA break introduced by the nuclease at a target site. In one embodiment, the donor repair template comprises a 5′ homology arm that comprises a nucleic acid sequence that is identical or nearly identical to the DNA sequence 5′ of the DNA break site. In one embodiment, the donor repair template comprises a 3′ homology arm that comprises a nucleic acid sequence that is identical or nearly identical to the DNA sequence 3′ of the DNA break site. In a preferred embodiment, the donor repair template comprises a 5′ homology arm and a 3′ homology arm. The donor repair template may comprise homology to the genome sequence immediately adjacent to the DSB site, or homology to the genomic sequence within any number of base pairs from the DSB site. In one embodiment, the donor repair template comprises a nucleic acid sequence that is homologous to a genomic sequence about 5 bp, about 10 bp, about 25 bp, about 50 bp, about 100 bp, about 250 bp, about 500 bp, about 1000 bp, about 2500 bp, about 5000 bp, about 10000 bp or more, including any intervening length of homologous sequence.

Illustrative examples of suitable lengths of homology arms contemplated in particular embodiments, may be independently selected, and include but are not limited to: about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp, about 1500 bp, about 1600 bp, about 1700 bp, about 1800 bp, about 1900 bp, about 2000 bp, about 2100 bp, about 2200 bp, about 2300 bp, about 2400 bp, about 2500 bp, about 2600 bp, about 2700 bp, about 2800 bp, about 2900 bp, or about 3000 bp, or longer homology arms, including all intervening lengths of homology arms.

Additional illustrative examples of suitable homology arm lengths include, but are not limited to: about 100 bp to about 3000 bp, about 200 bp to about 3000 bp, about 300 bp to about 3000 bp, about 400 bp to about 3000 bp, about 500 bp to about 3000 bp, about 500 bp to about 2500 bp, about 500 bp to about 2000 bp, about 750 bp to about 2000 bp, about 750 bp to about 1500 bp, or about 1000 bp to about 1500 bp, including all intervening lengths of homology arms.

In a particular embodiment, the lengths of the 5′ and 3′ homology arms are independently selected from about 500 bp to about 1500 bp. In one embodiment, the 5′-homology arm is about 1500 bp and the 3′ homology arm is about 1000 bp. In one embodiment, the 5′-homology arm is between about 200 bp to about 600 bp and the 3′ homology arm is between about 200 bp to about 600 bp. In one embodiment, the 5′-homology arm is about 200 bp and the 3′ homology arm is about 200 bp. In one embodiment, the 5′-homology arm is about 300 bp and the 3′ homology arm is about 300 bp. In one embodiment, the 5′-homology arm is about 400 bp and the 3′ homology arm is about 400 bp. In one embodiment, the 5′-homology arm is about 500 bp and the 3′ homology arm is about 500 bp. In one embodiment, the 5′-homology arm is about 600 bp and the 3′ homology arm is about 600 bp.

### F. Polypeptides

Various polypeptides are contemplated herein, including, but not limited to, homing endonuclease variants, megaTALs, and fusion polypeptides. In preferred embodiments, a polypeptide comprises the amino acid sequence set forth in SEQ ID NOs: 1-23 and 39. “Polypeptide,” “polypeptide fragment,” “peptide” and “protein” are used interchangeably, unless specified to the contrary, and according to conventional meaning, i.e., as a sequence of amino acids. In one embodiment, a “polypeptide” includes fusion polypeptides and other variants. Polypeptides can be prepared using any of a variety of well-known recombinant and/or synthetic techniques. Polypeptides are not limited to a specific length, e.g., they may comprise a full length protein sequence, a fragment of a full length protein, or a fusion protein, and may include post-translational modifications of the polypeptide, for example, glycosylations, acetylations, phosphorylations and the like, as well as other modifications known in the art, both naturally occurring and non-naturally occurring.

An “isolated protein,” “isolated peptide,” or “isolated polypeptide” and the like, as used herein, refer to in vitro synthesis, isolation, and/or purification of a peptide or polypeptide molecule from a cellular environment, and from association with other components of the cell, i.e., it is not significantly associated with in vivo substances.

Illustrative examples of polypeptides contemplated in particular embodiments include, but are not limited to homing endonuclease variants, megaTALs, end-processing nucleases, fusion polypeptides and variants thereof.

Polypeptides include “polypeptide variants.” Polypeptide variants may differ from a naturally occurring polypeptide in one or more amino acid substitutions, deletions, additions and/or insertions. Such variants may be naturally occurring or may be synthetically generated, for example, by modifying one or more amino acids of the above polypeptide sequences. For example, in particular embodiments, it may be desirable to improve the biological properties of a homing endonuclease, megaTAL or the like that binds and cleaves a target site in the human BCL11A gene by introducing one or more substitutions, deletions, additions and/or insertions into the polypeptide. In particular embodiments, polypeptides include polypeptides having at least about 65%, 70%, 71%, 72%, 73%, 74%, 75% 75%, 76%, 77%, 78%, 79%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity to any of the reference sequences contemplated herein, typically where the variant maintains at least one biological activity of the reference sequence.

Polypeptides variants include biologically active “polypeptide fragments.” Illustrative examples of biologically active polypeptide fragments include DNA binding domains, nuclease domains, and the like. As used herein, the term “biologically active fragment” or “minimal biologically active fragment” refers to a polypeptide fragment that retains at least 100%, at least 90%, at least 80%, at least 70%, at least 60%, at least 50%, at least 40%, at least 30%, at least 20%, at least 10%, or at least 5% of the naturally occurring polypeptide activity. In preferred embodiments, the biological activity is binding affinity and/or cleavage activity for a target sequence. In certain embodiments, a polypeptide fragment can comprise an amino acid chain at least 5 to about 1700 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, 450, 500, 550, 600, 650, 700, 750, 800, 850, 900, 950, 1000, 1100, 1200, 1300, 1400, 1500, 1600, 1700 or more amino acids long. In particular embodiments, a polypeptide comprises a biologically active fragment of a homing endonuclease variant. In particular embodiments, the polypeptides set forth herein may comprise one or more amino acids denoted as “X.” “X” if present in an amino acid SEQ ID NO, refers to any amino acid. One or more “X” residues may be present at the N- and C-terminus of an amino acid sequence set forth in particular SEQ ID NOs contemplated herein. If the “X” amino acids are not present the remaining amino acid sequence set forth in a SEQ ID NO may be considered a biologically active fragment.

In particular embodiments, a polypeptide comprises a biologically active fragment of a homing endonuclease variant, e.g., SEQ ID NOs: 3-19 or a megaTAL (SEQ ID NOs: 20-21). The biologically active fragment may comprise an N-terminal truncation and/or C-terminal truncation. In a particular embodiment, a biologically active fragment lacks or comprises a deletion of the 1, 2, 3, 4, 5, 6, 7, or 8 N-terminal amino acids of a homing endonuclease variant compared to a corresponding wild type homing endonuclease sequence, more preferably a deletion of the 4 N-terminal amino acids of a homing endonuclease variant compared to a corresponding wild type homing endonuclease sequence. In a particular embodiment, a biologically active fragment lacks or comprises a deletion of the 1, 2, 3, 4, or 5 C-terminal amino acids of a homing endonuclease variant compared to a corresponding wild type homing endonuclease sequence, more preferably a deletion of the 2 C-terminal amino acids of a homing endonuclease variant compared to a corresponding wild type homing endonuclease sequence. In a particular preferred embodiment, a biologically active fragment lacks or comprises a deletion of the 4 N-terminal amino acids and 2 C-terminal amino acids of a homing endonuclease variant compared to a corresponding wild type homing endonuclease sequence.

In a particular embodiment, an I-OnuI variant comprises a deletion of 1, 2, 3, 4, 5, 6, 7, or 8 the following N-terminal amino acids: M, A, Y, M, S, R, R, E; and/or a deletion of the following 1, 2, 3, 4, or 5 C-terminal amino acids: R, G, S, F, V.

In a particular embodiment, an I-OnuI variant comprises a deletion or substitution of 1, 2, 3, 4, 5, 6, 7, or 8 the following N-terminal amino acids: M, A, Y, M, S, R, R, E; and/or a deletion or substitution of the following 1, 2, 3, 4, or 5 C-terminal amino acids: R, G, S, F, V.

In a particular embodiment, an I-OnuI variant comprises a deletion of 1, 2, 3, 4, 5, 6, 7, or 8 the following N-terminal amino acids: M, A, Y, M, S, R, R, E; and/or a deletion of the following 1 or 2 C-terminal amino acids: F, V.

In a particular embodiment, an I-OnuI variant comprises a deletion or substitution of 1, 2, 3, 4, 5, 6, 7, or 8 the following N-terminal amino acids: M, A, Y, M, S, R, R, E; and/or a deletion or substitution of the following 1 or 2 C-terminal amino acids: F, V.

As noted above, polypeptides may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

In certain embodiments, a variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides contemplated in particular embodiments, polypeptides include polypeptides having at least about and still obtain a functional molecule that encodes a variant or derivative polypeptide with desirable characteristics. When it is desired to alter the amino acid sequence of a polypeptide to create an equivalent, or even an improved, variant polypeptide, one skilled in the art, for example, can change one or more of the codons of the encoding DNA sequence, e.g., according to Table 1.

TABLE 1
Amino Acid Codons

| | One | Three | | | | | | |
|---|---|---|---|---|---|---|---|---|
| | letter | letter | | | | | | |
| Amino Acids | code | code | Codons | | | | | |
| Alanine | A | Ala | GCA | GCC | GCG | GCU | | |
| Cysteine | C | Cys | UGC | UGU | | | | |
| Aspartic acid | D | Asp | GAC | GAU | | | | |
| Glutamic acid | E | Glu | GAA | GAG | | | | |
| Phenylalanine | F | Phe | UUC | UUU | | | | |
| Glycine | G | Gly | GGA | GGC | GGG | GGU | | |
| Histidine | H | His | CAC | CAU | | | | |
| Isoleucine | I | Iso | AUA | AUC | AUU | | | |
| Lysine | K | Lys | AAA | AAG | | | | |
| Leucine | L | Leu | UUA | UUG | CUA | CUC | CUG | CUU |
| Methionine | M | Met | AUG | | | | | |
| Asparagine | N | Asn | AAC | AAU | | | | |
| Proline | P | Pro | CCA | CCC | CCG | CCU | | |
| Glutamine | Q | Gln | CAA | CAG | | | | |
| Arginine | R | Arg | AGA | AGG | CGA | CGC | CGG | CGU |
| Serine | S | Ser | AGC | AGU | UCA | UCC | UCG | UCU |
| Threonine | T | Thr | ACA | ACC | ACG | ACU | | |
| Valine | V | Val | GUA | GUC | GUG | GUU | | |
| Tryptophan | W | Trp | UGG | | | | | |
| Tyrosine | Y | Tyr | UAC | UAU | | | | |

Guidance in determining which amino acid residues can be substituted, inserted, or deleted without abolishing biological activity can be found using computer programs well known in the art, such as DNASTAR, DNA Strider, Geneious, Mac Vector, or Vector NTI software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224).

In one embodiment, where expression of two or more polypeptides is desired, the polynucleotide sequences encoding them can be separated by and IRES sequence as disclosed elsewhere herein.

Polypeptides contemplated in particular embodiments include fusion polypeptides. In particular embodiments, fusion polypeptides and polynucleotides encoding fusion polypeptides are provided. Fusion polypeptides and fusion proteins refer to a polypeptide having at least two, three, four, five, six, seven, eight, nine, or ten polypeptide segments.

In another embodiment, two or more polypeptides can be expressed as a fusion protein that comprises one or more self-cleaving polypeptide sequences as disclosed elsewhere herein.

In one embodiment, a fusion protein contemplated herein comprises one or more DNA binding domains and one or more nucleases, and one or more linker and/or self-cleaving polypeptides.

In one embodiment, a fusion protein contemplated herein comprises a nuclease variant; a linker or self-cleaving peptide; and an end-processing enzyme including but not limited to a 5′-3′ exonuclease, a 5′-3′ alkaline exonuclease, and a 3′-5′ exonuclease (e.g., Trex2).

Fusion polypeptides can comprise one or more polypeptide domains or segments including, but are not limited to signal peptides, cell permeable peptide domains (CPP), DNA binding domains, nuclease domains, etc., epitope tags (e.g., maltose binding protein (“MBP”), glutathione S transferase (GST), HIS6, MYC, FLAG, V5, VSV-G, and HA), polypeptide linkers, and polypeptide cleavage signals. Fusion polypeptides are typically linked C-terminus to N-terminus, although they can also be linked C-terminus to C-terminus, N-terminus to N-terminus, or N-terminus to C-terminus. In particular embodiments, the polypeptides of the fusion protein can be in any order. Fusion polypeptides or fusion proteins can also include conservatively modified variants, polymorphic variants, alleles, mutants, subsequences, and interspecies homologs, so long as the desired activity of the fusion polypeptide is preserved. Fusion polypeptides may be produced by chemical synthetic methods or by chemical linkage between the two moieties or may generally be prepared using other standard techniques. Ligated DNA sequences comprising the fusion polypeptide are operably linked to suitable transcriptional or translational control elements as disclosed elsewhere herein.

Fusion polypeptides may optionally comprise a linker that can be used to link the one or more polypeptides or domains within a polypeptide. A peptide linker sequence may be employed to separate any two or more polypeptide components by a distance sufficient to ensure that each polypeptide folds into its appropriate secondary and tertiary structures so as to allow the polypeptide domains to exert their desired functions. Such a peptide linker sequence is incorporated into the fusion polypeptide using standard techniques in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. Nos. 4,935,233 and 4,751,180. Linker sequences are not required when a particular fusion polypeptide segment contains non-essential N-terminal amino acid regions that can be used to separate the functional domains and prevent steric interference. Preferred linkers are typically flexible amino acid subsequences which are synthesized as part of a recombinant fusion protein. Linker polypeptides can be between 1 and 200 amino acids in length, between 1 and 100 amino acids in length, or between 1 and 50 amino acids in length, including all integer values in between.

Exemplary linkers include, but are not limited to the following amino acid sequences: glycine polymers (G)ₙ; glycine-serine polymers (G₁₋₅S₁₋₅)ₙ, where n is an integer of at least one, two, three, four, or five; glycine-alanine polymers; alanine-serine polymers; GGG (SEQ ID NO: 40); DGGGS (SEQ ID NO: 41); TGEKP (SEQ ID NO: 42) (see e.g., Liu et al., PNAS 5525-5530 (1997)); GGRR (SEQ ID NO: 43) (Pomerantz et al. 1995, supra); (GGGGS)ₙ wherein n=1, 2, 3, 4 or 5 (SEQ ID NO: 44) (Kim et al., PNAS 93, 1156-1160 (1996.); EGKSSGSGSESKVD (SEQ ID NO: 45) (Chaudhary et al., 1990, Proc. Natl. Acad. Sci. U.S.A. 87:1066-1070); KESGSVSSEQLAQFRSLD (SEQ ID NO 46) (Bird et al., 1988, Science 242:423-426), GGRRGGGS (SEQ ID NO: 47); LRQRDGERP (SEQ ID NO: 48); LRQKDGGGSERP (SEQ ID NO: 49); LRQKD(GGGS)₂ERP (SEQ ID NO: 50). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods.

Fusion polypeptides may further comprise a polypeptide cleavage signal between each of the polypeptide domains described herein or between an endogenous open reading frame and a polypeptide encoded by a donor repair template. In addition, a polypeptide cleavage site can be put into any linker peptide sequence. Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5(8); 616-26).

Suitable protease cleavages sites and self-cleaving peptides are known to the skilled person (see, e.g., in Ryan et al., 1997. J Gener. Virol. 78, 699-722; Scymczak et al. (2004) Nature Biotech. 5, 589-594). Exemplary protease cleavage sites include, but are not limited to the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picoma 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are preferred in one embodiment, e.g., EXXYXQ(G/S) (SEQ ID NO: 51), for example, ENLYFQG (SEQ ID NO: 52) and ENLYFQS (SEQ ID NO: 53), wherein X represents any amino acid (cleavage by TEV occurs between Q and G or Q and S).

In certain embodiments, the self-cleaving polypeptide site comprises a 2A or 2A-like site, sequence or domain (Donnelly et al., 2001. J. Gen. Virol. 82:1027-1041). In a particular embodiment, the viral 2A peptide is an aphthovirus 2A peptide, a potyvirus 2A peptide, or a cardiovirus 2A peptide.

In one embodiment, the viral 2A peptide is selected from the group consisting of: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

Illustrative examples of 2A sites are provided in Table 2.

TABLE 2
Exemplary 2A sites include the following sequences:

| | |
|---|---|
| SEQ ID NO: 54 | GSGATNFSLLKQAGDVEENPGP |
| SEQ ID NO: 55 | ATNFSLLKQAGDVEENPGP |
| SEQ ID NO: 56 | LLKQAGDVEENPGP |
| SEQ ID NO: 57 | GSGEGRGSLLTCGDVEENPGP |
| SEQ ID NO: 58 | EGRGSLLTCGDVEENPGP |
| SEQ ID NO: 59 | LLTCGDVEENPGP |
| SEQ ID NO: 60 | GSGQCTNYALLKLAGDVESNPGP |
| SEQ ID NO: 61 | QCTNYALLKLAGDVESNPGP |
| SEQ ID NO: 62 | LLKLAGDVESNPGP |
| SEQ ID NO: 63 | GSGVKQTLNFDLLKLAGDVESNPGP |
| SEQ ID NO: 64 | VKQTLNFDLLKLAGDVESNPGP |
| SEQ ID NO: 65 | LLKLAGDVESNPGP |
| SEQ ID NO: 66 | LLNFDLLKLAGDVESNPGP |
| SEQ ID NO: 67 | TLNFDLLKLAGDVESNPGP |
| SEQ ID NO: 68 | LLKLAGDVESNPGP |
| SEQ ID NO: 69 | NFDLLKLAGDVESNPGP |
| SEQ ID NO: 70 | QLLNFDLLKLAGDVESNPGP |
| SEQ ID NO: 71 | APVKQTLNFDLLKLAGDVESNPGP |
| SEQ ID NO: 72 | VTELLYRMKRAETYCPRPLLAIHPTEARHKQKIVAPVKQT |
| SEQ ID NO: 73 | LNFDLLKLAGDVESNPGP |
| SEQ ID NO: 74 | LLAIHPTEARHKQKIVAPVKQTLNFDLLKLAGDVESNPGP |
| SEQ ID NO: 75 | EARHKQKIVAPVKQTLNFDLLKLAGDVESNPGP |

### G. Polynucleotides

In particular embodiments, polynucleotides encoding one or more homing endonuclease variants, megaTALs, end-processing enzymes, and fusion polypeptides contemplated herein are provided. As used herein, the terms “polynucleotide” or “nucleic acid” refer to deoxyribonucleic acid (DNA), ribonucleic acid (RNA) and DNA/RNA hybrids. Polynucleotides may be single-stranded or double-stranded and either recombinant, synthetic, or isolated. Polynucleotides include, but are not limited to: pre-messenger RNA (pre-mRNA), messenger RNA (mRNA), RNA, short interfering RNA (siRNA), short hairpin RNA (shRNA), microRNA (miRNA), ribozymes, genomic RNA (gRNA), plus strand RNA (RNA(+)), minus strand RNA (RNA(−)), tracrRNA, crRNA, single guide RNA (sgRNA), synthetic RNA, synthetic mRNA, genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxyribonucleotides or a modified form of either type of nucleotide, as well as all intermediate lengths. It will be readily understood that “intermediate lengths, “in this context, means any length between the quoted values, such as 6, 7, 8, 9, etc., 101, 102, 103, etc.; 151, 152, 153, etc.; 201, 202, 203, etc. In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence.

In particular embodiments, polynucleotides may be codon-optimized. As used herein, the term “codon-optimized” refers to substituting codons in a polynucleotide encoding a polypeptide in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, and/or (x) systematic variation of codon sets for each amino acid, and/or (xi) isolated removal of spurious translation initiation sites.

As used herein the term “nucleotide” refers to a heterocyclic nitrogenous base in N-glycosidic linkage with a phosphorylated sugar. Nucleotides are understood to include natural bases, and a wide variety of art-recognized modified bases. Such bases are generally located at the 1′ position of a nucleotide sugar moiety. Nucleotides generally comprise a base, sugar and a phosphate group. In ribonucleic acid (RNA), the sugar is a ribose, and in deoxyribonucleic acid (DNA) the sugar is a deoxyribose, i.e., a sugar lacking a hydroxyl group that is present in ribose. Exemplary natural nitrogenous bases include the purines, adenosine (A) and guanidine (G), and the pyrimidines, cytidine (C) and thymidine (T) (or in the context of RNA, uracil (U)). The C-1 atom of deoxyribose is bonded to N-1 of a pyrimidine or N-9 of a purine. Nucleotides are usually mono, di- or triphosphates. The nucleotides can be unmodified or modified at the sugar, phosphate and/or base moiety, (also referred to interchangeably as nucleotide analogs, nucleotide derivatives, modified nucleotides, non-natural nucleotides, and non-standard nucleotides; see for example, WO 92/07065 and WO 93/15187). Examples of modified nucleic acid bases are summarized by Limbach et al., (1994, Nucleic Acids Res. 22, 2183-2196).

A nucleotide may also be regarded as a phosphate ester of a nucleoside, with esterification occurring on the hydroxyl group attached to C-5 of the sugar. As used herein, the term “nucleoside” refers to a heterocyclic nitrogenous base in N-glycosidic linkage with a sugar. Nucleosides are recognized in the art to include natural bases, and also to include well known modified bases. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, nucleoside derivatives, modified nucleosides, non-natural nucleosides, or non-standard nucleosides). As also noted above, examples of modified nucleic acid bases are summarized by Limbach et al., (1994, Nucleic Acids Res. 22, 2183-2196).

Illustrative examples of polynucleotides include, but are not limited to polynucleotides encoding SEQ ID NOs: 1-19 and 39 and polynucleotide sequences set forth in SEQ ID NOs: 20-38.

In various illustrative embodiments, polynucleotides contemplated herein include, but are not limited to polynucleotides encoding homing endonuclease variants, megaTALs, end-processing enzymes, fusion polypeptides, and expression vectors, viral vectors, and transfer plasmids comprising polynucleotides contemplated herein.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion, substitution, or modification of at least one nucleotide. Accordingly, the terms “polynucleotide variant” and “variant” include polynucleotides in which one or more nucleotides have been added or deleted, or modified, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide.

In one embodiment, a polynucleotide comprises a nucleotide sequence that hybridizes to a target nucleic acid sequence under stringent conditions. To hybridize under “stringent conditions” describes hybridization protocols in which nucleotide sequences at least 60% identical to each other remain hybridized. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 96%, 97%, 98%, 99% or 100% sequence identity to any of the reference sequences described herein, typically where the polypeptide variant maintains at least one biological activity of the reference polypeptide.

Terms used to describe sequence relationships between two or more polynucleotides or polypeptides include “reference sequence,” “comparison window,” “sequence identity,” “percentage of sequence identity,” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc., 1994-1998, Chapter 15.

An “isolated polynucleotide,” as used herein, refers to a polynucleotide that has been purified from the sequences which flank it in a naturally-occurring state, e.g., a DNA fragment that has been removed from the sequences that are normally adjacent to the fragment. In particular embodiments, an “isolated polynucleotide” refers to a complementary DNA (cDNA), a recombinant polynucleotide, a synthetic polynucleotide, or other polynucleotide that does not exist in nature and that has been made by the hand of man.

In various embodiments, a polynucleotide comprises an mRNA encoding a polypeptide contemplated herein including, but not limited to, a homing endonuclease variant, a megaTAL, and an end-processing enzyme. In certain embodiments, the mRNA comprises a cap, one or more nucleotides, and a poly(A) tail.

As used herein, the terms “5′ cap” or “5′ cap structure” or “5′ cap moiety” refer to a chemical modification, which has been incorporated at the 5′ end of an mRNA. The 5′ cap is involved in nuclear export, mRNA stability, and translation.

In particular embodiments, a mRNA contemplated herein comprises a 5′ cap comprising a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue.

Illustrative examples of 5′ cap suitable for use in particular embodiments of the mRNA polynucleotides contemplated herein include, but are not limited to: unmethylated 5′ cap analogs, e.g., G(5′)ppp(5′)G, G(5′)ppp(5′)C, G(5′)ppp(5′)A; methylated 5′ cap analogs, e.g., m⁷G(5′)ppp(5′)G, m⁷G(5′)ppp(5′)C, and m⁷G(5′)ppp(5′)A; dimethylated 5′ cap analogs, e.g., m^{2,7}G(5′)ppp(5′)G, m^{2,7}G(5′)ppp(5′)C, and m^{2,7}G(5′)ppp(5′)A; trimethylated 5′ cap analogs, e.g., m^{2,2,7}G(5′)ppp(5′)G, m^{2,2,7}G(5′)ppp(5′)C, and m^{2,2,7}G(5′)ppp(5′)A; dimethylated symmetrical 5′ cap analogs, e.g., m⁷G(5′)pppm⁷(5′)G, m⁷G(5′)pppm⁷(5′)C, and m⁷G(5′)pppm⁷(5′)A; and anti-reverse 5′ cap analogs, e.g., Anti-Reverse Cap Analog (ARCA) cap, designated 3′O-Me-m⁷G(5′)ppp(5′)G, 2′O-Me-m⁷G(5′)ppp(5′)G, 2′O-Me-m⁷G(5′)ppp(5′)C, 2′O-Me-m⁷G(5′)ppp(5′)A, m⁷2′d(5′)ppp(5′)G, m⁷2′d(5′)ppp(5′)C, m⁷2′d(5′)ppp(5′)A, 3′O-Me-m⁷G(5′)ppp(5′)C, 3′O-Me-m⁷G(5′)ppp(5′)A, m⁷3′d(5′)ppp(5′)G, m⁷3′d(5′)ppp(5′)C, m⁷3′d(5′)ppp(5′)A and their tetraphosphate derivatives) (see, e.g., Jemielity et al., RNA, 9: 1108-1122 (2003)).

In particular embodiments, mRNAs comprise a 5′ cap that is a 7-methyl guanylate (“m⁷G”) linked via a triphosphate bridge to the 5′-end of the first transcribed nucleotide, resulting in m⁷G(5′)ppp(5′)N, where N is any nucleoside.

In some embodiments, mRNAs comprise a 5′ cap wherein the cap is a Cap0 structure (Cap0 structures lack a 2′-O-methyl residue of the ribose attached to bases 1 and 2), a Cap1 structure (Cap1 structures have a 2′-O-methyl residue at base 2), or a Cap2 structure (Cap2 structures have a 2′-O-methyl residue attached to both bases 2 and 3).

In one embodiment, an mRNA comprises an m⁷G(5′)ppp(5′)G cap.

In one embodiment, an mRNA comprises an ARCA cap.

In particular embodiments, an mRNA contemplated herein comprises one or more modified nucleosides.

In one embodiment, an mRNA comprises one or more modified nucleosides selected from the group consisting of: pseudouridine, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In one embodiment, an mRNA comprises one or more modified nucleosides selected from the group consisting of: pseudouridine, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In one embodiment, an mRNA comprises one or more modified nucleosides selected from the group consisting of: 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In one embodiment, an mRNA comprises one or more modified nucleosides selected from the group consisting of: 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In one embodiment, an mRNA comprises one or more modified nucleosides selected from the group consisting of: inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In one embodiment, an mRNA comprises one or more pseudouridines, one or more 5-methyl-cytosines, and/or one or more 5-methyl-cytidines.

In one embodiment, an mRNA comprises one or more pseudouridines.

In one embodiment, an mRNA comprises one or more 5-methyl-cytidines.

In one embodiment, an mRNA comprises one or more 5-methyl-cytosines.

In particular embodiments, an mRNA contemplated herein comprises a poly(A) tail to help protect the mRNA from exonuclease degradation, stabilize the mRNA, and facilitate translation. In certain embodiments, an mRNA comprises a 3′ poly(A) tail structure.

In particular embodiments, the length of the poly(A) tail is at least about 10, 25, 50, 75, 100, 150, 200, 250, 300, 350, 400, 450, or at least about 500 or more adenine nucleotides or any intervening number of adenine nucleotides. In particular embodiments, the length of the poly(A) tail is at least about 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 202, 203, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, or 275 or more adenine nucleotides.

In particular embodiments, the length of the poly(A) tail is about 10 to about 500 adenine nucleotides, about 50 to about 500 adenine nucleotides, about 100 to about 500 adenine nucleotides, about 150 to about 500 adenine nucleotides, about 200 to about 500 adenine nucleotides, about 250 to about 500 adenine nucleotides, about 300 to about 500 adenine nucleotides, about 50 to about 450 adenine nucleotides, about 50 to about 400 adenine nucleotides, about 50 to about 350 adenine nucleotides, about 100 to about 500 adenine nucleotides, about 100 to about 450 adenine nucleotides, about 100 to about 400 adenine nucleotides, about 100 to about 350 adenine nucleotides, about 100 to about 300 adenine nucleotides, about 150 to about 500 adenine nucleotides, about 150 to about 450 adenine nucleotides, about 150 to about 400 adenine nucleotides, about 150 to about 350 adenine nucleotides, about 150 to about 300 adenine nucleotides, about 150 to about 250 adenine nucleotides, about 150 to about 200 adenine nucleotides, about 200 to about 500 adenine nucleotides, about 200 to about 450 adenine nucleotides, about 200 to about 400 adenine nucleotides, about 200 to about 350 adenine nucleotides, about 200 to about 300 adenine nucleotides, about 250 to about 500 adenine nucleotides, about 250 to about 450 adenine nucleotides, about 250 to about 400 adenine nucleotides, about 250 to about 350 adenine nucleotides, or about 250 to about 300 adenine nucleotides or any intervening range of adenine nucleotides.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the pre-messenger (pre-mRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

The terms “complementary” and “complementarity” refer to polynucleotides (i.e., a sequence of nucleotides) related by the base-pairing rules. For example, the complementary strand of the DNA sequence 5′ A G T C A T G 3′ is 3′ T C A G T A C 5′. The latter sequence is often written as the reverse complement with the 5′ end on the left and the 3′ end on the right, 5′ C A T G A C T 3′. A sequence that is equal to its reverse complement is said to be a palindromic sequence. Complementarity can be “partial,” in which only some of the nucleic acids' bases are matched according to the base pairing rules. Or, there can be “complete” or “total” complementarity between the nucleic acids.

The term “nucleic acid cassette” or “expression cassette” as used herein refers to genetic sequences within the vector which can express an RNA, and subsequently a polypeptide. In one embodiment, the nucleic acid cassette contains a gene(s)-of-interest, e.g., a polynucleotide(s)-of-interest. In another embodiment, the nucleic acid cassette contains one or more expression control sequences, e.g., a promoter, enhancer, poly(A) sequence, and a gene(s)-of-interest, e.g., a polynucleotide(s)-of-interest. Vectors may comprise 1, 2, 3, 4, 5, 6, 7, 8, 9 or 10 or more nucleic acid cassettes. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. In a preferred embodiment, the nucleic acid cassette contains the sequence of a therapeutic gene used to treat, prevent, or ameliorate a genetic disorder. The cassette can be removed and inserted into a plasmid or viral vector as a single unit.

Polynucleotides include polynucleotide(s)-of-interest. As used herein, the term “polynucleotide-of-interest” refers to a polynucleotide encoding a polypeptide or fusion polypeptide or a polynucleotide that serves as a template for the transcription of an inhibitory polynucleotide, as contemplated herein.

Moreover, it will be appreciated by those of ordinary skill in the art that, as a result of the degeneracy of the genetic code, there are many nucleotide sequences that may encode a polypeptide, or fragment of variant thereof, as contemplated herein. Some of these polynucleotides bear minimal homology to the nucleotide sequence of any native gene. Nonetheless, polynucleotides that vary due to differences in codon usage are specifically contemplated in particular embodiments, for example polynucleotides that are optimized for human and/or primate codon selection. In one embodiment, polynucleotides comprising particular allelic sequences are provided. Alleles are endogenous polynucleotide sequences that are altered as a result of one or more mutations, such as deletions, additions and/or substitutions of nucleotides.

In a certain embodiment, a polynucleotide-of-interest comprises a donor repair template.

In a certain embodiment, a polynucleotide-of-interest comprises an inhibitory polynucleotide including, but not limited to, an siRNA, an miRNA, an shRNA, a ribozyme or another inhibitory RNA.

In one embodiment, a donor repair template comprising an inhibitory RNA comprises one or more regulatory sequences, such as, for example, a strong constitutive pol III, e.g., human or mouse U6 snRNA promoter, the human and mouse H1 RNA promoter, or the human tRNA-val promoter, or a strong constitutive pol II promoter, as described elsewhere herein.

The polynucleotides contemplated in particular embodiments, regardless of the length of the coding sequence itself, may be combined with other DNA sequences, such as promoters and/or enhancers, untranslated regions (UTRs), Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, post-transcription response elements, and polynucleotides encoding self-cleaving polypeptides, epitope tags, as disclosed elsewhere herein or as known in the art, such that their overall length may vary considerably. It is therefore contemplated in particular embodiments that a polynucleotide fragment of almost any length may be employed, with the total length preferably being limited by the ease of preparation and use in the intended recombinant DNA protocol.

Polynucleotides can be prepared, manipulated, expressed and/or delivered using any of a variety of well-established techniques known and available in the art. In order to express a desired polypeptide, a nucleotide sequence encoding the polypeptide, can be inserted into appropriate vector. A desired polypeptide can also be expressed by delivering an mRNA encoding the polypeptide into the cell.

Illustrative examples of vectors include, but are not limited to plasmid, autonomously replicating sequences, and transposable elements, e.g., Sleeping Beauty, PiggyBac.

Additional illustrative examples of vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses.

Illustrative examples of viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40).

Illustrative examples of expression vectors include, but are not limited to pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. In particular embodiments, coding sequences of polypeptides disclosed herein can be ligated into such expression vectors for the expression of the polypeptides in mammalian cells.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally.

“Expression control sequences,” “control elements,” or “regulatory sequences” present in an expression vector are those non-translated regions of the vector-origin of replication, selection cassettes, promoters, enhancers, translation initiation signals (Shine Dalgamo sequence or Kozak sequence) introns, post-transcriptional regulatory elements, a polyadenylation sequence, 5′ and 3′ untranslated regions-which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In particular embodiments, a polynucleotide comprises a vector, including but not limited to expression vectors and viral vectors. A vector may comprise one or more exogenous, endogenous, or heterologous control sequences such as promoters and/or enhancers. An “endogenous control sequence” is one which is naturally linked with a given gene in the genome. An “exogenous control sequence” is one which is placed in juxtaposition to a gene by means of genetic manipulation (i.e., molecular biological techniques) such that transcription of that gene is directed by the linked enhancer/promoter. A “heterologous control sequence” is an exogenous sequence that is from a different species than the cell being genetically manipulated. A “synthetic” control sequence may comprise elements of one more endogenous and/or exogenous sequences, and/or sequences determined in vitro or in silico that provide optimal promoter and/or enhancer activity for the particular therapy.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, a short elongation factor 1-alpha (EF1a-short) promoter, a long elongation factor 1-alpha (EF1a-long) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), 0-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, d1587rev primer-binding site substituted (MND) promoter (Challita et al., J Virol. 69(2):748-55 (1995)).

In a particular embodiment, it may be desirable to use a cell, cell type, cell lineage or tissue specific expression control sequence to achieve cell type specific, lineage specific, or tissue specific expression of a desired polynucleotide sequence (e.g., to express a particular nucleic acid encoding a polypeptide in only a subset of cell types, cell lineages, or tissues or during specific stages of development).

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Conditional expression can also be achieved by using a site specific DNA recombinase. According to certain embodiments, polynucleotides comprise at least one (typically two) site(s) for recombination mediated by a site specific recombinase. As used herein, the terms “recombinase” or “site specific recombinase” include excisive or integrative proteins, enzymes, co-factors or associated proteins that are involved in recombination reactions involving one or more recombination sites (e.g., two, three, four, five, six, seven, eight, nine, ten or more.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases suitable for use in particular embodiments include, but are not limited to: Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

The polynucleotides may comprise one or more recombination sites for any of a wide variety of site specific recombinases. It is to be understood that the target site for a site specific recombinase is in addition to any site(s) required for integration of a vector, e.g., a retroviral vector or lentiviral vector. As used herein, the terms “recombination sequence,” “recombination site,” or “site specific recombination site” refer to a particular nucleic acid sequence to which a recombinase recognizes and binds.

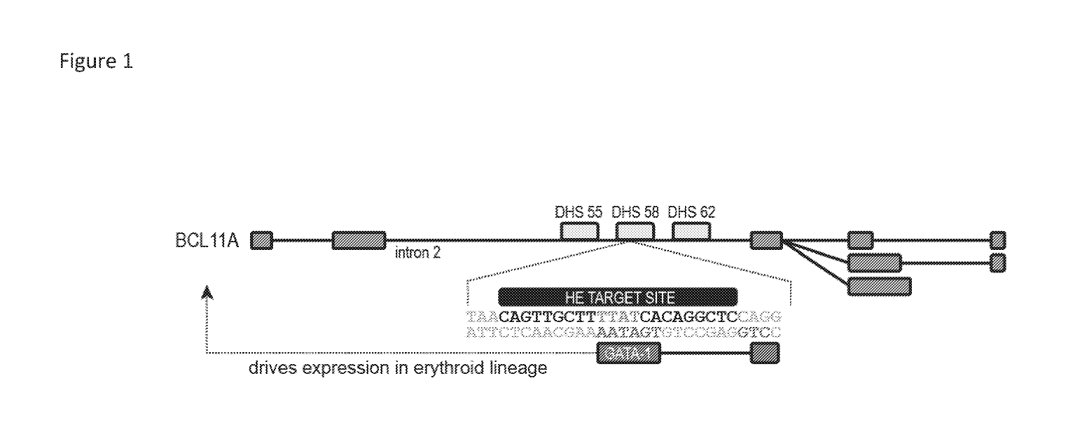

For example, one recombination site for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Other exemplary loxP sites include, but are not limited to: lox511 (Hoess et al., 1996; Bethke and Sauer, 1997), lox5171 (Lee and Saito, 1998), lox2272 (Lee and Saito, 1998), m2 (Langer et al., 2002), lox71 (Albert et al., 1995), and lox66 (Albert et al., 1995).

Suitable recognition sites for the FLP recombinase include, but are not limited to: FRT (McLeod, et al., 1996), F₁, F₂, F₃ (Schlake and Bode, 1994), F₄, F₅ (Schlake and Bode, 1994), FRT(LE) (Senecoff et al., 1988), FRT(RE) (Senecoff et al., 1988).

Other examples of recognition sequences are the attB, attP, attL, and attR sequences, which are recognized by the recombinase enzyme λ Integrase, e.g., phi-c31. The φC31 SSR mediates recombination only between the heterotypic sites attB (34 bp in length) and attP (39 bp in length) (Groth et al., 2000). attB and attP, named for the attachment sites for the phage integrase on the bacterial and phage genomes, respectively, both contain imperfect inverted repeats that are likely bound by φC31 homodimers (Groth et al., 2000). The product sites, attL and attR, are effectively inert to further φC31-mediated recombination (Belteki et al., 2003), making the reaction irreversible. For catalyzing insertions, it has been found that attB-bearing DNA inserts into a genomic attP site more readily than an attP site into a genomic attB site (Thyagarajan et al., 2001; Belteki et al., 2003). Thus, typical strategies position by homologous recombination an attP-bearing “docking site” into a defined locus, which is then partnered with an attB-bearing incoming sequence for insertion.

In one embodiment, a polynucleotide contemplated herein comprises a donor repair template polynucleotide flanked by a pair of recombinase recognition sites. In particular embodiments, the repair template polynucleotide is flanked by LoxP sites, FRT sites, or att sites.

In particular embodiments, polynucleotides contemplated herein, include one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. Examples of IRES generally employed by those of skill in the art include those described in U.S. Pat. No. 6,692,736. Further examples of “IRES” known in the art include, but are not limited to IRES obtainable from picomavirus (Jackson et al., 1990) and IRES obtainable from viral or cellular mRNA sources, such as for example, immunoglobulin heavy-chain binding protein (BiP), the vascular endothelial growth factor (VEGF) (Huez et al. 1998. Mol. Cell. Biol. 18(11):6178-6190), the fibroblast growth factor 2 (FGF-2), and insulin-like growth factor (IGFII), the translational initiation factor eIF4G and yeast transcription factors TFIID and HAP4, the encephelomycarditis virus (EMCV) which is commercially available from Novagen (Duke et al., 1992. J. Virol 66(3): 1602-9) and the VEGF IRES (Huez et al., 1998. Mol Cell Biol 18(11):6178-90). IRES have also been reported in viral genomes of Picomaviridae, Dicistroviridae and Flaviviridae species and in HCV, Friend murine leukemia virus (FrMLV) and Moloney murine leukemia virus (MoMLV).

In one embodiment, the IRES used in polynucleotides contemplated herein is an EMCV IRES.

In particular embodiments, the polynucleotides comprise polynucleotides that have a consensus Kozak sequence and that encode a desired polypeptide. As used herein, the term “Kozak sequence” refers to a short nucleotide sequence that greatly facilitates the initial binding of mRNA to the small subunit of the ribosome and increases translation. The consensus Kozak sequence is (GCC)RCCATGG (SEQ ID NO:76), where R is a purine (A or G) (Kozak, 1986. Cell. 44(2):283-92, and Kozak, 1987. Nucleic Acids Res. 15(20):8125-48).

Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed. The terms “polyA site,” “polyA sequence,” “poly(A) site” or “poly(A) sequence” as used herein denote a DNA sequence which directs both the termination and polyadenylation of the nascent RNA transcript by RNA polymerase II. Polyadenylation sequences can promote mRNA stability by addition of a poly(A) tail to the 3′ end of the coding sequence and thus, contribute to increased translational efficiency. Efficient polyadenylation of the recombinant transcript is desirable as transcripts lacking a poly(A) tail are unstable and are rapidly degraded. Illustrative examples of poly(A) signals that can be used in a vector, includes an ideal poly(A) sequence (e.g., AATAAA, ATTAAA, AGTAAA), a bovine growth hormone poly(A) sequence (BGHpA), a rabbit β-globin poly(A) sequence (rβgpA), or another suitable heterologous or endogenous poly(A) sequence known in the art.

In some embodiments, a polynucleotide or cell harboring the polynucleotide utilizes a suicide gene, including an inducible suicide gene to reduce the risk of direct toxicity and/or uncontrolled proliferation. In specific embodiments, the suicide gene is not immunogenic to the host harboring the polynucleotide or cell. A certain example of a suicide gene that may be used is caspase-9 or caspase-8 or cytosine deaminase. Caspase-9 can be activated using a specific chemical inducer of dimerization (CID).

In certain embodiments, polynucleotides comprise gene segments that cause the genetically modified cells contemplated herein to be susceptible to negative selection in vivo. “Negative selection” refers to an infused cell that can be eliminated as a result of a change in the in vivo condition of the individual. The negative selectable phenotype may result from the insertion of a gene that confers sensitivity to an administered agent, for example, a compound. Negative selection genes are known in the art, and include, but are not limited to: the Herpes simplex virus type I thymidine kinase (HSV-I TK) gene which confers ganciclovir sensitivity; the cellular hypoxanthine phosphribosyltransferase (HPRT) gene, the cellular adenine phosphoribosyltransferase (APRT) gene, and bacterial cytosine deaminase.

In some embodiments, genetically modified cells comprise a polynucleotide further comprising a positive marker that enables the selection of cells of the negative selectable phenotype in vitro. The positive selectable marker may be a gene, which upon being introduced into the host cell, expresses a dominant phenotype permitting positive selection of cells carrying the gene. Genes of this type are known in the art, and include, but are not limited to hygromycin-B phosphotransferase gene (hph) which confers resistance to hygromycin B, the amino glycoside phosphotransferase gene (neo or aph) from Tn5 which codes for resistance to the antibiotic G418, the dihydrofolate reductase (DHFR) gene, the adenosine deaminase gene (ADA), and the multi-drug resistance (MDR) gene.

In one embodiment, the positive selectable marker and the negative selectable element are linked such that loss of the negative selectable element necessarily also is accompanied by loss of the positive selectable marker. In a particular embodiment, the positive and negative selectable markers are fused so that loss of one obligatorily leads to loss of the other. An example of a fused polynucleotide that yields as an expression product a polypeptide that confers both the desired positive and negative selection features described above is a hygromycin phosphotransferase thymidine kinase fusion gene (HyTK). Expression of this gene yields a polypeptide that confers hygromycin B resistance for positive selection in vitro, and ganciclovir sensitivity for negative selection in vivo. See also the publications of PCT US91/08442 and PCT/US94/05601, by S. D. Lupton, describing the use of bifunctional selectable fusion genes derived from fusing a dominant positive selectable markers with negative selectable markers.

Preferred positive selectable markers are derived from genes selected from the group consisting of hph, nco, and gpt, and preferred negative selectable markers are derived from genes selected from the group consisting of cytosine deaminase, HSV-I TK, VZV TK, HPRT, APRT and gpt. Exemplary bifunctional selectable fusion genes contemplated in particular embodiments include, but are not limited to genes wherein the positive selectable marker is derived from hph or neo, and the negative selectable marker is derived from cytosine deaminase or a TK gene or selectable marker.

In particular embodiments, polynucleotides encoding one or more homing endonuclease variants, megaTALs, end-processing enzymes, or fusion polypeptides may be introduced into hematopoietic cells, e.g., CD34⁺ cells, by both non-viral and viral methods. In particular embodiments, delivery of one or more polynucleotides encoding nucleases and/or donor repair templates may be provided by the same method or by different methods, and/or by the same vector or by different vectors.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In particular embodiments, non-viral vectors are used to deliver one or more polynucleotides contemplated herein to a CD34⁺ cell.

Illustrative examples of non-viral vectors include, but are not limited to plasmids (e.g., DNA plasmids or RNA plasmids), transposons, cosmids, and bacterial artificial chromosomes.

Illustrative methods of non-viral delivery of polynucleotides contemplated in particular embodiments include, but are not limited to: electroporation, sonoporation, lipofection, microinjection, biolistics, virosomes, liposomes, immunoliposomes, nanoparticles, polycation or lipid:nucleic acid conjugates, naked DNA, artificial virions, DEAE-dextran-mediated transfer, gene gun, and heat-shock.

Illustrative examples of polynucleotide delivery systems suitable for use in particular embodiments contemplated in particular embodiments include, but are not limited to those provided by Amaxa Biosystems, Maxcyte, Inc., BTX Molecular Delivery Systems, and Copernicus Therapeutics Inc. Lipofection reagents are sold commercially (e.g., Transfectam™ and Lipofectin™). Cationic and neutral lipids that are suitable for efficient receptor-recognition lipofection of polynucleotides have been described in the literature. See e.g., Liu et al. (2003) Gene Therapy. 10:180-187; and Balazs et al. (2011) Journal of Drug Delivery. 2011:1-12. Antibody-targeted, bacterially derived, non-living nanocell-based delivery is also contemplated in particular embodiments.

Viral vectors comprising polynucleotides contemplated in particular embodiments can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., mobilized peripheral blood, lymphocytes, bone marrow aspirates, tissue biopsy, etc.) or universal donor hematopoietic stem cells, followed by reimplantation of the cells into a patient.

In one embodiment, viral vectors comprising nuclease variants and/or donor repair templates are administered directly to an organism for transduction of cells in vivo. Alternatively, naked DNA or mRNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Illustrative examples of viral vector systems suitable for use in particular embodiments contemplated herein include, but are not limited to adeno-associated virus (AAV), retrovirus, herpes simplex virus, adenovirus, and vaccinia virus vectors.

In various embodiments, one or more polynucleotides encoding a nuclease variant and/or donor repair template are introduced into a hematopoietic cell, e.g., a hematopoietic stem or progenitor cell, or CD34⁺ cell, by transducing the cell with a recombinant adeno-associated virus (rAAV), comprising the one or more polynucleotides.

AAV is a small (˜26 nm) replication-defective, primarily episomal, non-enveloped virus. AAV can infect both dividing and non-dividing cells and may incorporate its genome into that of the host cell. Recombinant AAV (rAAV) are typically composed of, at a minimum, a transgene and its regulatory sequences, and 5′ and 3′ AAV inverted terminal repeats (ITRs). The ITR sequences are about 145 bp in length. In particular embodiments, the rAAV comprises ITRs and capsid sequences isolated from AAV1, AAV2, AAV3, AAV4, AAV5, AAV6, AAV7, AAV8, AAV9, or AAV10.

In some embodiments, a chimeric rAAV is used the ITR sequences are isolated from one AAV serotype and the capsid sequences are isolated from a different AAV serotype. For example, a rAAV with ITR sequences derived from AAV2 and capsid sequences derived from AAV6 is referred to as AAV2/AAV6. In particular embodiments, the rAAV vector may comprise ITRs from AAV2, and capsid proteins from any one of AAV1, AAV2, AAV3, AAV4, AAV5, AAV6, AAV7, AAV8, AAV9, or AAV10. In a preferred embodiment, the rAAV comprises ITR sequences derived from AAV2 and capsid sequences derived from AAV6. In a preferred embodiment, the rAAV comprises ITR sequences derived from AAV2 and capsid sequences derived from AAV2.

In some embodiments, engineering and selection methods can be applied to AAV capsids to make them more likely to transduce cells of interest.

Construction of rAAV vectors, production, and purification thereof have been disclosed, e.g., in U.S. Pat. Nos. 9,169,494; 9,169,492; 9,012,224; 8,889,641; 8,809,058; and 8,784,799, each of which is incorporated by reference herein, in its entirety.

In various embodiments, one or more polynucleotides encoding a nuclease variant and/or donor repair template are introduced into a hematopoietic cell, e.g., a hematopoietic stem or progenitor cell, or CD34⁺ cell, by transducing the cell with a retrovirus, e.g., lentivirus, comprising the one or more polynucleotides. In one embodiment, a nuclease variant and/or donor repair template are introduced into a hematopoietic cell, e.g., a hematopoietic stem or progenitor cell, or CD34⁺ cell, by transducing the cell with an integrase deficient lentivirus.

As used herein, the term “retrovirus” refers to an RNA virus that reverse transcribes its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

In various embodiments, a lentiviral vector contemplated herein comprises one or more LTRs, and one or more, or all, of the following accessory elements: a cPPT/FLAP, a Psi (Ψ) packaging signal, an export element, poly (A) sequences, and may optionally comprise a WPRE or HPRE, an insulator element, a selectable marker, and a cell suicide gene, as discussed elsewhere herein.

In particular embodiments, lentiviral vectors contemplated herein may be integrative or non-integrating or integration defective lentivirus. As used herein, the term “integration defective lentivirus” or “IDLV” refers to a lentivirus having an integrase that lacks the capacity to integrate the viral genome into the genome of the host cells. Integration-incompetent viral vectors have been described in patent application WO 2006/010834, which is herein incorporated by reference in its entirety.

Illustrative mutations in the HIV-1 pol gene suitable to reduce integrase activity include, but are not limited to: H12N, H12C, H16C, H16V, S81R, D41A, K42A, H51A, Q53C, D55V, D64E, D64V, E69A, K71A, E85A, E87A, D116N, D1161, D116A, N120G, N1201, N120E, E152G, E152A, D35E, K156E, K156A, E157A, K159E, K159A, K160A, R166A, D167A, E170A, H171A, K173A, K186Q, K186T, K188T, E198A, R199c, R199T, R199A, D202A, K211A, Q214L, Q216L, Q221 L, W235F, W235E, K236S, K236A, K246A, G247W, D253A, R262A, R263A and K264H.

In one embodiment, the HIV-1 integrase deficient pol gene comprises a D64V, D1161, D116A, E152G, or E152A mutation; D64V, D1161, and E152G mutations; or D64V, D116A, and E152A mutations.

In one embodiment, the HIV-1 integrase deficient pol gene comprises a D64V mutation.

The term “long terminal repeat (LTR)” refers to domains of base pairs located at the ends of retroviral DNAs which, in their natural sequence context, are direct repeats and contain U3, R and U5 regions.

As used herein, the term “FLAP element” or “cPPT/FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173. In another embodiment, a lentiviral vector contains a FLAP element with one or more mutations in the cPPT and/or CTS elements. In yet another embodiment, a lentiviral vector comprises either a cPPT or CTS element. In yet another embodiment, a lentiviral vector does not comprise a cPPT or CTS element.

As used herein, the term “packaging signal” or “packaging sequence” refers to psi [F] sequences located within the retroviral genome which are required for insertion of the viral RNA into the viral capsid or particle, see e.g., Clever et al., 1995. J. of Virology, Vol. 69, No. 4; pp. 2101-2109.

The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J Virol. 65: 1053; and Cullen et al., 1991. Cell 58: 423), and the hepatitis B virus post-transcriptional regulatory element (HPRE).

In particular embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766).

Lentiviral vectors preferably contain several safety enhancements as a result of modifying the LTRs. “Self-inactivating” (SIN) vectors refers to replication-defective vectors, e.g., in which the right (3′) LTR enhancer-promoter region, known as the U3 region, has been modified (e.g., by deletion or substitution) to prevent viral transcription beyond the first round of viral replication. An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters.

The terms “pseudotype” or “pseudotyping” as used herein, refer to a virus whose viral envelope proteins have been substituted with those of another virus possessing preferable characteristics. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4⁺ presenting cells.

In certain embodiments, lentiviral vectors are produced according to known methods. See e.g., Kutner et al., BMC Biotechnol. 2009; 9:10. doi: 10.1186/1472-6750-9-10; Kutner et al. Nat. Protoc. 2009; 4(4):495-505. doi: 10.1038/nprot.2009.22.

According to certain specific embodiments contemplated herein, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid contemplated herein.

In various embodiments, one or more polynucleotides encoding a nuclease variant and/or donor repair template are introduced into a hematopoietic cell, e.g., a hematopoietic stem or progenitor cell, or CD34⁺ cell, by transducing the cell with an adenovirus comprising the one or more polynucleotides.

Adenoviral based vectors are capable of very high transduction efficiency in many cell types and do not require cell division. With such vectors, high titer and high levels of expression have been obtained. This vector can be produced in large quantities in a relatively simple system. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity.

Generation and propagation of the current adenovirus vectors, which are replication deficient, may utilize a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones & Shenk, 1978), the current adenovirus vectors, with the help of 293 cells, carry foreign DNA in either the E1, the D3 or both regions (Graham & Prevec, 1991). Adenovirus vectors have been used in eukaryotic gene expression (Levrero et al., 1991; Gomez-Foix et al., 1992) and vaccine development (Grunhaus & Horwitz, 1992; Graham & Prevec, 1992). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993). An example of the use of an Ad vector in a clinical trial involved polynucleotide therapy for antitumor immunization with intramuscular injection (Sterman et al., Hum. Gene Ther. 7:1083-9 (1998)).

In various embodiments, one or more polynucleotides encoding a nuclease variant and/or donor repair template are introduced into a hematopoietic cell, e.g., a hematopoietic stem or progenitor cell, or CD34⁺ cell, by transducing the cell with a herpes simplex virus, e.g., HSV-1, HSV-2, comprising the one or more polynucleotides.

The mature HSV virion consists of an enveloped icosahedral capsid with a viral genome consisting of a linear double-stranded DNA molecule that is 152 kb. In one embodiment, the HSV based viral vector is deficient in one or more essential or non-essential HSV genes. In one embodiment, the HSV based viral vector is replication deficient. Most replication deficient HSV vectors contain a deletion to remove one or more intermediate-early, early, or late HSV genes to prevent replication. For example, the HSV vector may be deficient in an immediate early gene selected from the group consisting of: ICP4, ICP22, ICP27, ICP47, and a combination thereof. Advantages of the HSV vector are its ability to enter a latent stage that can result in long-term DNA expression and its large viral DNA genome that can accommodate exogenous DNA inserts of up to 25 kb. HSV-based vectors are described in, for example, U.S. Pat. Nos. 5,837,532, 5,846,782, and 5,804,413, and International Patent Applications WO 91/02788, WO 96/04394, WO 98/15637, and WO 99/06583, each of which are incorporated by reference herein in its entirety.

### H. Genome Edited Cells

The genome edited cells manufactured by the methods contemplated in particular embodiments provide improved cell-based therapeutics for the treatment of hemoglobinopathies. Without wishing to be bound to any particular theory, it is believed that the compositions and methods contemplated herein co-opt fetal globin switching mechanisms to provide a more robust genome edited cell composition that may be used to treat, and in some embodiments potentially cure, hemoglobinopathies.

Genome edited cells contemplated in particular embodiments may be autologous/autogeneic (“self”) or non-autologous (“non-self,” e.g., allogeneic, syngeneic or xenogeneic). “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells are obtained from a mammalian subject. In a more preferred embodiment, the cells are obtained from a primate subject, optionally a non-human primate. In the most preferred embodiment, the cells are obtained from a human subject.

An “isolated cell” refers to a non-naturally occurring cell, e.g., a cell that does not exist in nature, a modified cell, an engineered cell, etc., that has been obtained from an in vivo tissue or organ and is substantially free of extracellular matrix.

Illustrative examples of cell types whose genome can be edited using the compositions and methods contemplated herein include, but are not limited to, cell lines, primary cells, stem cells, progenitor cells, and differentiated cells.

The term “stem cell” refers to a cell which is an undifferentiated cell capable of (1) long term self-renewal, or the ability to generate at least one identical copy of the original cell, (2) differentiation at the single cell level into multiple, and in some instance only one, specialized cell type and (3) of in vivo functional regeneration of tissues. Stem cells are subclassified according to their developmental potential as totipotent, pluripotent, multipotent and oligo/unipotent. “Self-renewal” refers a cell with a unique capacity to produce unaltered daughter cells and to generate specialized cell types (potency). Self-renewal can be achieved in two ways. Asymmetric cell division produces one daughter cell that is identical to the parental cell and one daughter cell that is different from the parental cell and is a progenitor or differentiated cell. Symmetric cell division produces two identical daughter cells. “Proliferation” or “expansion” of cells refers to symmetrically dividing cells.

As used herein, the term “progenitor” or “progenitor cells” refers to cells have the capacity to self-renew and to differentiate into more mature cells. Many progenitor cells differentiate along a single lineage, but may have quite extensive proliferative capacity.

In particular embodiments, the cell is a primary cell. The term “primary cell” as used herein is known in the art to refer to a cell that has been isolated from a tissue and has been established for growth in vitro or ex vivo. Corresponding cells have undergone very few, if any, population doublings and are therefore more representative of the main functional component of the tissue from which they are derived in comparison to continuous cell lines, thus representing a more representative model to the in vivo state. Methods to obtain samples from various tissues and methods to establish primary cell lines are well-known in the art (see, e.g., Jones and Wise, Methods Mol Biol. 1997). Primary cells for use in the methods contemplated herein are derived from umbilical cord blood, placental blood, mobilized peripheral blood and bone marrow. In one embodiment, the primary cell is a hematopoietic stem or progenitor cell.

In one embodiment, the genome edited cell is an embryonic stem cell.

In one embodiment, the genome edited cell is an adult stem or progenitor cell.

In one embodiment, the genome edited cell is primary cell.

In a preferred embodiment, the genome edited cell is a hematopoietic cell, e.g, hematopoietic stem cell, hematopoietic progenitor cell, an erythroid cell, or cell population comprising hematopoietic cells.

As used herein, the term “population of cells” refers to a plurality of cells that may be made up of any number and/or combination of homogenous or heterogeneous cell types, as described elsewhere herein. For example, for transduction of hematopoietic stem or progenitor cells, a population of cells may be isolated or obtained from umbilical cord blood, placental blood, bone marrow, or mobilized peripheral blood. A population of cells may comprise about 10%, about 20%, about 30%, about 40%, about 50%, about 60%, about 70%, about 80%, about 90%, or about 100% of the target cell type to be edited. In certain embodiments, hematopoietic stem or progenitor cells may be isolated or purified from a population of heterogeneous cells using methods known in the art.

Illustrative sources to obtain hematopoietic cells include, but are not limited to: cord blood, bone marrow or mobilized peripheral blood.

Hematopoietic stem cells (HSCs) give rise to committed hematopoietic progenitor cells (HPCs) that are capable of generating the entire repertoire of mature blood cells over the lifetime of an organism. The term “hematopoietic stem cell” or “HSC” refers to multipotent stem cells that give rise to the all the blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells), and others known in the art (See Fei, R., et al., U.S. Pat. No. 5,635,387; McGlave, et al., U.S. Pat. No. 5,460,964; Simmons, P., et al., U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al., U.S. Pat. No. 5,716,827). When transplanted into lethally irradiated animals or humans, hematopoietic stem and progenitor cells can repopulate the erythroid, neutrophil-macrophage, megakaryocyte and lymphoid hematopoietic cell pool.

Additional illustrative examples of hematopoietic stem or progenitor cells suitable for use with the methods and compositions contemplated herein include hematopoietic cells that are CD34⁺CD38^{Lo}CD90⁺CD45^{RA-}, hematopoietic cells that are CD34⁺, CD59⁺, Thy1/CD90⁺, CD38^{Lo/-}, C-kit/CD117⁺, and Lin⁽⁻⁾, and hematopoietic cells that are CD133⁺.

In a preferred embodiment, the hematopoietic cells that are CD133⁺CD90⁺.

In a preferred embodiment, the hematopoietic cells that are CD133⁺CD34⁺.

In a preferred embodiment, the hematopoietic cells that are CD133⁺CD90+CD34⁺.

Various methods exist to characterize hematopoietic hierarchy. One method of characterization is the SLAM code. The SLAM (Signaling lymphocyte activation molecule) family is a group of >10 molecules whose genes are located mostly tandemly in a single locus on chromosome 1 (mouse), all belonging to a subset of immunoglobulin gene superfamily, and originally thought to be involved in T-cell stimulation. This family includes CD48, CD150, CD244, etc., CD150 being the founding member, and, thus, also called slamF1, i.e., SLAM family member 1. The signature SLAM code for the hematopoietic hierarchy is hematopoietic stem cells (HSC)—CD150⁺CD48⁻CD244⁻; multipotent progenitor cells (MPPs)—CD150⁻CD48⁻CD244⁺; lineage-restricted progenitor cells (LRPs)—CD150-CD48+CD244+; common myeloid progenitor (CMP)—lin⁻SCA-1-c-kit⁺CD34⁺CD16/32^{mid}; granulocyte-macrophage progenitor (GMP)—lin⁻SCA-1-c-kit⁺CD34⁺CD16/32^{hi}; and megakaryocyte-erythroid progenitor (MEP)—lin⁻SCA-1-c-kit⁺CD34⁻CD16/32^{low}.

Preferred target cell types edited with the compositions and methods contemplated herein include, hematopoietic cells, preferably human hematopoietic cells, more preferably human hematopoietic stem and progenitor cells, and even more preferably CD34⁺ human hematopoietic stem cells. The term “CD34+ cell,” as used herein refers to a cell expressing the CD34 protein on its cell surface. “CD34,” as used herein refers to a cell surface glycoprotein (e.g., sialomucin protein) that often acts as a cell-cell adhesion factor. CD34+ is a cell surface marker of both hematopoietic stem and progenitor cells.

In one embodiment, the genome edited hematopoietic cells are CD150⁺CD48⁻CD244⁻ cells.

In one embodiment, the genome edited hematopoietic cells are CD34⁺CD133⁺ cells.

In one embodiment, the genome edited hematopoietic cells are CD133⁺ cells.

In one embodiment, the genome edited hematopoietic cells are CD34⁺ cells.

In particular embodiments, a population of hematopoietic cells comprising hematopoietic stem and progenitor cells (HSPCs) comprises an edited BCL11A gene, wherein the edit is a DSB repaired by NHEJ. The edit may be in an erythroid specific enhancer in the BCL11A gene, preferably in a GATA-1 binding site in the BCL11A gene, and more preferably in a consensus GATA-1 binding site in the second intron of the BCL11A gene.

In particular embodiments, a population of hematopoietic cells comprising hematopoietic stem and progenitor cells (HSPCs) comprises an edited BCL11A gene comprising an insertion or deletion (INDEL) of about 1, 2, 3, 4, 5, 6, 7, 8, 9, 10, 11, 12, 13, 14 15, 16, 17, 18, 19, 20, 21, 22, 23, 24, 25 or more nucleotides in an erythroid specific enhancer in the BCL11A gene, preferably in a GATA-1 binding site in the BCL11A gene, more preferably in a consensus GATA-1 binding site in the second intron of the BCL11A gene, and even more preferably in a target site set forth in SEQ ID NO: 25 (the complement of which includes the Consensus GATA-1 motif WGATAR); thereby decreasing, reducing, or ablating BCL11A expression.

In one embodiment, the edit is an insertion of 1 nucleotide or a deletion of about 1, 2, 3, or 4 nucleotides in an erythroid specific enhancer in the BCL11A gene, preferably in a GATA-1 binding site in the BCL11A gene, more preferably in a consensus GATA-1 binding site in the second intron of the BCL11A gene, and even more preferably in a target site set forth in SEQ ID NO: 25 (the complement of which includes the Consensus GATA-1 motif WGATAR); thereby decreasing, reducing, or ablating BCL11A expression.

In particular embodiments, the genome edited cells comprise erythroid cells.

In particular embodiments, the genome edited cells comprise one or more mutations in a β-globin gene. In one embodiment, the β-globin alleles of the subject are selected from the group consisting of: β^{E}/β⁰, β^{C}/β⁰, β⁰/β⁰, β^{E}/β^{E}, β^{C}/β⁺, β^{E}/β⁺, β⁰/β⁺, β⁺/β⁺, β^{C}/β^{C}, β^{E}/β^{S}, β⁰/β^{S}, β^{C}/β^{S}, β⁺/β^{S} or β^{S}/β^{S}.

In particular embodiments, the genome edited cells comprise one or more one or more mutations in a β-globin gene that result in a thalassemia. In one embodiment, the thalassemia is an α-thalassemia. In one embodiment, the thalassemia is a β-thalassemia. In one embodiment, the β-globin alleles of the subject are selected from the group consisting of: β^{E}/β⁰, β^{C}/β⁰, β⁰/β⁰, β^{C}/β^{C}, β^{E}/β^{E}, β^{E}/β⁺, β^{C}/β^{E}, β^{C}/β⁺, β⁰/β⁺, or β⁺/β⁺.

In particular embodiments, the genome edited cells comprise one or more one or more mutations in a β-globin gene that result in sickle cell disease. In one embodiment, the β-globin alleles of the subject are selected from the group consisting of: β^{E}/β^{S}, β⁰/β^{S}, β^{C}/β^{S}, β⁺/β^{S} or β^{S}/β^{S}.

### I. Compositions and Formulations

The compositions contemplated in particular embodiments may comprise one or more polypeptides, polynucleotides, vectors comprising same, and genome editing compositions and genome edited cell compositions, as contemplated herein. The genome editing compositions and methods contemplated in particular embodiments are useful for editing a target site in the human BCL11A gene in a cell or a population of cells. In preferred embodiments, a genome editing composition is used to edit a BCL11A gene in a hematopoietic cell, e.g., a hematopoietic stem or progenitor cell, or a CD34⁺ cell.

In various embodiments, the compositions contemplated herein comprise a nuclease variant, and optionally an end-processing enzyme, e.g., a 3′-5′ exonuclease (Trex2). The nuclease variant may be in the form of an mRNA that is introduced into a cell via polynucleotide delivery methods disclosed supra, e.g., electroporation, lipid nanoparticles, etc. In one embodiment, a composition comprising an mRNA encoding a homing endonuclease variant or megaTAL, and optionally a 3′-5′ exonuclease, is introduced in a cell via polynucleotide delivery methods disclosed supra. The composition may be used to generate a genome edited cell or population of genome edited cells by error prone NHEJ.

In particular embodiments, the compositions contemplated herein comprise a population of cells, a nuclease variant, and optionally, a donor repair template. In particular embodiments, the compositions contemplated herein comprise a population of cells, a nuclease variant, an end-processing enzyme, and optionally, a donor repair template. The nuclease variant and/or end-processing enzyme may be in the form of an mRNA that is introduced into the cell via polynucleotide delivery methods disclosed supra.

In particular embodiments, the compositions contemplated herein comprise a population of cells, a homing endonuclease variant or megaTAL, and optionally, a donor repair template. In particular embodiments, the compositions contemplated herein comprise a population of cells, a homing endonuclease variant or megaTAL, a 3′-5′ exonuclease, and optionally, a donor repair template. The homing endonuclease variant, megaTAL, and/or 3′-5′ exonuclease may be in the form of an mRNA that is introduced into the cell via polynucleotide delivery methods disclosed supra.

In particular embodiments, the population of cells comprise genetically modified hematopoietic cells including, but not limited to, hematopoietic stem cells, hematopoietic progenitor cells, CD133⁺ cells, and CD34⁺ cells.

Compositions include, but are not limited to pharmaceutical compositions. A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the composition.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” refers to a diluent, adjuvant, excipient, or vehicle with which the therapeutic cells are administered. Illustrative examples of pharmaceutical carriers can be sterile liquids, such as cell culture media, water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. Suitable pharmaceutical excipients in particular embodiments, include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Except insofar as any conventional media or agent is incompatible with the active ingredient, its use in the therapeutic compositions is contemplated. Supplementary active ingredients can also be incorporated into the compositions.

In one embodiment, a composition comprising a pharmaceutically acceptable carrier is suitable for administration to a subject. In particular embodiments, a composition comprising a carrier is suitable for parenteral administration, e.g., intravascular (intravenous or intraarterial), intraperitoneal or intramuscular administration. In particular embodiments, a composition comprising a pharmaceutically acceptable carrier is suitable for intraventricular, intraspinal, or intrathecal administration. Pharmaceutically acceptable carriers include sterile aqueous solutions, cell culture media, or dispersions. The use of such media and agents for pharmaceutically active substances is well known in the art. Except insofar as any conventional media or agent is incompatible with the transduced cells, use thereof in the pharmaceutical compositions is contemplated.

In particular embodiments, compositions contemplated herein comprise genetically modified hematopoietic stem and/or progenitor cells and a pharmaceutically acceptable carrier. A composition comprising a cell-based composition contemplated herein can be administered separately by enteral or parenteral administration methods or in combination with other suitable compounds to effect the desired treatment goals.

The pharmaceutically acceptable carrier must be of sufficiently high purity and of sufficiently low toxicity to render it suitable for administration to the human subject being treated. It further should maintain or increase the stability of the composition. The pharmaceutically acceptable carrier can be liquid or solid and is selected, with the planned manner of administration in mind, to provide for the desired bulk, consistency, etc., when combined with other components of the composition. For example, the pharmaceutically acceptable carrier can be, without limitation, a binding agent (e.g., pregelatinized maize starch, polyvinylpyrrolidone or hydroxypropyl methylcellulose, etc.), a filler (e.g., lactose and other sugars, microcrystalline cellulose, pectin, gelatin, calcium sulfate, ethyl cellulose, polyacrylates, calcium hydrogen phosphate, etc.), a lubricant (e.g., magnesium stearate, talc, silica, colloidal silicon dioxide, stearic acid, metallic stearates, hydrogenated vegetable oils, corn starch, polyethylene glycols, sodium benzoate, sodium acetate, etc.), a disintegrant (e.g., starch, sodium starch glycolate, etc.), or a wetting agent (e.g., sodium lauryl sulfate, etc.). Other suitable pharmaceutically acceptable carriers for the compositions contemplated herein include, but are not limited to, water, salt solutions, alcohols, polyethylene glycols, gelatins, amyloses, magnesium stearates, talcs, silicic acids, viscous paraffins, hydroxymethylcelluloses, polyvinylpyrrolidones and the like.

Such carrier solutions also can contain buffers, diluents and other suitable additives. The term “buffer” as used herein refers to a solution or liquid whose chemical makeup neutralizes acids or bases without a significant change in pH. Examples of buffers contemplated herein include, but are not limited to, Dulbecco's phosphate buffered saline (PBS), Ringer's solution, 5% dextrose in water (D5W), normal/physiologic saline (0.9% NaCl).

The pharmaceutically acceptable carriers may be present in amounts sufficient to maintain a pH of the composition of about 7. Alternatively, the composition has a pH in a range from about 6.8 to about 7.4, e.g., 6.8, 6.9, 7.0, 7.1, 7.2, 7.3, and 7.4. In still another embodiment, the composition has a pH of about 7.4.

Compositions contemplated herein may comprise a nontoxic pharmaceutically acceptable medium. The compositions may be a suspension. The term “suspension” as used herein refers to non-adherent conditions in which cells are not attached to a solid support. For example, cells maintained as a suspension may be stirred or agitated and are not adhered to a support, such as a culture dish.

In particular embodiments, compositions contemplated herein are formulated in a suspension, where the genome edited hematopoietic stem and/or progenitor cells are dispersed within an acceptable liquid medium or solution, e.g., saline or serum-free medium, in an intravenous (IV) bag or the like. Acceptable diluents include, but are not limited to water, PlasmaLyte, Ringer's solution, isotonic sodium chloride (saline) solution, serum-free cell culture medium, and medium suitable for cryogenic storage, e.g., Cryostor® medium.

In certain embodiments, a pharmaceutically acceptable carrier is substantially free of natural proteins of human or animal origin, and suitable for storing a composition comprising a population of genome edited cells, e.g., hematopoietic stem and progenitor cells. The therapeutic composition is intended to be administered into a human patient, and thus is substantially free of cell culture components such as bovine serum albumin, horse serum, and fetal bovine serum.

In some embodiments, compositions are formulated in a pharmaceutically acceptable cell culture medium. Such compositions are suitable for administration to human subjects. In particular embodiments, the pharmaceutically acceptable cell culture medium is a serum free medium.

Serum-free medium has several advantages over serum containing medium, including a simplified and better defined composition, a reduced degree of contaminants, elimination of a potential source of infectious agents, and lower cost. In various embodiments, the serum-free medium is animal-free, and may optionally be protein-free. Optionally, the medium may contain biopharmaceutically acceptable recombinant proteins. “Animal-free” medium refers to medium wherein the components are derived from non-animal sources. Recombinant proteins replace native animal proteins in animal-free medium and the nutrients are obtained from synthetic, plant or microbial sources. “Protein-free” medium, in contrast, is defined as substantially free of protein.

Illustrative examples of serum-free media used in particular compositions include, but are not limited to QBSF-60 (Quality Biological, Inc.), StemPro-34 (Life Technologies), and X-VIVO 10.

In a preferred embodiment, the compositions comprising genome edited hematopoietic stem and/or progenitor cells are formulated in PlasmaLyte.

In various embodiments, compositions comprising hematopoietic stem and/or progenitor cells are formulated in a cryopreservation medium. For example, cryopreservation media with cryopreservation agents may be used to maintain a high cell viability outcome post-thaw. Illustrative examples of cryopreservation media used in particular compositions include, but are not limited to, CryoStor CS10, CryoStor CS5, and CryoStor CS2.

In one embodiment, the compositions are formulated in a solution comprising 50:50 PlasmaLyte A to CryoStor CS10.

In particular embodiments, the composition is substantially free of mycoplasma, endotoxin, and microbial contamination. By “substantially free” with respect to endotoxin is meant that there is less endotoxin per dose of cells than is allowed by the FDA for a biologic, which is a total endotoxin of 5 EU/kg body weight per day, which for an average 70 kg person is 350 EU per total dose of cells. In particular embodiments, compositions comprising hematopoietic stem or progenitor cells transduced with a retroviral vector contemplated herein contains about 0.5 EU/mL to about 5.0 EU/mL, or about 0.5 EU/mL, 1.0 EU/mL, 1.5 EU/mL, 2.0 EU/mL, 2.5 EU/mL, 3.0 EU/mL, 3.5 EU/mL, 4.0 EU/mL, 4.5 EU/mL, or 5.0 EU/mL.

In certain embodiments, compositions and formulations suitable for the delivery of polynucleotides are contemplated including, but not limited to, one or more mRNAs encoding one or more reprogrammed nucleases, and optionally end-processing enzymes.

Exemplary formulations for ex vivo delivery may also include the use of various transfection agents known in the art, such as calcium phosphate, electroporation, heat shock and various liposome formulations (i.e., lipid-mediated transfection). Liposomes, as described in greater detail below, are lipid bilayers entrapping a fraction of aqueous fluid. DNA spontaneously associates to the external surface of cationic liposomes (by virtue of its charge) and these liposomes will interact with the cell membrane.

In particular embodiments, formulation of pharmaceutically-acceptable carrier solutions is well-known to those of skill in the art, as is the development of suitable dosing and treatment regimens for using the particular compositions described herein in a variety of treatment regimens, including e.g., enteral and parenteral, e.g., intravascular, intravenous, intraarterial, intraosseously, intraventricular, intracerebral, intracranial, intraspinal, intrathecal, and intramedullary administration and formulation. It would be understood by the skilled artisan that particular embodiments contemplated herein may comprise other formulations, such as those that are well known in the pharmaceutical art, and are described, for example, in Remington: The Science and Practice of Pharmacy, volume I and volume II. 22^{nd} Edition. Edited by Loyd V. Allen Jr. Philadelphia, Pa.: Pharmaceutical Press; 2012, which is incorporated by reference herein, in its entirety.

### J. Genome Edited Cell Therapies

The genome edited cells manufactured by the methods contemplated in particular embodiments provide improved drug products for use in the prevention, treatment, and amelioration of a hemoglobinopathy or for preventing, treating, or ameliorating at least one symptom associated with a hemoglobinopathy or a subject having a hemoglobinopathic mutation in a β-globin gene. As used herein, the term “drug product” refers to genetically modified cells produced using the compositions and methods contemplated herein. In particular embodiments, the drug product comprises genetically modified hematopoietic stem or progenitor cells, e.g., CD34⁺ cells. The genetically modified hematopoietic stem or progenitor cells give rise to adult erythroid cells with increased γ-globin gene expression and allow treatment of subjects having no or minimal expression of the γ-globin gene in vivo, thereby significantly expanding the opportunity to bring genome edited cell therapies to subjects for which this type of treatment was not previously a viable treatment option.

In particular embodiments, genome edited hematopoietic stem or progenitor cells comprise a non-functional or disrupted, ablated, or deleted erythroid specific enhancer in the BCL11A gene, thereby reducing or eliminating functional BCL11A expression in erythroid cells, e.g., insufficient BCL11A expression to repress or suppress γ-globin gene transcription and to transactivate β-globin gene transcription, and thereby increasing γ-globin gene expression in the erythroid cells.

In particular embodiments, genome edited hematopoietic stem or progenitor cells comprise a non-functional or disrupted, ablated, or deleted GATA-1 binding site in the BCL11A gene, preferably in a GATA-1 binding site in the BCL11A gene, more preferably in a consensus GATA-1 binding site in the second intron of the BCL11A gene, and even more preferably in a target site set forth in SEQ ID NO: 25 (the complement of which includes the Consensus GATA-1 motif WGATAR), thereby reducing or eliminating functional BCL11A expression in erythroid cells resulting in an increase in γ-globin gene expression in the erythroid cells.

In particular embodiments, genome edited hematopoietic stem or progenitor cells provide a curative, preventative, or ameliorative therapy to a subject diagnosed with or that is suspected of having monogenic disease, disorder, or condition or a disease, disorder, or condition of the hematopoietic system, e.g., a hemoglobinopathy.

As used herein, “hematopoiesis,” refers to the formation and development of blood cells from progenitor cells as well as formation of progenitor cells from stem cells. Blood cells include but are not limited to erythrocytes or red blood cells (RBCs), reticulocytes, monocytes, neutrophils, megakaryocytes, eosinophils, basophils, B-cells, macrophages, granulocytes, mast cells, thrombocytes, and leukocytes.

As used herein, the term “hemoglobinopathy” or “hemoglobinopathic condition” refers to a diverse group of inherited blood disorders that involve the presence of abnormal hemoglobin molecules resulting from alterations in the structure and/or synthesis of hemoglobin. Normally, hemoglobin consists of four protein subunits: two subunits of β-globin and two subunits of α-globin. Each of these protein subunits is attached (bound) to an iron-containing molecule called heme; each heme contains an iron molecule in its center that can bind to one oxygen molecule. Hemoglobin within red blood cells binds to oxygen molecules in the lungs. These cells then travel through the bloodstream and deliver oxygen to tissues throughout the body.

Hemoglobin A (HbA) is the designation for the normal hemoglobin that exists after birth. Hemoglobin A is a tetramer with two alpha chains and two beta chains (α₂β₂). Hemoglobin A2 is a minor component of the hemoglobin found in red cells after birth and consists of two alpha chains and two delta chains (α₂δ₂). Hemoglobin A2 generally comprises less than 3% of the total red cell hemoglobin. Hemoglobin F (HbF) is the predominant hemoglobin during fetal development. The molecule is a tetramer of two alpha chains and two gamma chains (α₂γ₂). In preferred embodiments, subjects are administered genome edited hematopoietic stem or progenitor cells that give rise to erythroid cells that have increased γ-globin gene expression and/or decreased hemoglobinopathic β-globin gene expression, thereby increasing the amount of HbF in the subject.

The most common hemoglobinopathies include sickle cell disease, (β-thalassemia, and α-thalassemia.

In particular embodiments, the compositions and methods contemplated herein provide genome edited cell therapies for subjects having a sickle cell disease. The term “sickle cell anemia” or “sickle cell disease” is defined herein to include any symptomatic anemic condition which results from sickling of red blood cells. Sickle cell anemia β^{S}/β^{S}, a common form of sickle cell disease (SCD), is caused by Hemoglobin S (HbS). HbS is generated by replacement of glutamic acid (E) with valine (V) at position 6 in β-globin, noted as Glu6Val or E6V. Replacing glutamic acid with valine causes the abnormal HbS subunits to stick together and form long, rigid molecules that bend red blood cells into a sickle (crescent) shape. The sickle-shaped cells die prematurely, which can lead to a shortage of red blood cells (anemia). In addition, the sickle-shaped cells are rigid and can block small blood vessels, causing severe pain and organ damage.

Additional mutations in the fl-globin gene can also cause other abnormalities in β-globin, leading to other types of sickle cell disease. These abnormal forms of β-globin are often designated by letters of the alphabet or sometimes by a name. In these other types of sickle cell disease, one β-globin subunit is replaced with HbS and the other β-globin subunit is replaced with a different abnormal variant, such as hemoglobin C (HbC; β-globin allele noted as β^{C}) or hemoglobin E (HbE; β-globin allele noted as β^{E}).

In hemoglobin SC (HbSC) disease, the β-globin subunits are replaced by HbS and HbC. HbC results from a mutation in the β-globin gene and is the predominant hemoglobin found in people with HbC disease (α₂β^{C}₂). HbC results when the amino acid lysine replaces the amino acid glutamic acid at position 6 in β-globin, noted as Glu6Lys or E6K. HbC disease is relatively benign, producing a mild hemolytic anemia and splenomegaly. The severity of HbSC disease is variable, but it can be as severe as sickle cell anemia.

HbE is caused when the amino acid glutamic acid is replaced with the amino acid lysine at position 26 in β-globin, noted as Glu26Lys or E26K. People with HbE disease have a mild hemolytic anemia and mild splenomegaly. HbE is extremely common in Southeast Asia and in some areas equals hemoglobin A in frequency. In some cases, the HbE mutation is present with HbS. In these cases, a person may have more severe signs and symptoms associated with sickle cell anemia, such as episodes of pain, anemia, and abnormal spleen function.

Other conditions, known as hemoglobin sickle-β-thalassemias (HbSBetaThal), are caused when mutations that produce hemoglobin S and β-thalassemia occur together. Mutations that combine sickle cell disease with beta-zero (β⁰; gene mutations that prevent β-globin production) thalassemia lead to severe disease, while sickle cell disease combined with beta-plus (β⁺; gene mutations that decrease β-globin production) thalassemia is milder.

As used herein, “thalassemia” refers to a hereditary disorder characterized by defective production of hemoglobin. Examples of thalassemias include α- and β-thalassemia.

In particular embodiments, the compositions and methods contemplated herein provide genome edited cell therapies for subjects having a β-thalassemia. β-thalassemias are caused by a mutation in the β-globin chain, and can occur in a major or minor form. Nearly 400 mutations in the β-globin gene have been found to cause β-thalassemia. Most of the mutations involve a change in a single DNA building block (nucleotide) within or near the β-globin gene. Other mutations insert or delete a small number of nucleotides in the β-globin gene. As noted above, β-globin gene mutations that decrease β-globin production result in a type of the condition called beta-plus (β⁺) thalassemia. Mutations that prevent cells from producing any beta-globin result in beta-zero (β⁰) thalassemia. In the major form of β-thalassemia, children are normal at birth, but develop anemia during the first year of life. The minor form of β-thalassemia produces small red blood cells. Thalassemia minor occurs if you receive the defective gene from only one parent. Persons with this form of the disorder are carriers of the disease and usually do not have symptoms.

HbE/β-thalassemia results from combination of HbE and β-thalassemia (β^{E}/β⁰, β^{E}/β⁺) and produces a condition more severe than is seen with either HbE trait or β-thalassemia trait. The disorder manifests as a moderately severe thalassemia that falls into the category of thalassemia intermedia. HbE/β-thalassemia is most common in people of Southeast Asian background.

In particular embodiments, the compositions and methods contemplated herein provide genome edited cell therapies for subjects having an α-thalassemia. α-thalassemia is a fairly common blood disorder worldwide. Thousands of infants with Hb Bart syndrome and HbH disease are born each year, particularly in Southeast Asia. α-thalassemia also occurs frequently in people from Mediterranean countries, North Africa, the Middle East, India, and Central Asia. α-thalassemia typically results from deletions involving the HBA1 and HBA2 genes. Both of these genes provide instructions for making a protein called α-globin, which is a component (subunit) of hemoglobin. People have two copies of the HBA1 gene and two copies of the HBA2 gene in each cell. The different types of α-thalassemia result from the loss of some or all of the HBA1 and HBA2 alleles.

Hb Bart syndrome, the most severe form of α-thalassemia, results from the loss of all four alpha-globin alleles. HbH disease is caused by a loss of three of the four α-globin alleles. In these two conditions, a shortage of α-globin prevents cells from making normal hemoglobin. Instead, cells produce abnormal forms of hemoglobin called hemoglobin Bart (Hb Bart) or hemoglobin H (HbH). These abnormal hemoglobin molecules cannot effectively carry oxygen to the body's tissues. The substitution of Hb Bart or HbH for normal hemoglobin causes anemia and the other serious health problems associated with α-thalassemia.

Two additional variants of α-thalassemia are related to a reduced amount of α-globin. Because cells still produce some normal hemoglobin, these variants tend to cause few or no health problems. A loss of two of the four α-globin alleles results in α-thalassemia trait. People with α-thalassemia trait may have unusually small, pale red blood cells and mild anemia. A loss of one α-globin allele is found in α-thalassemia silent carriers. These individuals typically have no thalassemia-related signs or symptoms.

In a preferred embodiment, genome edited cell therapies contemplated herein are used to treat, prevent, or ameliorate a hemoglobinopathy is selected from the group consisting of: hemoglobin C disease, hemoglobin E disease, sickle cell anemia, sickle cell disease (SCD), thalassemia, β-thalassemia, thalassemia major, thalassemia intermedia, α-thalassemia, hemoglobin Bart syndrome and hemoglobin H disease.

In various embodiments, the genome editing compositions are administered by direct injection to a cell, tissue, or organ of a subject in need of gene therapy, in vivo, e.g., bone marrow. In various other embodiments, cells are edited in vitro or ex vivo with reprogrammed nucleases contemplated herein, and optionally expanded ex vivo. The genome edited cells are then administered to a subject in need of therapy.

Preferred cells for use in the genome editing methods contemplated herein include autologous/autogeneic (“self”) cells, preferably hematopoietic cells, more preferably hematopoietic stem or progenitor cell, and even more preferably CD34⁺ cells.

As used herein, the terms “individual” and “subject” are often used interchangeably and refer to any animal that exhibits a symptom of a hemoglobinopathy that can be treated with the reprogrammed nucleases, genome editing compositions, gene therapy vectors, genome editing vectors, genome edited cells, and methods contemplated elsewhere herein.

Suitable subjects (e.g., patients) include laboratory animals (such as mouse, rat, rabbit, or guinea pig), farm animals, and domestic animals or pets (such as a cat or dog). Non-human primates and, preferably, human subjects, are included. Typical subjects include human patients that have, have been diagnosed with, or are at risk of having a hemoglobinopathy.

As used herein, the term “patient” refers to a subject that has been diagnosed with hemoglobinopathy that can be treated with the reprogrammed nucleases, genome editing compositions, gene therapy vectors, genome editing vectors, genome edited cells, and methods contemplated elsewhere herein.

As used herein “treatment” or “treating,” includes any beneficial or desirable effect on the symptoms or pathology of a hemoglobinopathy or hemoglobinopathic condition, and may include even minimal reductions in one or more measurable markers of the hemoglobinopathy or hemoglobinopathic condition. Treatment can optionally involve delaying of the progression of the hemoglobinopathy or hemoglobinopathic condition.

“Treatment” does not necessarily indicate complete eradication or cure of the hemoglobinopathy or hemoglobinopathic condition, or associated symptoms thereof.

As used herein, “prevent,” and similar words such as “prevention,” “prevented,” “preventing” etc., indicate an approach for preventing, inhibiting, or reducing the likelihood of the occurrence or recurrence of, hemoglobinopathy or hemoglobinopathic condition. It also refers to delaying the onset or recurrence of a hemoglobinopathy or hemoglobinopathic condition or delaying the occurrence or recurrence of the symptoms of hemoglobinopathy or hemoglobinopathic condition. As used herein, “prevention” and similar words also includes reducing the intensity, effect, symptoms and/or burden of a hemoglobinopathy or hemoglobinopathic condition prior to its onset or recurrence.

As used herein, the phrase “ameliorating at least one symptom of” refers to decreasing one or more symptoms of the hemoglobinopathy or hemoglobinopathic condition for which the subject is being treated, e.g., thalassemia, sickle cell disease, etc. In particular embodiments, the hemoglobinopathy or hemoglobinopathic condition being treated is β-thalassemia, wherein the one or more symptoms ameliorated include, but are not limited to, weakness, fatigue, pale appearance, jaundice, facial bone deformities, slow growth, abdominal swelling, dark urine, iron deficiency (in the absence of transfusion), requirement for frequent transfusions. In particular embodiments, the hemoglobinopathy or hemoglobinopathic condition being treated is sickle cell disease (SCD) wherein the one or more symptoms ameliorated include, but are not limited to, anemia; unexplained episodes of pain, such as pain in the abdomen, chest, bones or joints; swelling in the hands or feet; abdominal swelling; fever; frequent infections; pale skin or nail beds; jaundice; delayed growth; vision problems; signs or symptoms of stroke; iron deficiency (in the absence of transfusion), requirement for frequent transfusions.

As used herein, the term “amount” refers to “an amount effective” or “an effective amount” of a nuclease variant, genome editing composition, or genome edited cell sufficient to achieve a beneficial or desired prophylactic or therapeutic result, including clinical results.

A “prophylactically effective amount” refers to an amount of a nuclease variant, genome editing composition, or genome edited cell sufficient to achieve the desired prophylactic result. Typically but not necessarily, since a prophylactic dose is used in subjects prior to or at an earlier stage of disease, the prophylactically effective amount is less than the therapeutically effective amount.

A “therapeutically effective amount” of a nuclease variant, genome editing composition, or genome edited cell may vary according to factors such as the disease state, age, sex, and weight of the individual, and the ability to elicit a desired response in the individual. A therapeutically effective amount is also one in which any toxic or detrimental effects are outweighed by the therapeutically beneficial effects. The term “therapeutically effective amount” includes an amount that is effective to “treat” a subject (e.g., a patient). When a therapeutic amount is indicated, the precise amount of the compositions contemplated in particular embodiments, to be administered, can be determined by a physician in view of the specification and with consideration of individual differences in age, weight, tumor size, extent of infection or metastasis, and condition of the patient (subject).

The genome edited cells may be administered as part of a bone marrow or cord blood transplant in an individual that has or has not undergone bone marrow ablative therapy. In one embodiment, genome edited cells contemplated herein are administered in a bone marrow transplant to an individual that has undergone chemoablative or radioablative bone marrow therapy.

In one embodiment, a dose of genome edited cells is delivered to a subject intravenously. In preferred embodiments, genome edited hematopoietic stem cells are intravenously administered to a subject.

In one illustrative embodiment, the effective amount of genome edited cells provided to a subject is at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, or at least 10×10⁶ cells/kg, or more cells/kg, including all intervening doses of cells.

In another illustrative embodiment, the effective amount of genome edited cells provided to a subject is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, or about 10×10⁶ cells/kg, or more cells/kg, including all intervening doses of cells.

In another illustrative embodiment, the effective amount of genome edited cells provided to a subject is from about 2×10⁶ cells/kg to about 10×10⁶ cells/kg, about 3×10⁶ cells/kg to about 10×10⁶ cells/kg, about 4×10⁶ cells/kg to about 10×10⁶ cells/kg, about 5×10⁶ cells/kg to about 10×10⁶ cells/kg, 2×10⁶ cells/kg to about 6×10⁶ cells/kg, 2×10⁶ cells/kg to about 7×10⁶ cells/kg, 2×10⁶ cells/kg to about 8×10⁶ cells/kg, 3×10⁶ cells/kg to about 6×10⁶ cells/kg, 3×10⁶ cells/kg to about 7×10⁶ cells/kg, 3×10⁶ cells/kg to about 8×10⁶ cells/kg, 4×10⁶ cells/kg to about 6×10⁶ cells/kg, 4×10⁶ cells/kg to about 7×10⁶ cells/kg, 4×10⁶ cells/kg to about 8×10⁶ cells/kg, 5×10⁶ cells/kg to about 6×10⁶ cells/kg, 5×10⁶ cells/kg to about 7×10⁶ cells/kg, 5×10⁶ cells/kg to about 8×10⁶ cells/kg, or 6×10⁶ cells/kg to about 8×10⁶ cells/kg, including all intervening doses of cells.

Some variation in dosage will necessarily occur depending on the condition of the subject being treated. The person responsible for administration will, in any event, determine the appropriate dose for the individual subject.

In particular embodiments, a genome edited cell therapy is used to treat, prevent, or ameliorate a hemoglobinopathy, or condition associated therewith, comprising administering to subject having a β-globin genotype selected from the group consisting of: β^{E}/β⁰, β^{C}/β⁰, β⁰/β⁰, β^{E}/β^{E}, β^{C}/β⁺, β^{E}/β⁺, β⁰/β⁺, β⁺/β⁺, β^{C}/β^{C}, β^{E}/β^{S}, β⁰/β^{S}, β^{C}/β^{S}, β⁺/β^{S} or β^{S}/β^{S}, a therapeutically effective amount of the genome edited cells contemplated herein. In one embodiment, the genome edited cell therapy lacks functional BCL11A expression in erythroid cells, e.g., lacks the ability to sufficient BCL11A expression to repress or suppress γ-globin gene transcription and to transactivate 3-globin gene transcription. In one embodiment, the genome edited cells have a mutation introduced into a GATA-1 binding site in the BCL11A gene. In one embodiment, the genome edited cells have a mutation introduced into a consensus GATA-1 binding site (SEQ ID NO. 24) in the second intron of the BCL11A gene.

In particular embodiments, genome edited cell therapies contemplated herein are used to treat, prevent, or ameliorate a thalassemia, or condition associated therewith. Thalassemias treatable with the genome edited cell contemplated herein include, but are not limited to α-thalassemias and 3-thalassemias. In particular embodiments, a genome edited cell therapy is used to treat, prevent, or ameliorate a 3-thalassemia, or condition associated therewith, comprising administering to subject having a 3-globin genotype selected from the group consisting of: β^{E}/β⁰, β^{C}/β⁰, β⁰/β⁰, β^{C}/β^{C}, β^{E}/β^{E}, β^{E}/β⁺, β^{C}/β^{E}, β^{C}/β⁺, β⁰/β⁺, or β⁺/β⁺, a therapeutically effective amount of the genome edited cells contemplated herein. In one embodiment, the genome edited cell therapy lacks functional BCL11A expression in erythroid cells, e.g., lacks the ability to sufficient BCL11A expression to repress or suppress γ-globin gene transcription and to transactivate β-globin gene transcription. In one embodiment, the genome edited cells have a mutation introduced into a GATA-1 binding site in the BCL11A gene. In one embodiment, the genome edited cells have a mutation introduced into a consensus GATA-1 binding site (SEQ ID NO. 24) in the second intron of the BCL11A gene.

In particular embodiments, genome edited cell therapies contemplated herein are used to treat, prevent, or ameliorate a sickle cell disease or condition associated therewith. In particular embodiments, a genome edited cell therapy is used to treat, prevent, or ameliorate a sickle cell disease or condition associated therewith, comprising administering to subject having a β-globin genotype selected from the group consisting of: β^{E}/β^{S}, β⁰/β^{S}, β^{C}/β^{S}, β⁺/β^{S} or β^{S}/β^{S}, a therapeutically effective amount of the genome edited cells contemplated herein. In one embodiment, the genome edited cell therapy lacks functional BCL11A expression in erythroid cells, e.g., lacks the ability to sufficient BCL11A expression to repress or suppress γ-globin gene transcription and to transactivate β-globin gene transcription. In one embodiment, the genome edited cells have a mutation introduced into a GATA-1 binding site in the BCL11A gene. In one embodiment, the genome edited cells have a mutation introduced into a consensus GATA-1 binding site (SEQ ID NO. 24) in the second intron of the BCL11A gene.

In various embodiments, a subject is administered an amount of genome edited cells comprising a mutation into an erythroid specific enhancer in a BCL11A gene, effective to increase the expression of γ-globin in the subject. In particular embodiments, the amount of γ-globin gene expression in genome edited cells comprising a mutation into an erythroid specific enhancer in a BCL11A gene is increased at least about 10%, at least about 20%, at least about 30%, at least about 40%, at least about 50%, at least about 60%, at least about 70%, at least about 80%, at least about 90%, at least about 100%, at least about 2-fold, at least about 5-fold, at least about 10-fold, at least about 50-fold, at least about 100-fold, at least about 200-fold, at least about 300-fold, at least about 400-fold, at least about 500-fold, or at least about 1000-fold, or more compared to γ-globin gene expression in cells that have not undergone genome editing.

In various embodiments, a subject is administered an amount of genome edited cells comprising a mutation into an erythroid specific enhancer in a BCL11A gene, effective to increase the levels of HbF in the subject. In particular embodiments, the amount of HbF in genome edited cells comprising a mutation into an erythroid specific enhancer in a BCL11A gene is increased at least about 10%, at least about 20%, at least about 30%, at least about 40%, at least about 50%, at least about 60%, at least about 70%, at least about 80%, at least about 90%, at least about 100%, at least about 2-fold, at least about 5-fold, at least about 10-fold, at least about 50-fold, at least about 100-fold, at least about 200-fold, at least about 300-fold, at least about 400-fold, at least about 500-fold, or at least about 1000-fold, or more compared to the amount of HbF in cells that have not undergone genome editing.

One of ordinary skill in the art would be able to use routine methods in order to determine the appropriate route of administration and the correct dosage of an effective amount of a composition comprising genome edited cells contemplated herein. It would also be known to those having ordinary skill in the art to recognize that in certain therapies, multiple administrations of pharmaceutical compositions contemplated herein may be required to effect therapy.

One of the prime methods used to treat subjects amenable to treatment with genome edited hematopoietic stem and progenitor cell therapies is blood transfusion. Thus, one of the chief goals of the compositions and methods contemplated herein is to reduce the number of, or eliminate the need for, transfusions.

In particular embodiments, the drug product is administered once.

In certain embodiments, the drug product is administered 1, 2, 3, 4, 5, 6, 7, 8, 9, or 10 or more times over a span of 1 year, 2 years, 5, years, 10 years, or more.

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Identification of a Non-Canonical I-OnuI Homing Endonuclease Target Site in an Erythroid Enhancer in the Bcl11A Gene

The core GATA-1 motif (CTGnnnnnnnWGATAR; see SEQ ID NO: 24; FIG. 1) present in the BCL11A gene does not contain a canonical I-OnuI “central-4” cleavage motif: ATTC, TTTC, ATAC, ATAT, TTAC, and ATTT.

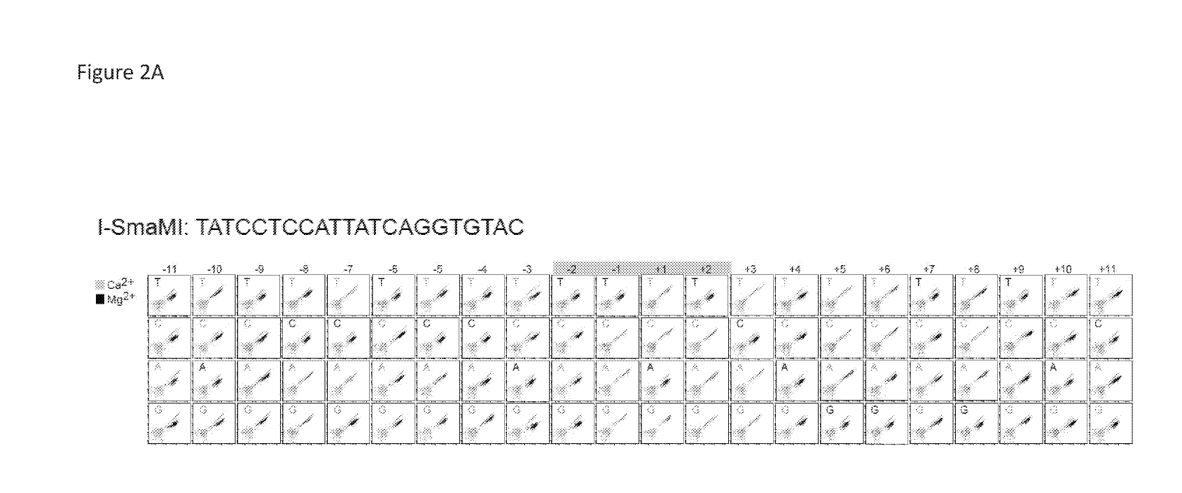

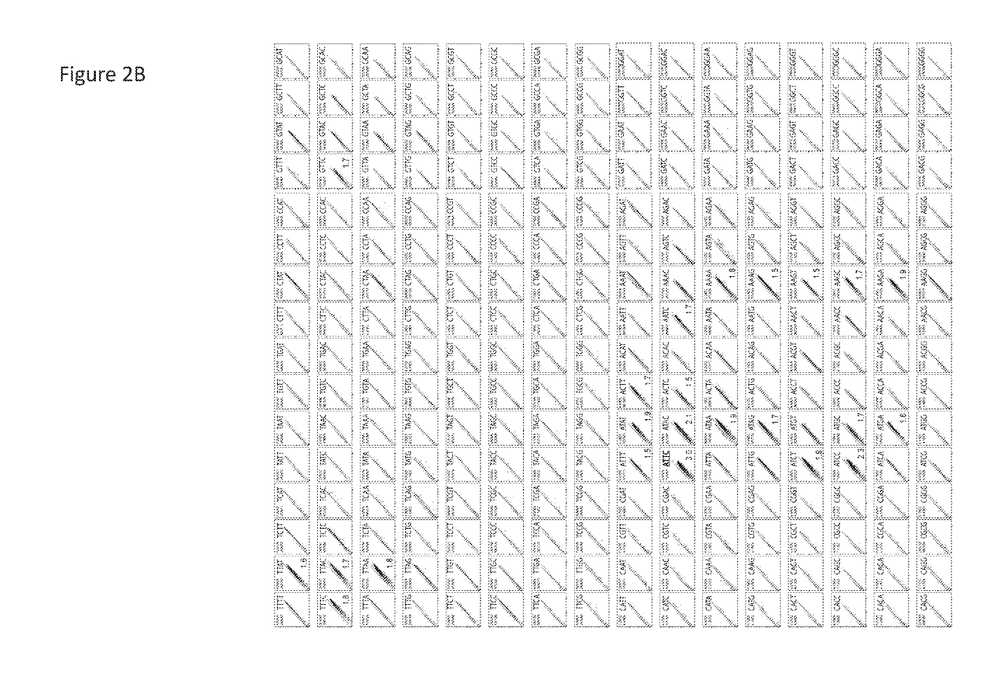

Surprisingly, the present inventors found that I-OnuI was a suitable starting scaffold for the development of a homing endonuclease variant or megaTAL targeting the GATA-1 motif. The target site “TTAT” (see SEQ ID NO: 25) was selected because its reverse complement “ATAA” is present in the core GATA-1 motif in the BCL11A gene (see SEQ ID NO: 24). Although not a canonical I-OnuI cleavage site, “TTAT” is the central-4 sequence (SEQ ID NO: 30) for the wild type I-SmaMI LHE (˜45% identity to I-OnuI). FIG. 2A.

In addition, the central-4 specificity of an I-OnuI variant HE that targets the CCR5 gene (SEQ ID NO: 31) was profiled using high throughput yeast surface display in vitro endonuclease assays (Jarjour, West-Foyle et al., 2009). A plasmid encoding the CCR5 targeting HE (SEQ ID NO: 32) was transformed into S. cerevisiae for surface display, then tested for cleavage activity against PCR-generated double-stranded DNA substrates comprising the CCR5 target site DNA sequence that contains each of the 256 possible central-4 sequences (SEQ ID NO: 33), including “TTAT”. The specificity profile showed that reprogrammed I-OnuI is able to cleave a target site comprising a non-canonical “TTAT” central-4 sequence. FIG. 2B.

I-OnuI was selected as the starting scaffold for the development of homing endonuclease variant or megaTAL targeting the GATA-1 motif in BCL11A.

### Example 2

### Reprogramming I-OnuI to Target the GATA-1 Motif in the Bcl11A Gene

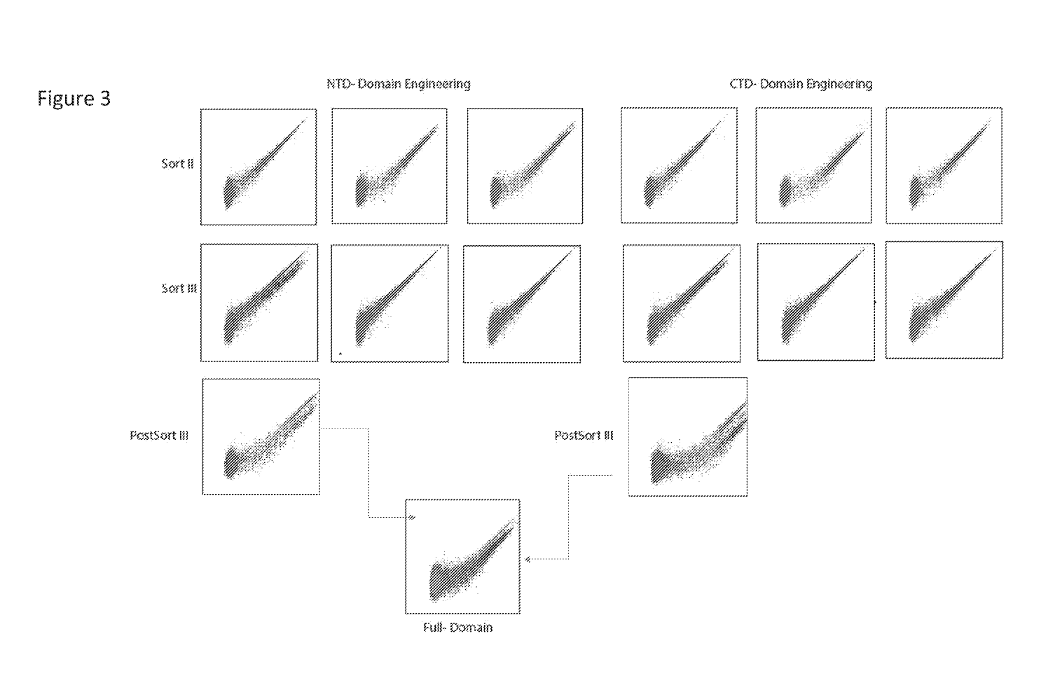

I-OnuI was reprogrammed to target the GATA-1 motif in the BCLL11A gene by constructing modular libraries containing variable amino acid residues in the DNA recognition interface. To construct the variants, degenerate codons were incorporated into I-OnuI DNA binding domains using oligonucleotides. The oligonucleotides encoding the degenerate codons were used as PCR templates to generate variant libraries by gap recombination in the yeast strain S. cerevisiae. Each variant library spanned either the N- or C-terminal I-OnuI DNA recognition domain and contained ˜10⁷ to 10⁸ unique transformants. The resulting surface display libraries were screened by flow cytometry for cleavage activity against target sites comprising the corresponding domains' “half-sites” (SEQ ID NOs: 28-29). FIG. 3.

Yeast displaying the N- and C-terminal domain reprogrammed I-OnuI HEs were purified and the plasmid DNA was extracted. PCR reactions were performed to amplify the reprogrammed domains, which were subsequently transformed into S. cerevisiae to create a library of reprogrammed domain combinations. Fully reprogrammed I-OnuI variants that recognize the complete target site (SEQ ID NO: 25) present in the GATA-1 motif in the BCL11A gene were identified from this library and purified.

### Example 3

### Reprogrammed I-OnuI Homing Endonucleases that Efficiently Target the GATA-1 Motif in the Bcl11A Gene

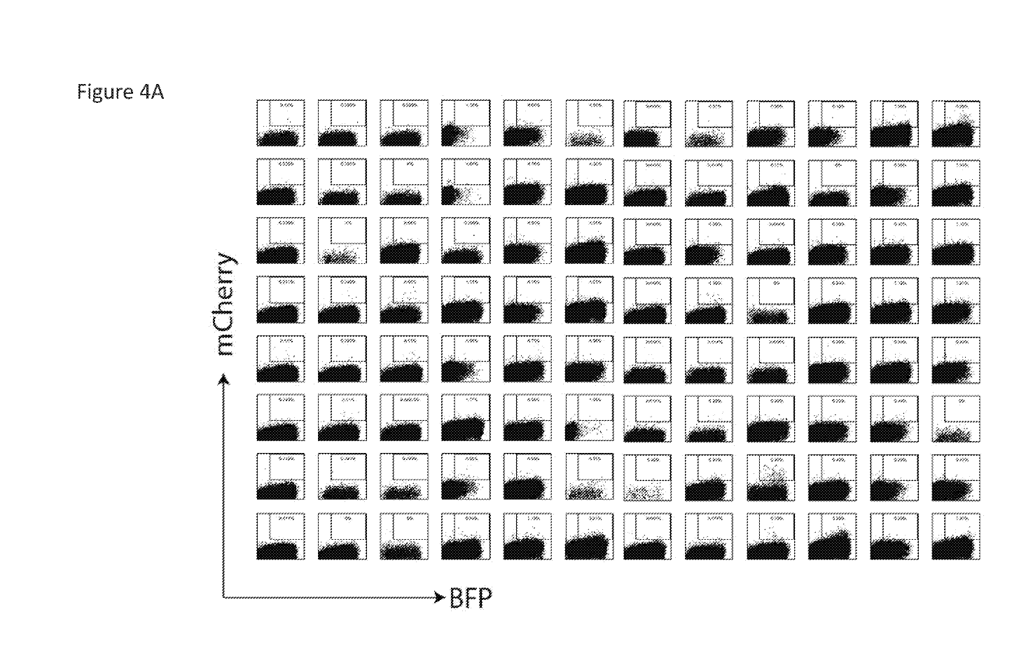

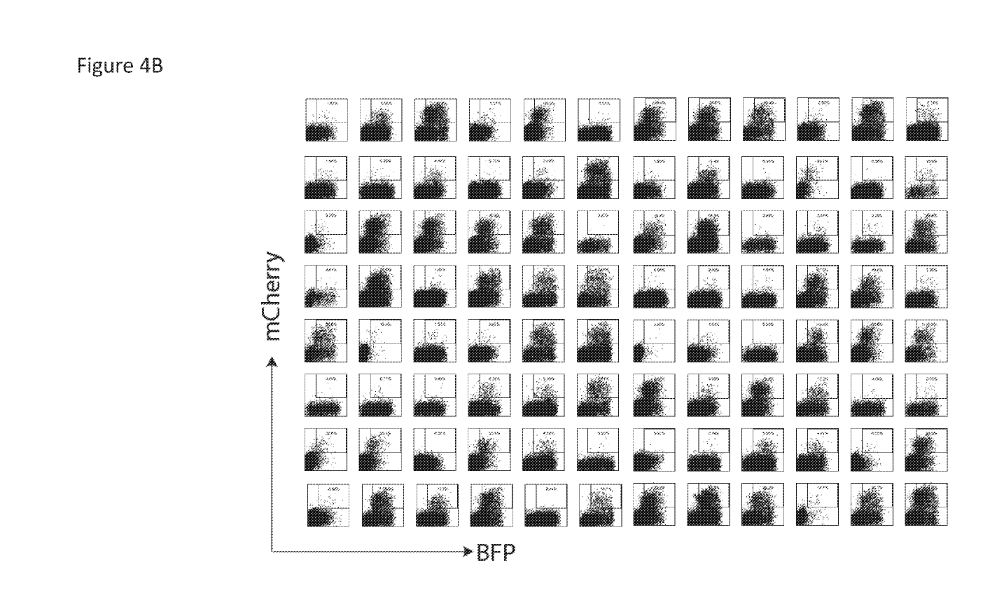

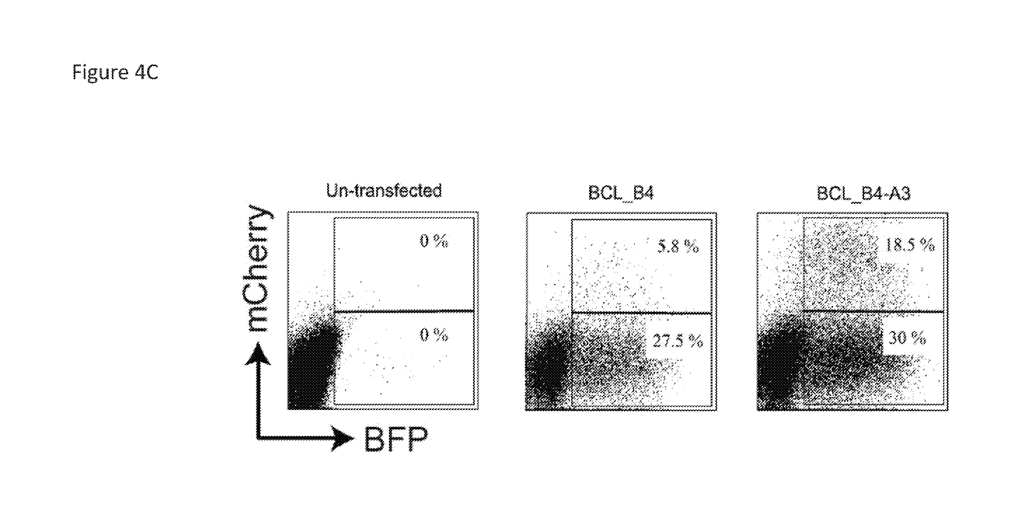

The activity of reprogrammed I-OnuI HEs that target the GATA-1 motif in the BCL11A gene was measured using a chromosomally integrated fluorescent reporter system (Certo et. al., 2011). Fully reprogrammed I-OnuI HEs that bind and cleave the BCL11A target sequence were cloned into mammalian expression plasmids and then individually transfected into a HEK 293T fibroblast cell line that was reprogrammed to contain the BCL11A target sequence upstream of an out-of-frame gene encoding the fluorescent mCherry protein. Cleavage of the embedded target site by the HE and the subsequent accumulation of small insertions or deletions, caused by DNA repair via the non-homologous end joining (NHEJ) pathway, results in approximately one out of three repaired loci placing the fluorescent reporter gene back “in-frame”. mCherry fluorescence is therefore a readout of endonuclease activity at the chromosomally embedded target sequence. The fully reprogrammed I-OnuI HEs that bind and cleave the BCL11A target site showed a moderate efficiency of mCherry expression in a cellular chromosomal context. FIG. 4A.

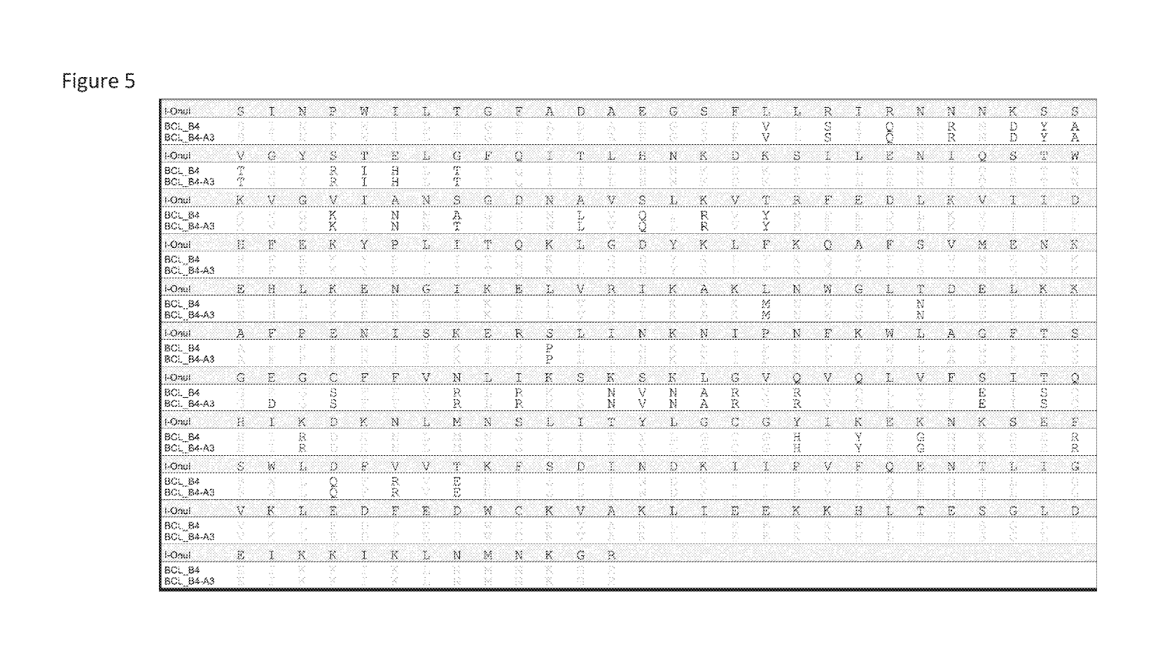

A secondary I-OnuI variant library was generated by performing random mutagenesis one of the reprogrammed I-OnuI HEs that targets the BCL11A target site, identified in the initial reporter screen (BCL11.A.B4, SEQ ID NO: 6). In addition, display-based flow sorting was performed under more stringent cleavage conditions (pH adjusted to 7.2) in an effort to isolate variants with improved catalytic efficiency. FIG. 4B. This process identified an I-OnuI variant, BCL11A.B4.A3 (SEQ ID NO: 7), which contain two amino acid mutations in the DNA recognition interface relative to the parental I-OnuI variant, and has an approximately 3-fold higher rate of mCherry expressing cells than the parental I-OnuI variant. FIG. 4C. FIG. 5 shows the relative alignments of representative I-OnuI as well as the positional information of the residues comprising the DNA recognition interface.

A tertiary I-OnuI variant library was generated by performing random mutagenesis one of the reprogrammed I-OnuI HEs that targets the BCL11A target site, identified in the secondary screen (BCL11A.B4.A3 (SEQ ID NO: 7). In addition, display-based flow sorting was performed under more stringent affinity conditions (50 pM) to isolate variants with improved binding characteristics. This process identified I-OnuI variants: BCL11A.B4.A3.C7 (SEQ ID NO: 8), BCL11A.B4.A3.E3 (SEQ ID NO: 9), BCL11A.B4.A3.B6 (SEQ ID NO: 10), BCL11A.B4.A3.H4 (SEQ ID NO: 11), BCL11A.B4.A3.B12 (SEQ ID NO: 12), BCL11A.B4.A3.A7 (SEQ ID NO: 13), BCL11A.B4.A3.C2 (SEQ ID NO: 14), BCL11A.B4.A3.G8 (SEQ ID NO: 15), BCL11A.B4.A3.A1 (SEQ ID NO: 16), BCL11A.B4.A3.A5 (SEQ ID NO: 17), BCL11A.B4.A3.B6.2 (SEQ ID NO: 18), and BCL11A.B4.A3.B7 (SEQ ID NO: 19).

### Example 4

### Affinity and Specificity of an Reprogrammed I-OnuI Homing Endonuclease that Efficiently Targets the GATA-1 Motif in the Bcl11A Gene

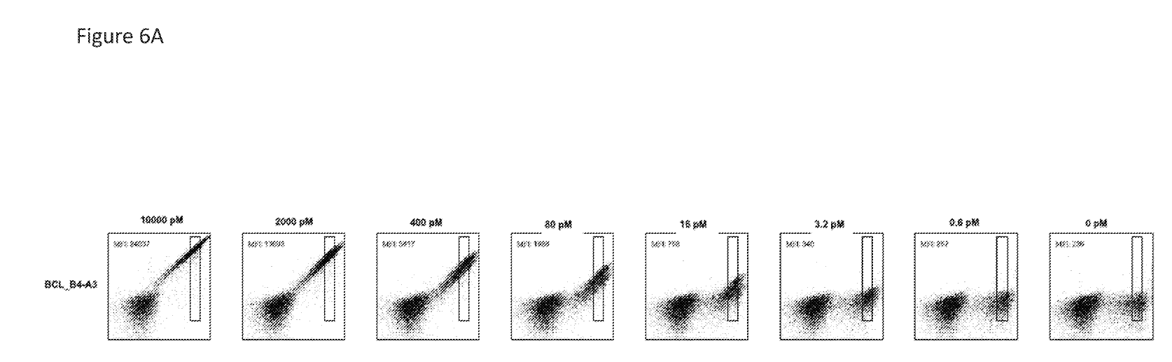

The DNA binding affinity and cleavage specificity of the I-OnuI variant BCL11A.B4.A3 was characterized. A plasmid encoding the BCL11A.B4.A3 variant identified during reprogramming (SEQ ID NO: 34) was transformed into S. cerevisiae for surface display. The affinity of I-OnuI variant BCL11A.B4.A3 was determined by equilibrium binding titrations, with an equilibrium dissociation constant estimated at ˜500 pM, which within range of several other wild type HEs in the I-OnuI sub-family (FIG. 6A).

Serial substitution analysis was used to determine cleavage specificity. Cleavage activity was assessed over a panel of DNA substrates where each target site position (SEQ ID NO: 25) was mutated to each of the 3 alternate base pairs. FIG. 6B. The CTD showed a higher degree of cleavage specificity than the NTD.

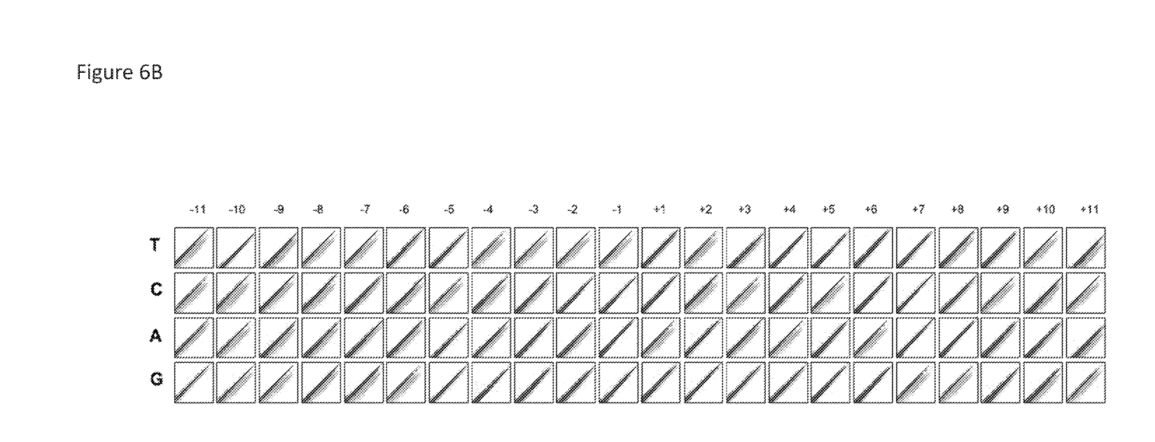

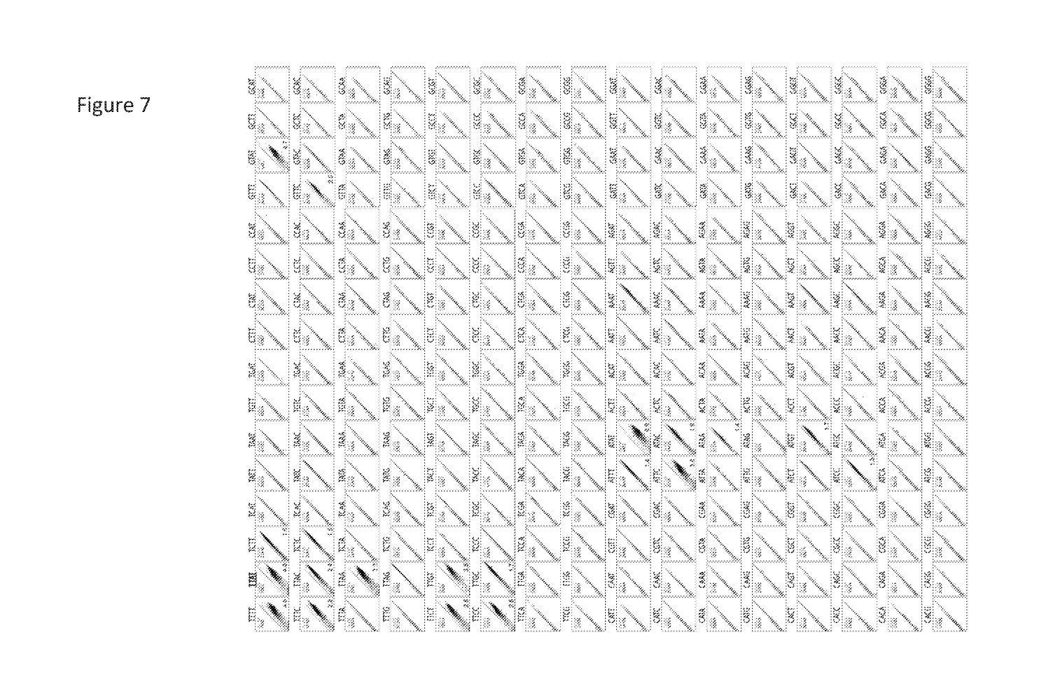

The target specificity of BCL11A.B4.A3 was also assessed because it is the first homing endonuclease reprogrammed to target a sequence that contains a non-natural central-4 sequence in its target site. DNA substrates comprising all 256 possible central-4 sequences within the BCL11A target site were generated (SEQ ID NO: 35). Each substrate was assayed against the I-OnuI variant BCL11A.B4.A3 displayed on the yeast surface (FIG. 7). Similar to the data presented in FIG. 2B, the I-OnuI variant BCL11A.B4.A3 showed a central-4 profile that included the TTAT motif, but that retained natural I-OnuI central-4 specificity.

### Example 5

### Efficient Disruption of the GATA-1 Motif in the Bcl11A Gene

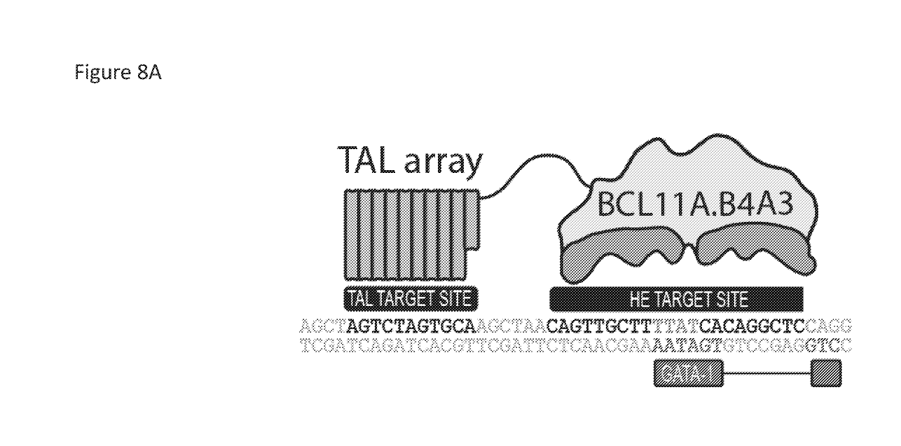

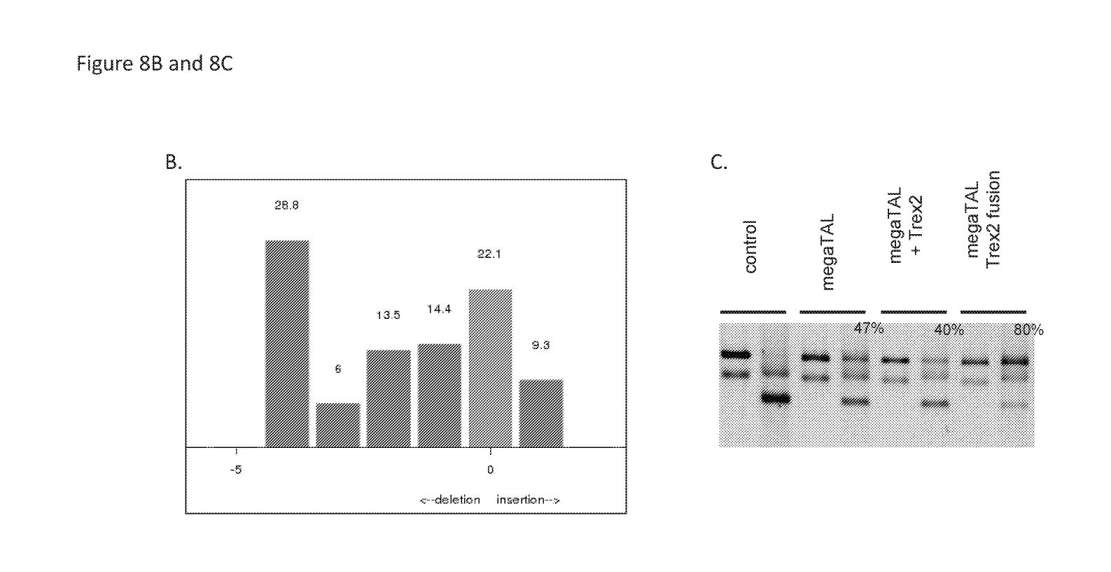

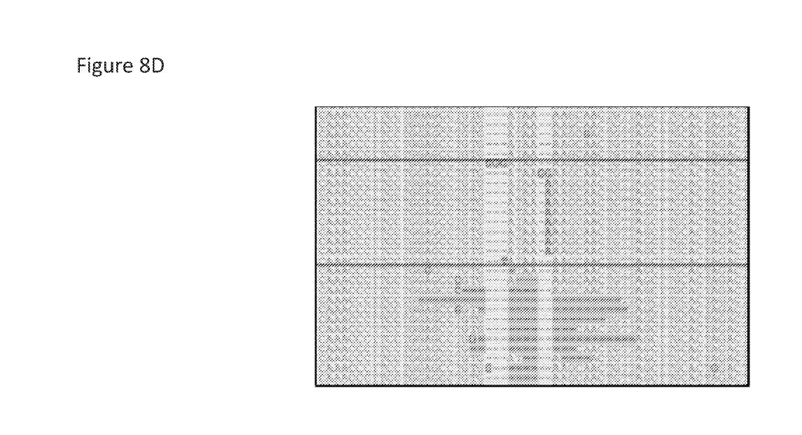

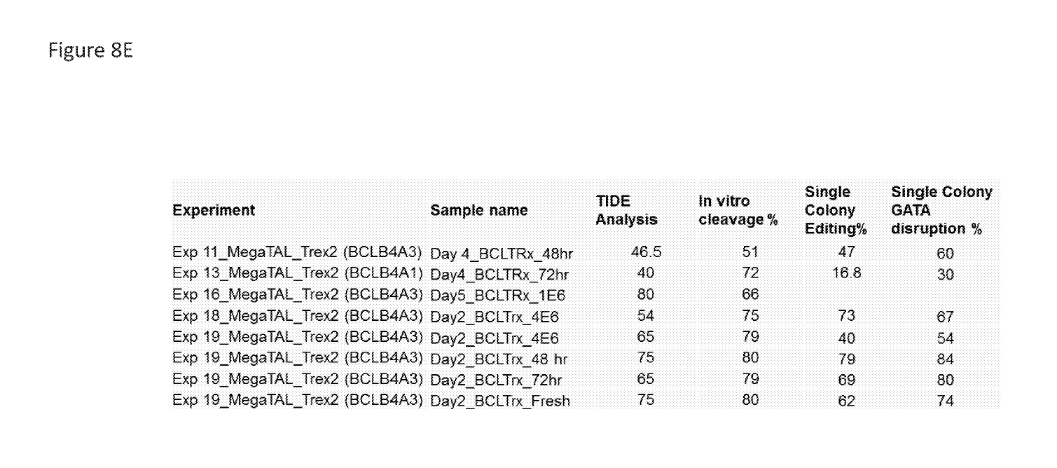

The I-OnuI variant BCL11A.B4.A3 was formatted as a megaTAL by appending an N-terminal 10.5 TAL array (eg. SEQ ID NOs: 21 and 36) corresponding to an 11 base pair TAL array target site upstream of the BCL11A target site (SEQ ID NO: 26), using methods described in Boissel et al., 2013. FIG. 8A. Another version of the megaTAL comprises a C-terminal fusion to Trex2 (e.g., SEQ ID NOs: 23 and 37).

The BCL11A megaTAL editing efficiency was assessed in primary human CD34+ cells by prestimulating the cells in cytokine-supplemented media for 48-72 hours, and then electroporating the cells with in vitro transcribed mRNA encoding the BCL11A megaTAL (e.g., SEQ ID NO: 36) and the megaTAL optionally formatted as a Trex2 fusion protein (e.g., SEQ ID NO: 37). Post-electroporation, cells were cultured for 1-4 days in cytokine-supplemented media, during which time aliquots were removed for genomic DNA isolation followed by PCR amplification across the BCL11A target site.

The frequency of small insertion/deletion (indel) events was measured using Tracking of Indels by DEcomposition (TIDE, see Brinkman et al., 2014), in vitro cleavage assays, and colony sequencing. FIG. 8B shows a representative TIDE analysis of amplicon indels and illustrates the predominance of +1, −1, −2, −3, or −4 indels at the target site of the BCL11A megaTAL. MegaTAL editing rates were confirmed by testing whether PCR amplicons spanning the BCL11A target site were capable of being re-cleaved by a recombinant BCL11A homing endonuclease. Treatment of cells with mRNA encoding the BCL11A megaTAL or BCL11A megaTAL-Trex2 fusion protein resulted in a significant fraction of amplicons that have been modified to the extent that they are no longer recognized and cleaved by the recombinant BCL11A megaTAL. FIG. 8C. The spectrum of indels was also characterized by cloning and sequencing PCR amplicons of individual colonies. The spectrum of indels at the BCL11A megaTAL target site is shown in FIG. 8D. FIG. 8E summarizes indel analyses over multiple experiments with different primary CD34+ donor cells, varied prestimulation windows, cell concentrations, and mRNA production batches.

The DNA sequencing studies demonstrate that the I-OnuI variant disrupted the GATA-1 consensus motif in a significant portion of treated cells. The editing efficiency of the BCL11A megaTAL was improved by fusion with Trex2.

### Example 6

### Efficient HDR at the GATA-1 Motif in the Bcl11A Gene

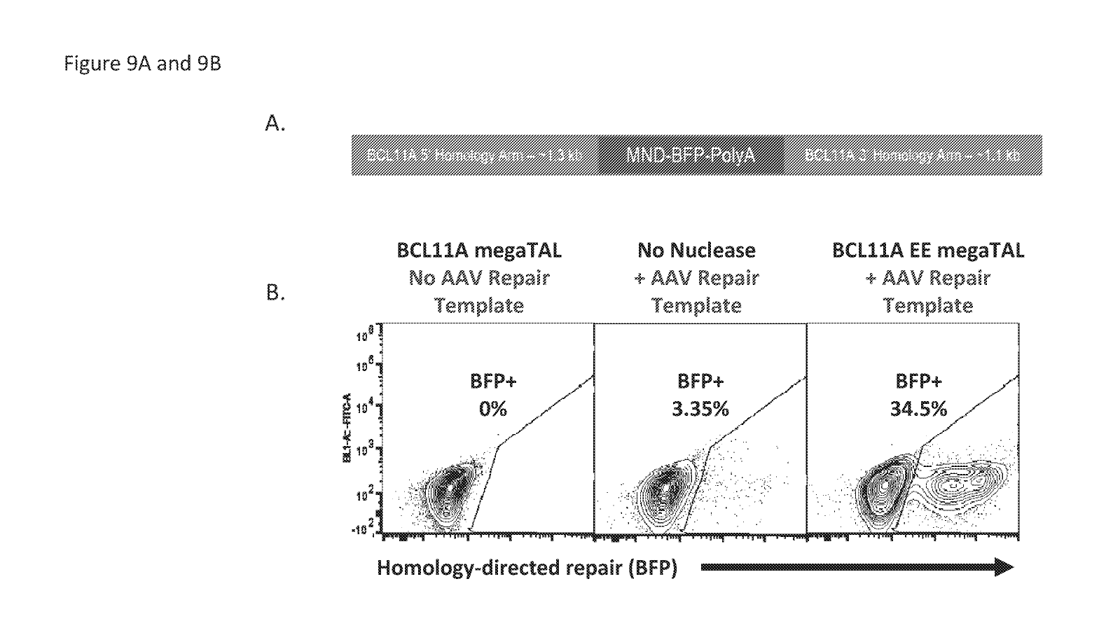

BCL11A megaTAL mRNA was electroporated into primary human CD34+ cells to assess homology directed repair of an AAV-delivered transgene at the GATA-1 target sequence in the BCL11A gene. An AAV2/6 vector comprising a constitutive promoter driving expression of BFP placed between sequences of DNA homology to the 5′ and 3′ regions flanking the BCL11A megaTAL target site was prepared using standard methods. FIG. 9A. Primary human CD34+ cells were prestimulated in cytokine-supplemented media then washed and electroporated in the presence or absence of mRNA encoding the BCL11A megaTAL (e.g., SEQ ID NO: 36). Cells were transduced with AAV either prior to electroporation or during a post-electroporation recovery step. Cells were cultured for 2-10 days in cytokine-supplemented media, during which time aliquots were removed for flow cytometry analysis of BFP expression to measure homology directed repair.

A substantial frequency of BFP+ cells were observed in the megaTAL plus AAV sample relative to the single agent control samples. FIG. 9B. The data show stable BFP expression from homology directed repair of the BCL11A target sequence with a BFP-containing transgene, as BFP expression from a transient episomal AAV genome disappears over a period of 2-4 days of culture following transduction.

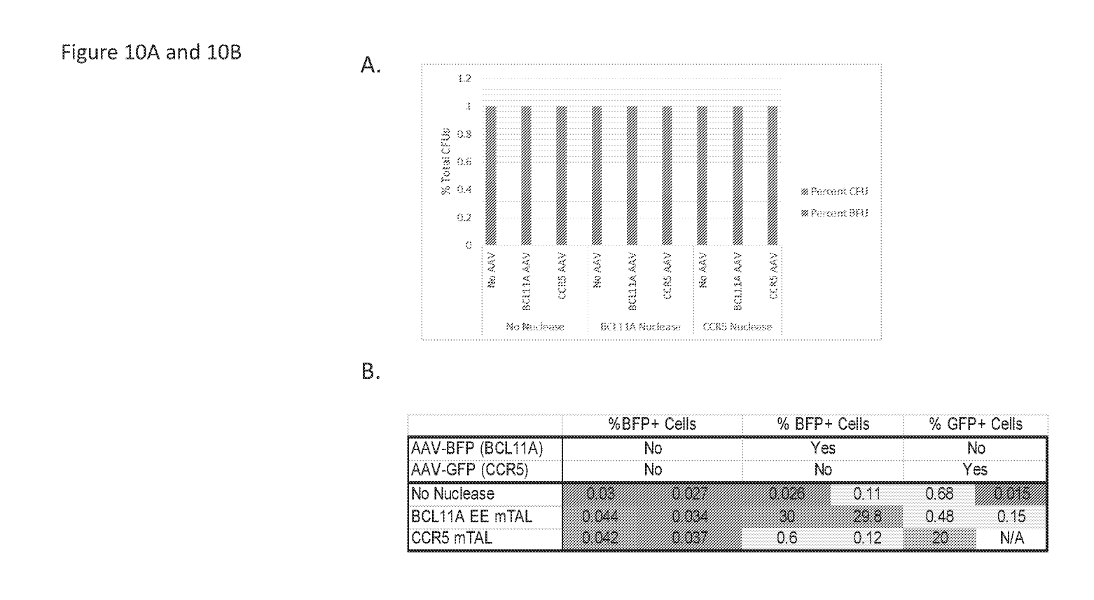

Methylcellulose assays were performed to determine whether megaTAL-based NHEJ or HDR altered the lineage characteristics of primary CD34+ cells. Primary human CD34+ cells were treated as described in the preceding paragraphs of this example, except that following a post-electroporation recovery step, cells were counted and plated into methylcellulose media for 14 days. After 14 days in culture, the colonies were scored for frequency and morphology. BCL11A megaTAL treated samples showed comparable mature colony phenotype frequency relative to control samples and did not show evidence of overt lineage skewing associated with genomic editing at the GATA-1 site in intron 2 of the BCL11A locus. FIG. 10A.

In addition, the BCL11A megaTAL plus AAV treated samples showed 30% and 29.8% BFP+ cells in duplicate cultures, while cells exposed to CCR5 megaTAL or no nuclease yielded <1% BFP+ cells. FIG. 10B. These results were consistent with significant homology directed repair mediated by BCL11A megaTAL in primitive hematopoietic stem and progenitor cells.

### Example 7

### CD34+ Cells Edited with a Bcl11A Targeting MegaTAL Upregulate HbF Levels

MegaTALs that efficiently disrupt the GATA-1 sequence in the BCL11A gene in primary human CD34+ cells increased HbF levels in the edited cells. Primary human CD34+ cells were prestimulated in cytokine-supplemented media, then washed and electroporated in the presence or absence of BCL11A megaTAL Trex2 fusion (e.g., SEQ ID NO: 37). After electroporation, cells were cultured for 5-7 days in an IMDM-based media containing serum, rhSCF, rhlL-3, and rhEPO, which promotes erythroid differentiation among cultured CD34+ cells. HbF levels were analyzed in differentiated erythroid cells by staining and flow cytometry using a directly conjugated anti-HbF antibody, or by HPLC analysis of globin chains.

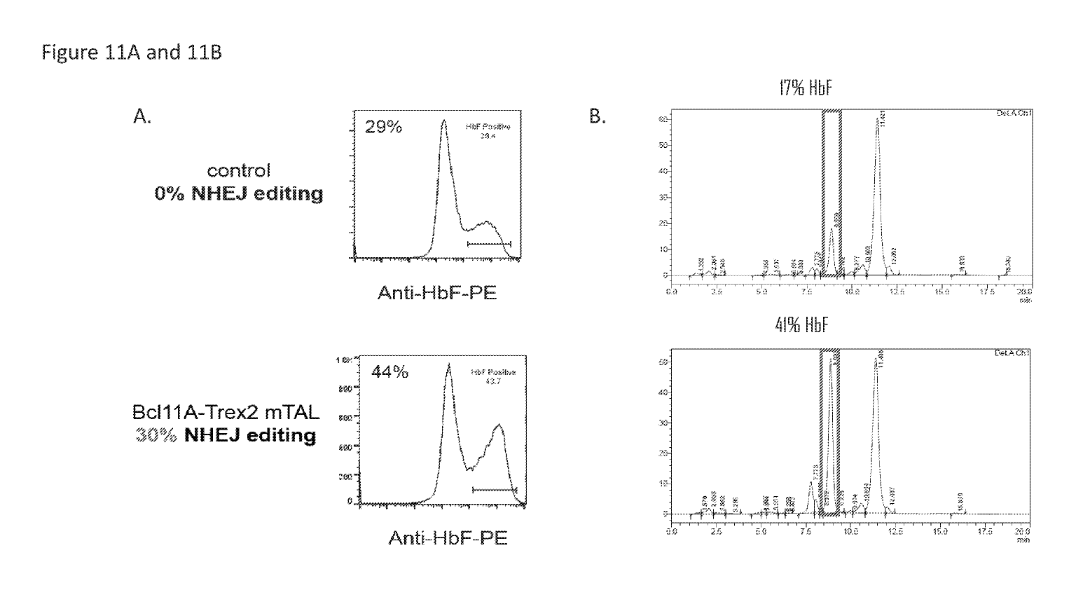

The frequency of HbF+ cells by flow cytometry increased in cells electroporated with mRNA encoding the BCL11A megaTAL-Trex2 fusion compared to control cultured cells. FIG. 11A. A substantial increase in HbF+ cells by HPLC was also observed in cells electroporated with mRNA encoding the BCL11A megaTAL-Trex2 fusion compared to control cultured cells. FIG. 11B. These data indicate that a BCL11A megaTAL targeting the GATA-1 site in the BCL11A gene derepressed γ-globin gene expression leading to an increase in the ratio of γ-globin to β-globin expression gene, thereby increasing HbF levels in the edited erythroid cells.

### Example 8

### Durable Genome Editing in Human Primary Long-Term NSG-Repopulating Cells in a Xenotransplantation Model

### Introduction

Human primary CD34+ cells were electroporated with megaTALs and transplanted into NSG mice to determine the durability of genome editing in long-term repopulating hematopoietic stem cells, which contribute to the long-term reconstitution of hematopoietic lineages following transplantation.

### Methods

Fresh human mobilized peripheral blood (mPB) CD34+ cells were prestimulated in a cytokine-containing media (SCF, TPO, FLT3-L) for 48 hours in a standard humidified tissue culture incubator (5% CO2). Following prestimulation, cells were harvested and enumerated. Cells were split into six groups of 25×10⁶ cells and resuspended in 400 μL of electroporation buffer. Cells were electroporated using a MaxCyte electroporation device and OC400 cuvettes with vehicle or with mRNA encoding BCL11A megaTAL, BCL11A megaTAL-Trex2, CCR5 megaTAL, and CCR5 megaTAL-Trex2 at a concentration of 100 μg/mL. Following electroporation, cells were transferred to flasks and diluted to 2×10⁶ cells/mL with a cytokine-containing media (SCF, TPO, FLT3-L, IL-3) and were incubated for approximately 20 hours at 30° C. The day following electroporation, the cells were cryopreserved prior to transplant.

Cells were thawed, washed, and split into two equal halves and resuspended in 2 mL SCGM+cytokines or an erythroid differentiation media and transferred to a standard 12-well non-adherent tissue culture plate. Cells cultured in SCGM+cytokines were maintained for up to an additional 6 days in a standard humidified tissue culture incubator (5% C02) and cells were enumerated over the course of the culture in order to establish growth curves. Additionally, after 5 days of culture, a subset of cells was collected for analysis of indel frequency, detailed below. Cells cultured in erythroid differentiation media were cultured for up to three weeks or until at least 30% of cells were Glycophorin A+ and CD71+, markers of erythroid differentiation. Once a sufficient level of erythroid differentiation was determined, cells were washed and resuspended in water and snap-frozen on dry ice. Extracted protein was then analyzed via ion-exchange high-performance liquid chromatography (IE-HPLC) for hemoglobin content.

Washed cells were resuspended in 200 μL SCGM and then transferred to 3 mL aliquots of cytokine-supplemented methylcellulose (for example, Methocult M4434 Classic). 1.1 mL was then transferred to parallel 35-mm tissue culture dishes using a blunt 16-gauge needle. Dishes were maintained in a standard humidified tissue culture incubator for 14-16 days and colonies were scored for size, morphology, and cellular composition.

Genomic DNA was extracted from cells and PCR amplification was performed to amplify the region of interest. Following a PCR clean-up, the amplicons were adapted for Miseq analysis and analyzed by targeted amplicon resequencing for insertion and deletion events.

To assess the impact of gene editing on human long-term hematopoietic stem cells, control and megaTAL-treated cells were thawed and washed prior to transplantation into the tail vein of sub-myeloablated adult NSG mice. Mice were housed in a pathogen-free environment per standard IACUC animal care guidelines. At 2 and 4 months post-transplant peripheral blood (PB) and bone marrow (BM), respectively, were harvested and analyzed for indel frequency, engraftment of human cells by staining with an anti-hCD45 antibody (BD #561864) followed by flow cytometry analysis, and HbF induction after erythroid differentiation.

In order to assess HbF induction with megaTAL treatment, BM is CD34+ enriched using Miltenyi small scale columns. CD34+ cells were then placed into an erythroid differentiation culture for up to three weeks or until at least 30% of cells were CD71+ and GPA+. Cells were then analyzed by IE-HPLC for hemoglobin content.

### Results

megaTAL Electroporation does not Affect CFC Formation

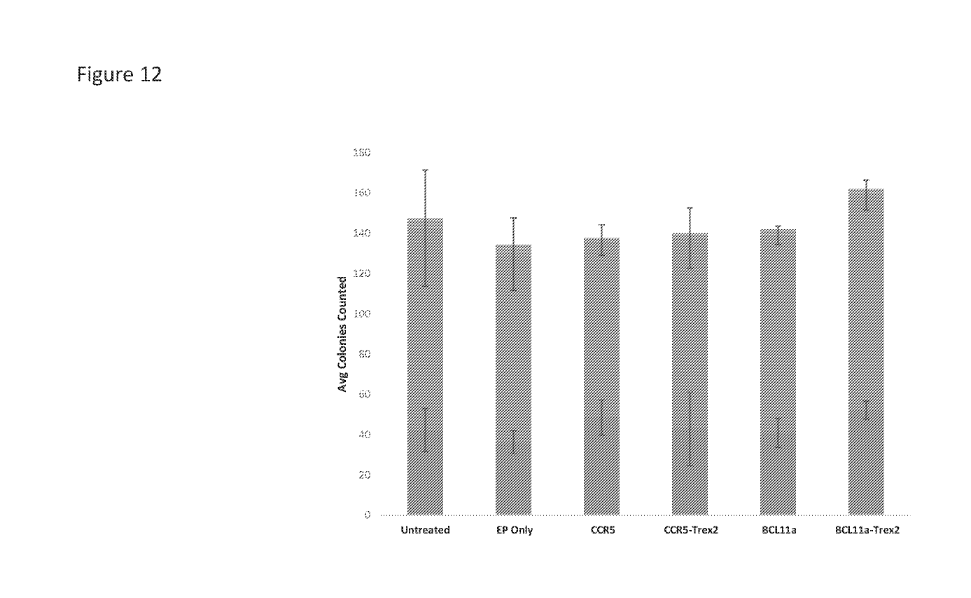

Cryopreserved control and megaTAL treated small-scale drug products were thawed and enumerated. 500 cells from each treatment group were transferred to MethoCult (H4434) and semi-solid cultures were initiated. After two weeks of culture, plates containing hematopoietic colonies were imaged using a STEMVision (Stemcell Technologies) and enumerated. Cells electroporated with megaTAL mRNA did not show differences in colony formation, the total number of colonies per group, or skewing of myeloid, erythroid, and stem cell-like phenotypes. FIG. 12.

megaTAL-Trex2 Fusion Proteins Increase Editing Rate

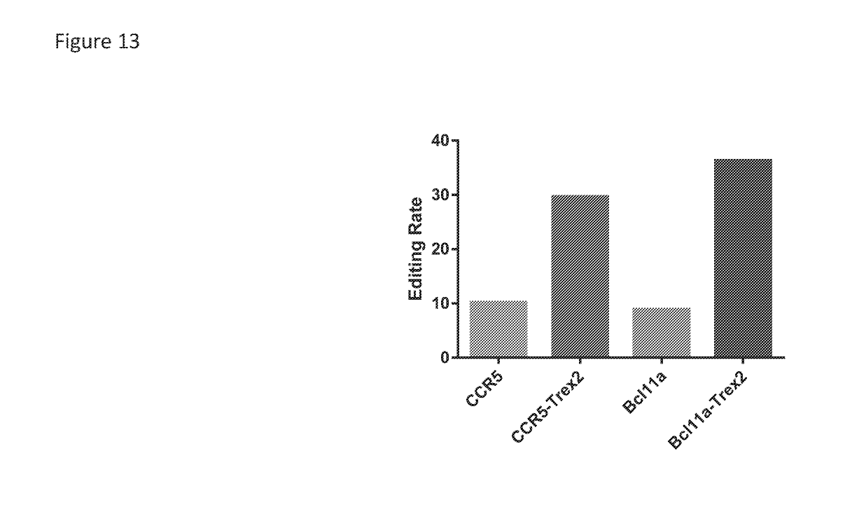

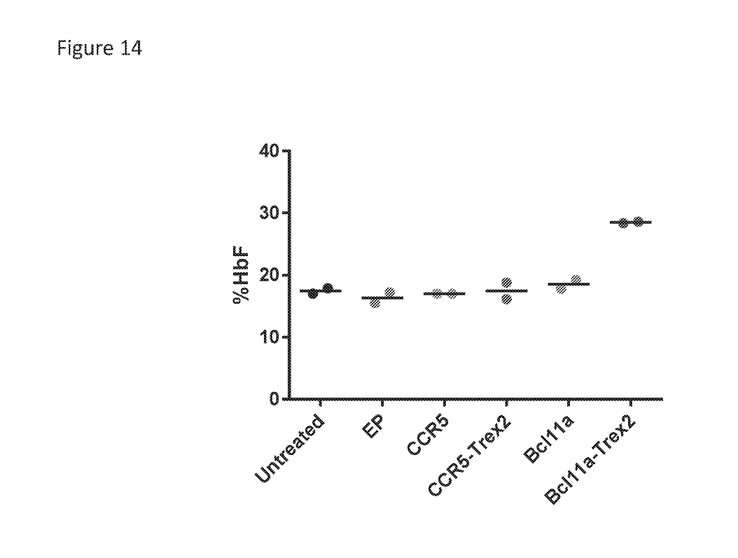

Cryopreserved control and megaTAL treated small-scale drug products were thawed and enumerated. Cells were then cultured for five days in cytokine-containing media prior to indel frequency analysis. Treatment of hCD34+ cells megaTALs directed against either CCR5 or BCL11A generated about 10% indels. CCR5 or BCL11A megaTAL-Trex2 fusion proteins increased the editing rate 2.9-fold and 4.1-fold respectively to approximately 30-35% indels. The background editing rates were less than 1%. FIG. 13.

BCL11A megaTAL-Trex2 Fusion Protein Induces Fetal Hemoglobin (HbF)

Cryopreserved control and megaTAL treated small-scale drug products were thawed, enumerated and placed into an erythroid differentiation culture. After ˜3 weeks of culture, markers of erythroid differentiation, cells were harvested, washed and lysed in water. Protein was analyzed by IE-HPLC for hemoglobin content. Background levels of HbF in this cell lot was ˜18%. Cells electroporated without mRNA or with mRNA encoding a CCR5 megaTAL, a CCR5 megaTAL-Trex2 megaTAL fusion protein, or a BCL11A megaTAL did not significantly alter HbF levels. However, cells electroporated with a BCL11A megaTAL-Trex2 fusion protein increased HbF 64% compared to untreated cells, to achieve ˜28% HbF.

Editing Frequency in Long-Term Repopulating Cells

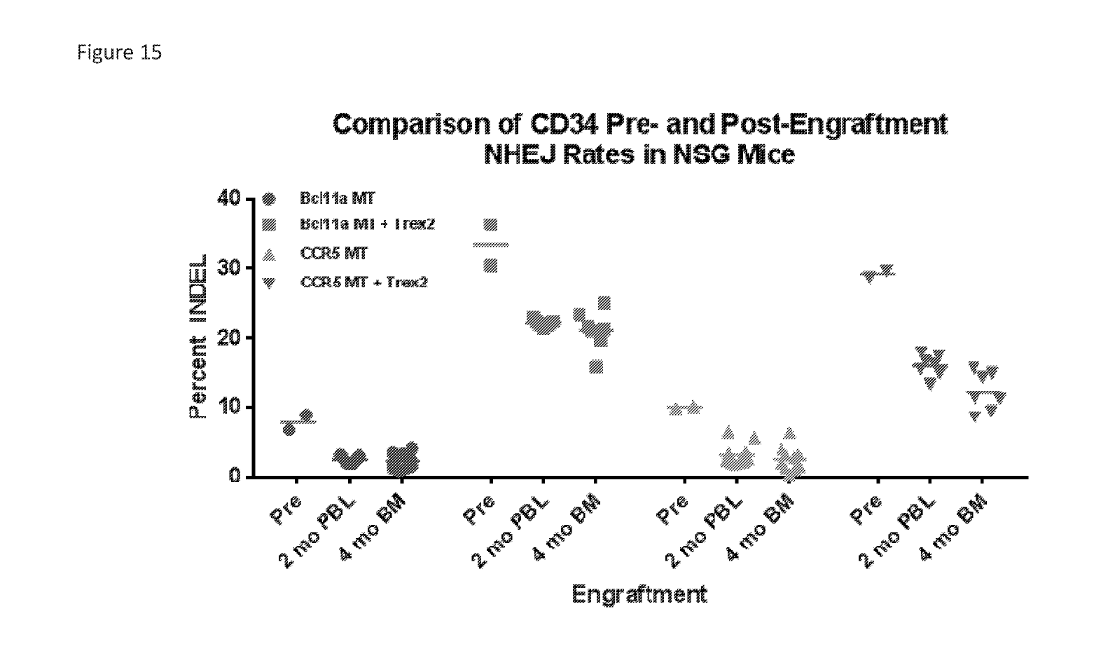

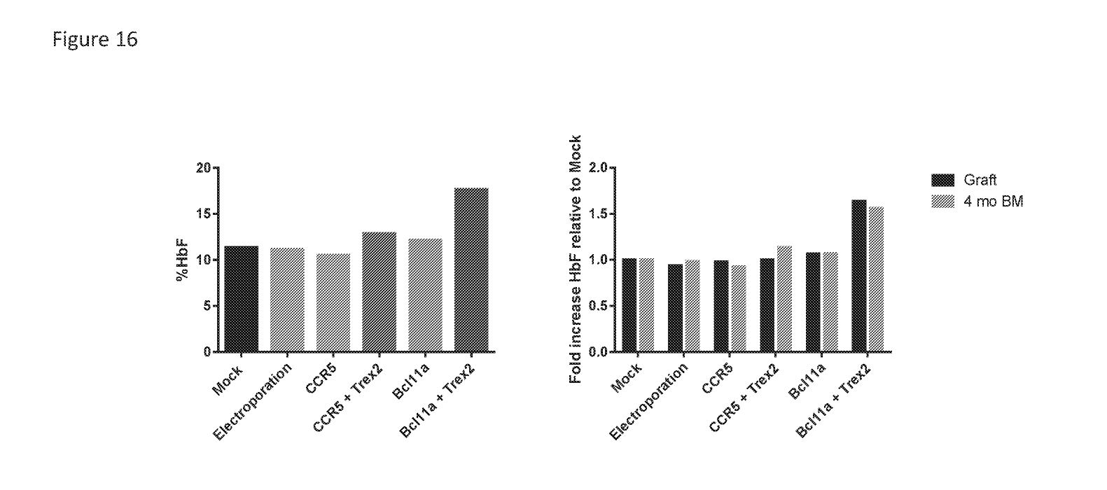

Editing rates, or the frequency of indels, were compared between the graft (Pre), a PB analysis at 2 months post-transplant (2 month PBL), and the 4 month BM editing analysis (4 month BM). PCR amplification was performed across the megaTAL target sites and the amplicons were sequenced using next generation sequencing. Genome editing rates remained above 20% at the 4-month time point in CD34+ cells electroporated with BCL11A-Trex2 megaTAL. FIG. 15.

BCL11A megaTAL-Trex2 Fusion Protein Increases HbF in Long-Term Repopulating Cells

Erythroid differentiated human CD34+ enriched cells coming from NSG BM were analyzed by IE-HPLC. The resulting HbF levels mirror those of the graft. The background HbF level in these cultures was approximately 11%. Cells electroporated without mRNA or with mRNA encoding a CCR5 megaTAL, a CCR5 megaTAL-Trex2 megaTAL fusion protein, or a BCL11A megaTAL did not significantly alter HbF levels. However, treatment with a BCL11A-Trex2 megaTAL increased HbF production ˜18%. This is a >50% increase over control cells.

### CONCLUSION

BCL11A megaTALs generate high genome editing rates consistent with durable genomic editing of the long-term repopulating hematopoietic stem cell population within the edited CD34+ population of transplanted cells.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A polypeptide comprising a homing endonuclease (HE) variant that cleaves a target site in the human B-cell lymphoma/leukemia 11A (BCL11A) gene.

2. The polypeptide of claim 1, wherein the HE variant is an LAGLIDADG homing endonuclease (LHE) variant.

3. The polypeptide of claim 1, or claim 2, wherein the polypeptide comprises a biologically active fragment of the HE variant.

4. The polypeptide of claim 3, wherein the biologically active fragment lacks the 1, 2, 3, 4, 5, 6, 7, or 8 N-terminal amino acids compared to a corresponding wild type HE.

5. The polypeptide of claim 4, wherein the biologically active fragment lacks the 4 N-terminal amino acids compared to a corresponding wild type HE.

6. The polypeptide of claim 4, wherein the biologically active fragment lacks the 8 N-terminal amino acids compared to a corresponding wild type HE.

7. The polypeptide of claim 3, wherein the biologically active fragment lacks the 1, 2, 3, 4, or 5 C-terminal amino acids compared to a corresponding wild type HE.

8. The polypeptide of claim 7, wherein the biologically active fragment lacks the C-terminal amino acid compared to a corresponding wild type HE.

9. The polypeptide of claim 7, wherein the biologically active fragment lacks the 2 C-terminal amino acids compared to a corresponding wild type HE.

10. The polypeptide of any one of claims 1 to 9, wherein the HE variant is a variant of an LHE selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, and I-Vdil41I.

11. The polypeptide of any one of claims 1 to 10, wherein the HE variant is a variant of an LHE selected from the group consisting of: I-CpaMI, I-HjeMI, I-OnuI, I-PanMI, and SmaMI.

12. The polypeptide of any one of claims 1 to 11, wherein the HE variant is an I-OnuI LHE variant.

13. The polypeptide of any one of claims 1 to 12, wherein the HE variant comprises one or more amino acid substitutions at amino acid positions selected from the group consisting of: 19, 24, 26, 28, 30, 32, 34, 35, 36, 37, 38, 40, 42, 44, 46, 48, 68, 70, 72, 75, 76, 77, 78, 80, 82, 168, 180, 182, 184, 186, 188, 189, 190, 191, 192, 193, 195, 197, 199, 201, 203, 223, 225, 227, 229, 231, 232, 234, 236, 238, and 240 of an I-OnuI LHE amino acid sequence as set forth in SEQ ID NOs: 1-5, or a biologically active fragment thereof.

14. The polypeptide of any one of claims 1 to 13, wherein the HE variant comprises at least 5, at least 15, preferably at least 25, more preferably at least 35, or even more preferably at least 40 or more amino acid substitutions at amino acid positions selected from the group consisting of: 19, 24, 26, 28, 30, 32, 34, 35, 36, 37, 38, 40, 42, 44, 46, 48, 68, 70, 72, 75, 76, 77, 78, 80, 82, 168, 180, 182, 184, 186, 188, 189, 190, 191, 192, 193, 195, 197, 199, 201, 203, 223, 225, 227, 229, 231, 232, 234, 236, 238, and 240 of an I-OnuI LHE amino acid sequence as set forth in SEQ ID NOs: 1-5, or a biologically active fragment thereof.

15. The polypeptide of any one of claims 1 to 12, wherein the HE variant comprises at least 5, at least 15, preferably at least 25, more preferably at least 35, or even more preferably at least 40 or more amino acid substitutions at amino acid positions selected from the group consisting of: 26, 28, 30, 32, 34, 35, 36, 37, 40, 41, 42, 44, 48, 50, 53, 68, 70, 72, 76, 78, 80, 82, 138, 143, 159, 178, 180, 184, 186, 189, 190, 191, 192, 193, 195, 201, 203, 207, 223, 225, 227, 232, 236, 238, and 240 of an I-OnuI LHE amino acid sequence as set forth in SEQ ID NOs: 1-19, or a biologically active fragment thereof.

16. The polypeptide of any one of claims 1 to 15, wherein the HE variant comprises at least 5, at least 15, preferably at least 25, more preferably at least 35, or even more preferably at least 40 or more of the following amino acid substitutions: L26V, L26R, L26Y, R28S, R28G, R30Q, R30H, N32R, N32S, N32K, N33S, K34D, K34N, S35Y, S36A, V37T, S40R, T41I, E42H, E42R, G44T, G44R, T48I, T48G, T48V, H50R, D53E, V68K, V68R, A70N, A70E, A70N, A70Q, A70L, A70S, S72A, S72T, S72V, S72M, A76L, A76H, A76R, S78Q, K80R, K80V, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

17. The polypeptide of any one of claims 1 to 16, wherein the HE variant comprises the following amino acid substitutions: L26V, R28S, R30Q, N32R, K34D, S35Y, S36A, V37T, S40R, T41I, E42H, G44T, V68K, A70N, S72A, A76L, S78Q, K80R, T82Y, L138M, T143N, S159P, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

18. The polypeptide of any one of claims 1 to 16, wherein the HE variant comprises the following amino acid substitutions: L26V, R28S, R30Q, N32R, K34D, S35Y, S36A, V37T, S40R, T41I, E42H, G44T, V68K, A70N, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

19. The polypeptide of any one of claims 1 to 16, wherein the HE variant comprises the following amino acid substitutions: L26V, R30Q, N32S, K34D, S35Y, S36A, V37T, S40R, T41I, E42H, G44T, V68K, A70N, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

20. The polypeptide of any one of claims 1 to 16, wherein the HE variant comprises the following amino acid substitutions: L26V, R28S, R30Q, N32K, K34N, S35Y, S36A, V37T, S40R, T41I, E42H, G44T, T48I, V68K, A70N, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

21. The polypeptide of any one of claims 1 to 16, wherein the HE variant comprises the following amino acid substitutions: L26V, R28S, R30Q, N32R, K34D, S35Y, S36A, V37T, S40R, T41I, E42R, G44T, T48I, V68K, A70N, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

22. The polypeptide of any one of claims 1 to 16, wherein the HE variant comprises the following amino acid substitutions: L26V, R28G, R30Q, N32R, K34D, S35Y, S36A, V37T, S40R, T41I, E42R, G44T, H50R, V68K, A70N, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

23. The polypeptide of any one of claims 1 to 16, wherein the HE variant comprises the following amino acid substitutions: L26V, R28S, R30H, N32R, K34D, S35Y, S36A, V37T, S40R, T41I, E42H, G44R, V68K, A70N, S72T, A76H, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

24. The polypeptide of any one of claims 1 to 16, wherein the HE variant comprises the following amino acid substitutions: L26R, R28S, R30Q, N32R, K34D, S35Y, S36A, V37T, S40R, T41I, E42H, G44R, V68K, A70N, S72TA76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

25. The polypeptide of any one of claims 1 to 16, wherein the HE variant comprises the following amino acid substitutions: L26Y, R28S, R30Q, N32R, K34D, S35Y, S36A, V37T, S40R, T41I, E42H, G44R, D53E, V68R, A70E, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

26. The polypeptide of any one of claims 1 to 16, wherein the HE variant comprises the following amino acid substitutions: L26V, R28S, R30Q, N32R, N33S, K34D, S35Y, S36A, V37T, S40R, T41I, E42H, G44R, D53E, V68K, A70N, S72T, A76L, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

27. The polypeptide of any one of claims 1 to 16, wherein the HE variant comprises the following amino acid substitutions: L26V, R28S, R30Q, N32R, N33S, K34D, S35Y, S36A, V37T, S40R, T41I, E42H, G44R, T48G, V68K, S72V, A76R, S78Q, K80V, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

28. The polypeptide of any one of claims 1 to 16, wherein the HE variant comprises the following amino acid substitutions: L26V, R28S, R30Q, N32R, N33S, K34D, S35Y, S36A, V37T, S40R, T41I, E42H, G44R, T48G, V68K, A70Q, S72M, A76R, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

29. The polypeptide of any one of claims 1 to 16, wherein the HE variant comprises the following amino acid substitutions: L26V, R28S, R30Q, N32R, N33S, K34D, S35Y, S36A, V37T, S40R, T41I, E42H, G44R, T48G, V68K, A70L, S72V, A76H, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

30. The polypeptide of any one of claims 1 to 16, wherein the HE variant comprises the following amino acid substitutions: L26V, R28S, R30Q, N32R, N33S, K34D, S35Y, S36A, V37T, S40R, T41I, E42H, G44R, T48V, V68K, A70S, S72V, A76H, S78Q, K80R, T82Y, L138M, T143N, S159P, E178D, C180S, N184R, I186R, K189N, S190V, K191N, L192A, G193R, Q195R, S201E, T203S, K207R, Y223H, K225Y, K227G, F232R, D236Q, V238R, and T240E, in reference to an I-OnuI LHE amino acid sequence as set forth in SEQ ID NOs: 1-5, or a biologically active fragment thereof.

31. The polypeptide of any one of claims 1 to 30, wherein the HE variant comprises an amino acid sequence that is at least 80%, preferably at least 85%, more preferably at least 90%, or even more preferably at least 95% identical to the amino acid sequence set forth in any one of SEQ ID NOs: 6-19, or a biologically active fragment thereof.

32. The polypeptide of any one of claims 1 to 31, wherein the HE variant comprises the amino acid sequence set forth in SEQ ID NO: 6, or a biologically active fragment thereof.

33. The polypeptide of any one of claims 1 to 31 wherein the HE variant comprises the amino acid sequence set forth in SEQ ID NO: 7, or a biologically active fragment thereof.

34. The polypeptide of any one of claims 1 to 31 wherein the HE variant comprises the amino acid sequence set forth in SEQ ID NO: 8, or a biologically active fragment thereof.

35. The polypeptide of any one of claims 1 to 31 wherein the HE variant comprises the amino acid sequence set forth in SEQ ID NO: 9, or a biologically active fragment thereof.

36. The polypeptide of any one of claims 1 to 31 wherein the HE variant comprises the amino acid sequence set forth in SEQ ID NO: 10, or a biologically active fragment thereof.

37. The polypeptide of any one of claims 1 to 31 wherein the HE variant comprises the amino acid sequence set forth in SEQ ID NO: 11, or a biologically active fragment thereof.

38. The polypeptide of any one of claims 1 to 31 wherein the HE variant comprises the amino acid sequence set forth in SEQ ID NO: 12, or a biologically active fragment thereof.

39. The polypeptide of any one of claims 1 to 31 wherein the HE variant comprises the amino acid sequence set forth in SEQ ID NO: 13, or a biologically active fragment thereof.

40. The polypeptide of any one of claims 1 to 31 wherein the HE variant comprises the amino acid sequence set forth in SEQ ID NO: 14, or a biologically active fragment thereof.

41. The polypeptide of any one of claims 1 to 31 wherein the HE variant comprises the amino acid sequence set forth in SEQ ID NO: 15, or a biologically active fragment thereof.

42. The polypeptide of any one of claims 1 to 31 wherein the HE variant comprises the amino acid sequence set forth in SEQ ID NO: 16, or a biologically active fragment thereof.

43. The polypeptide of any one of claims 1 to 31 wherein the HE variant comprises the amino acid sequence set forth in SEQ ID NO: 17, or a biologically active fragment thereof.

44. The polypeptide of any one of claims 1 to 31 wherein the HE variant comprises the amino acid sequence set forth in SEQ ID NO: 18, or a biologically active fragment thereof.

45. The polypeptide of any one of claims 1 to 31 wherein the HE variant comprises the amino acid sequence set forth in SEQ ID NO: 19, or a biologically active fragment thereof.

46. The polypeptide of any one of claims 1-45, further comprising a DNA binding domain.

47. The polypeptide of claim 46, wherein the DNA binding domain is selected from the group consisting of: a TALE DNA binding domain and a zinc finger DNA binding domain.

48. The polypeptide of claim 47, wherein the TALE DNA binding domain comprises about 9.5 TALE repeat units to about 15.5 TALE repeat units.

49. The polypeptide of claim 47 or claim 48, wherein the TALE DNA binding domain binds a polynucleotide sequence in the BCL11A gene.

50. The polypeptide of any one of claims 47 to 48, wherein the TALE DNA binding domain binds the polynucleotide sequence set forth in SEQ ID NO: 26.

51. The polypeptide of claim 47, wherein the zinc finger DNA binding domain comprises 2, 3, 4, 5, 6, 7, or 8 zinc finger motifs.

52. The polypeptide of any one of claims 1 to 51, further comprising a peptide linker and an end-processing enzyme or biologically active fragment thereof.

53. The polypeptide of any one of claims 1 to 52, further comprising a viral self-cleaving 2A peptide and an end-processing enzyme or biologically active fragment thereof.

54. The polypeptide of claim 52 or claim 53, wherein the end-processing enzyme or biologically active fragment thereof has 5′-3′ exonuclease, 5′-3′ alkaline exonuclease, 3′-5′ exonuclease, 5′ flap endonuclease, helicase, template-dependent DNA polymerase or template-independent DNA polymerase activity.

55. The polypeptide of any one of claims 52 to 54, wherein the end-processing enzyme comprises Trex2 or a biologically active fragment thereof.

56. The polypeptide of any one of claims 1 to 55, wherein the polypeptide cleaves the human BCL11A gene at the polynucleotide sequence set forth in SEQ ID NO: 25 or SEQ ID NO: 27.

57. A polynucleotide encoding the polypeptide of any one of claims 1 to 56.

58. An mRNA encoding the polypeptide of any one of claims 1 to 56.

59. A cDNA encoding the polypeptide of any one of claims 1 to 56.

60. A vector comprising a polynucleotide encoding the polypeptide of any one of claims 1 to 56.

61. A cell comprising the polypeptide of any one of claims 1 to 56.

62. A cell comprising a polynucleotide encoding the polypeptide of any one of claims 1 to 56.

63. A cell comprising the vector of claim 60.

64. A cell comprising one or more genome modifications introduced by the polypeptide of any one of claims 1 to 56.

65. The cell of any one of claims 61 to 64, wherein the cell is a hematopoietic cell.

66. The cell of any one of claims 61 to 65, wherein the cell is a hematopoietic stem or progenitor cell.

67. The cell of any one of claims 61 to 66, wherein the cell is a CD34⁺ cell.

68. The cell of any one of claims 61 to 67, wherein the cell is a CD133⁺ cell.

69. A composition comprising a cell according to any one of claims 61 to 68.

70. A composition comprising the cell according to any one of claims 61 to 68 and a physiologically acceptable carrier.

71. A method of editing a BCL11A gene in a population of cells comprising: introducing a polynucleotide encoding the polypeptide of any one of claims 1 to 56 into the cell, wherein expression of the polypeptide creates a double strand break at a target site in a BCL11A gene.

72. A method of editing a BCL11A gene in a population of cells comprising: introducing a polynucleotide encoding the polypeptide of any one of claims 1 to 56 into the cell, wherein expression of the polypeptide creates a double strand break at a target site in a BCL11A gene, wherein the break is repaired by non-homologous end joining (NHEJ).

73. A method of editing a BCL11A gene in a population of cells comprising: introducing a polynucleotide encoding the polypeptide of any one of claims 1 to 56 and a donor repair template into the cell, wherein expression of the polypeptide creates a double strand break at a target site in a BCL11A gene and the donor repair template is incorporated into the BCL11A gene by homology directed repair (HDR) at the site of the double-strand break (DSB).

74. The method of any one of claims 71 to 73, wherein the cell is a hematopoietic cell.

75. The method of any one of claims 71 to 74, wherein the cell is a hematopoietic stem or progenitor cell.

76. The method of any one of claims 71 to 75, wherein the cell is a CD34⁺ cell.

77. The method of any one of claims 71 to 76, wherein the cell is a CD133⁺ cell.

78. The method of any one of claims 71 to 77, wherein the polynucleotide encoding the polypeptide is an mRNA.

79. The method of any one of claims 71 to 78, wherein a polynucleotide encoding a 5′-3′ exonuclease is introduced into the cell.

80. The method of any one of claims 71 to 79, wherein a polynucleotide encoding Trex2 or a biologically active fragment thereof is introduced into the cell.

81. The method of any one of claims 73 to 80, wherein the donor repair template comprises a 5′ homology arm homologous to a BCL11A gene sequence 5′ of the DSB and a 3′ homology arm homologous to a BCL11A gene sequence 3′ of the DSB.

82. The method of claim 81, wherein the lengths of the 5′ and 3′ homology arms are independently selected from about 100 bp to about 2500 bp.

83. The method of claim 81 or claim 82, wherein the lengths of the 5′ and 3′ homology arms are independently selected from about 600 bp to about 1500 bp.

84. The method of any one of claims 81 to 83, wherein the 5′-homology arm is about 1500 bp and the 3′ homology arm is about 1000 bp.

85. The method of any one of claims 81 to 84, wherein the 5′-homology arm is about 600 bp and the 3′ homology arm is about 600 bp.

86. The method of any one of claims 73 to 85, wherein a viral vector is used to introduce the donor repair template into the cell.

87. The method of claim 86, wherein the viral vector is a recombinant adeno-associated viral vector (rAAV) or a retrovirus.

88. The method of claim 87, wherein the rAAV has one or more ITRs from AAV2.

89. The method of claim 87 or claim 88, wherein the rAAV has a serotype selected from the group consisting of: AAV1, AAV2, AAV3, AAV4, AAV5, AAV6, AAV7, AAV8, AAV9, and AAV10.

90. The method of any one of claims 87 to 89, wherein the rAAV has an AAV2 or AAV6 serotype.

91. The method of claim 87, wherein the retrovirus is a lentivirus.

92. The method of claim 91, wherein the lentivirus is an integrase deficient lentivirus (IDLV).

93. A method of treating, preventing, or ameliorating at least one symptom of a hemoglobinopathy, or condition associated therewith, comprising administering to the subject an effective amount of the composition of claim 69 or claim 70.

94. The method of claim 93, wherein the subject has a β-globin genotype selected from the group consisting of: β^{E}/β⁰, β^{C}/β⁰, β⁰/β⁰, β^{E}/β^{E}, β^{C}/β⁺, β^{E}/β⁺, β⁰/β⁺, β⁺/β⁺, β^{C}/β^{C}, β^{E}/β^{S}, β⁰/β^{S}, β^{C}/β^{S}, β⁺/β^{S} or β^{S}/β^{S}.

95. The method of claim 93 or claim 94, wherein the amount of the composition is effective to decrease blood transfusions in the subject.

96. A method of treating, preventing, or ameliorating at least one symptom of a thalassemia, or condition associated therewith, comprising administering to the subject an effective amount of the composition of claim 69 or claim 70.

97. The method of claim 96, wherein the subject has an α-thalassemia or condition associated therewith.

98. The method of claim 96, wherein the subject has a β-thalassemia or condition associated therewith.

99. The method of claim 98, wherein the subject has a β-globin genotype selected from the group consisting of: β^{E}/β⁰, β^{C}/β⁰, β⁰/β⁰, β^{C}/β^{C}, β^{E}/β^{E}, β^{E}/β⁺, β^{C}/β^{E}, β^{C}/β⁺, β⁰/β⁺, or β⁺/β⁺.

100. A method of treating, preventing, or ameliorating at least one symptom of a sickle cell disease, or condition associated therewith, comprising administering to the subject an effective amount of the composition of claim 69 or claim 70.

101. The method of claim 100, wherein the subject has a β-globin genotype selected from the group consisting of: β^{E}/β^{S}, β⁰/β^{S}, β^{C}/β^{S}, β⁺/β^{S} or β^{S}/β^{S}.

102. A method of increasing the amount of γ-globin in a subject comprising administering to the subject an effective amount of the composition of claim 69 or claim 70.

103. A method of increasing the amount of fetal hemoglobin (HbF) in a subject comprising administering to the subject an effective amount of the composition of claim 69 or claim 70.

104. The method of claim 102 or claim 103, wherein the subject has a hemoglobinopathy.

105. The method of claim 104, wherein the subject has an α-thalassemia or condition associated therewith.

106. The method of claim 104, wherein the subject has a 3-thalassemia or condition associated therewith.

107. The method of claim 106, wherein the subject has a 3-globin genotype selected from the group consisting of: β^{E}/β⁰, β^{C}/β⁰, β⁰/β⁰, β^{C}/β^{C}, β^{E}/β^{E}, β^{E}/β⁺, β^{C}/β^{E}, β^{C}/β⁺, β⁰/β⁺, or β⁺/β⁺.

108. The method of claim 104, wherein the subject has a sickle cell disease, or condition associated therewith.

109. The method of claim 108, wherein the subject has a 3-globin genotype selected from the group consisting of: β^{E}/β^{S}, β⁰/β^{S}, β^{C}/β^{S}, β⁺/β^{S} or β^{S}/β^{S}.
